SEQUENCE LISTING (38 sequences)

SEQ ID NO: 1 - protein, 331 aa, Homo sapiens
  misc_feature: Human HER3 Protein amino acid sequence
MRANDALQVLGLLFSLARGSEVGNSQAVCPGTLNGLSVTGDAENQYQTLYKLYERCEVVMGNLEIVLTGHNADLSFLQWIREVTGYVLVAMNEFSTLPLPNLRVVRGTQVYDGKFAIFVMLNYNTNSSHALRQLRLTQLTEILSGGVYIEKNDKLCHMDTIDWRDIVRDRDAEIVVKDNGRSCPPCHEVCKGRCWGPGSEDCQTLTKTICAPQCNGHCFGPNPNQCCHDECAGGCSGPQDTDCFACRHFNDSGACVPRCPQPLVYNKLTFQLEPNPHTKYQYGGVCVASCPHNFVVDQTSCVRACPPDKMEVDKNGLKMCEPCGGLCPKAF

SEQ ID NO: 2 - protein, 1342 aa, Homo sapiens
  misc_feature: Human HER3 Protein Precursor amino acid sequence
MRANDALQVLGLLFSLARGSEVGNSQAVCPGTLNGLSVTGDAENQYQTLYKLYERCEVVMGNLEIVLTGHNADLSFLQWIREVTGYVLVAMNEFSTLPLPNLRVVRGTQVYDGKFAIFVMLNYNTNSSHALRQLRLTQLTEILSGGVYIEKNDKLCHMDTIDWRDIVRDRDAEIVVKDNGRSCPPCHEVCKGRCWGPGSEDCQTLTKTICAPQCNGHCFGPNPNQCCHDECAGGCSGPQDTDCFACRHFNDSGACVPRCPQPLVYNKLTFQLEPNPHTKYQYGGVCVASCPHNFVVDQTSCVRACPPDKMEVDKNGLKMCEPCGGLCPKACEGTGSGSRFQTVDSSNIDGFVNCTKILGNLDFLITGLNGDPWHKIPALDPEKLNVFRTVREITGYLNIQSWPPHMHNFSVFSNLTTIGGRSLYNRGFSLLIMKNLNVTSLGFRSLKEISAGRIYISANRQLCYHHSLNWTKVLRGPTEERLDIKHNRPRRDCVAEGKVCDPLCSSGGCWGPGPGQCLSCRNYSRGGVCVTHCNFLNGEPREFAHEAECFSCHPECQPMGGTATCNGSGSDTCAQCAHFRDGPHCVSSCPHGVLGAKGPIYKYPDVQNECRPCHENCTQGCKGPELQDCLGQTLVLIGKTHLTMALTVIAGLVVIFMMLGGTFLYWRGRRIQNKRAMRRYLERGESIEPLDPSEKANKVLARIFKETELRKLKVLGSGVFGTVHKGVWIPEGESIKIPVCIKVIEDKSGRQSFQAVTDHMLAIGSLDHAHIVRLLGLCPGSSLQLVTQYLPLGSLLDHVRQHRGALGPQLLLNWGVQIAKGMYYLEEHGMVHRNLAARNVLLKSPSQVQVADFGVADLLPPDDKQLLYSEAKTPIKWMALESIHFGKYTHQSDVWSYGVTVWELMTFGAEPYAGLRLAEVPDLLEKGERLAQPQICTIDVYMVMVKCWMIDENIRPTFKELANEFTRMARDPPRYLVIKRESGPGIAPGPEPHGLTNKKLEEVELEPELDLDLDLEAEEDNLATTTLGSALSLPVGTLNRPRGSQSLLSPSSGYMPMNQGNLGGSCQESAVSGSSERCPRPVSLHPMPRGCLASESSEGHVTGSEAELQEKVSMCRSRSRSRSPRPRGDSAYHSQRHSLLTPVTPLSPPGLEEEDVNGYVMPDTHLKGTPSSREGTLSSVGLSSVLGTEEEDEDEEYEYMNRRRRHSPPHPPRPSSLEELGYEYMDVGSDLSASLGSTQSCPLHPVPIMPTAGTTPDEDYEYMNRQRDGGGPGGDYAAMGACPASEQGYEEMRAFQGPGHQAPHVHYARLKTLRSLEATDSAFDNPDYWHSRLFPKANAQRT

SEQ ID NO: 3 - DNA, 4975 bp, Homo sapiens
  misc_feature: Human HER3 mRNA sequence
ctctcacacacacacacccctcccctgccatccctccccggactccggctccggctccgattgcaatttgcaacctccgctgccgtcgccgcagcagccaccaattcgccagcggttcaggtggctcttgcctcgatgtcctagcctaggggcccccgggccggacttggctgggctcccttcaccctctgcggagtcatgagggcgaacgacgctctgcaggtgctgggcttgcttttcagcctggcccggggctccgaggtgggcaactctcaggcagtgtgtcctgggactctgaatggcctgagtgtgaccggcgatgctgagaaccaataccagacactgtacaagctctacgagaggtgtgaggtggtgatggggaaccttgagattgtgctcacgggacacaatgccgacctctccttcctgcagtggattcgagaagtgacaggctatgtcctcgtggccatgaatgaattctctactctaccattgcccaacctccgcgtggtgcgagggacccaggtctacgatgggaagtttgccatcttcgtcatgttgaactataacaccaactccagccacgctctgcgccagctccgcttgactcagctcaccgagattctgtcagggggtgtttatattgagaagaacgataagctttgtcacatggacacaattgactggagggacatcgtgagggaccgagatgctgagatagtggtgaaggacaatggcagaagctgtcccccctgtcatgaggtttgcaaggggcgatgctggggtcctggatcagaagactgccagacattgaccaagaccatctgtgctcctcagtgtaatggtcactgctttgggcccaaccccaaccagtgctgccatgatgagtgtgccgggggctgctcaggccctcaggacacagactgctttgcctgccggcacttcaatgacagtggagcctgtgtacctcgctgtccacagcctcttgtctacaacaagctaactttccagctggaacccaatccccacaccaagtatcagtatggaggagtttgtgtagccagctgtccccataactttgtggtggatcaaacatcctgtgtcagggcctgtcctcctgacaagatggaagtagataaaaatgggctcaagatgtgtgagccttgtgggggactatgtcccaaagcctgtgagggaacaggctctgggagccgcttccagactgtggactcgagcaacattgatggatttgtgaactgcaccaagatcctgggcaacctggactttctgatcaccggcctcaatggagacccctggcacaagatccctgccctggacccagagaagctcaatgtcttccggacagtacgggagatcacaggttacctgaacatccagtcctggccgccccacatgcacaacttcagtgttttttccaatttgacaaccattggaggcagaagcctctacaaccggggcttctcattgttgatcatgaagaacttgaatgtcacatctctgggcttccgatccctgaaggaaattagtgctgggcgtatctatataagtgccaataggcagctctgctaccaccactctttgaactggaccaaggtgcttcgggggcctacggaagagcgactagacatcaagcataatcggccgcgcagagactgcgtggcagagggcaaagtgtgtgacccactgtgctcctctgggggatgctggggcccaggccctggtcagtgcttgtcctgtcgaaattatagccgaggaggtgtctgtgtgacccactgcaactttctgaatggggagcctcgagaatttgcccatgaggccgaatgcttctcctgccacccggaatgccaacccatggggggcactgccacatgcaatggctcgggctctgatacttgtgctcaatgtgcccattttcgagatgggccccactgtgtgagcagctgcccccatggagtcctaggtgccaagggcccaatctacaagtacccagatgttcagaatgaatgtcggccctgccatgagaactgcacccaggggtgtaaaggaccagagcttcaagactgtttaggacaaacactggtgctgatcggcaaaacccatctgacaatggctttgacagtgatagcaggattggtagtgattttcatgatgctgggcggcacttttctctactggcgtgggcgccggattcagaataaaagggctatgaggcgatacttggaacggggtgagagcatagagcctctggaccccagtgagaaggctaacaaagtcttggccagaatcttcaaagagacagagctaaggaagcttaaagtgcttggctcgggtgtctttggaactgtgcacaaaggagtgtggatccctgagggtgaatcaatcaagattccagtctgcattaaagtcattgaggacaagagtggacggcagagttttcaagctgtgacagatcatatgctggccattggcagcctggaccatgcccacattgtaaggctgctgggactatgcccagggtcatctctgcagcttgtcactcaatatttgcctctgggttctctgctggatcatgtgagacaacaccggggggcactggggccacagctgctgctcaactggggagtacaaattgccaagggaatgtactaccttgaggaacatggtatggtgcatagaaacctggctgcccgaaacgtgctactcaagtcacccagtcaggttcaggtggcagattttggtgtggctgacctgctgcctcctgatgataagcagctgctatacagtgaggccaagactccaattaagtggatggcccttgagagtatccactttgggaaatacacacaccagagtgatgtctggagctatggtgtgacagtttgggagttgatgaccttcggggcagagccctatgcagggctacgattggctgaagtaccagacctgctagagaagggggagcggttggcacagccccagatctgcacaattgatgtctacatggtgatggtcaagtgttggatgattgatgagaacattcgcccaacctttaaagaactagccaatgagttcaccaggatggcccgagacccaccacggtatctggtcataaagagagagagtgggcctggaatagcccctgggccagagccccatggtctgacaaacaagaagctagaggaagtagagctggagccagaactagacctagacctagacttggaagcagaggaggacaacctggcaaccaccacactgggctccgccctcagcctaccagttggaacacttaatcggccacgtgggagccagagccttttaagtccatcatctggatacatgcccatgaaccagggtaatcttggggggtcttgccaggagtctgcagtttctgggagcagtgaacggtgcccccgtccagtctctctacacccaatgccacggggatgcctggcatcagagtcatcagaggggcatgtaacaggctctgaggctgagctccaggagaaagtgtcaatgtgtagaagccggagcaggagccggagcccacggccacgcggagatagcgcctaccattcccagcgccacagtctgctgactcctgttaccccactctccccacccgggttagaggaagaggatgtcaacggttatgtcatgccagatacacacctcaaaggtactccctcctcccgggaaggcaccctttcttcagtgggtctcagttctgtcctgggtactgaagaagaagatgaagatgaggagtatgaatacatgaaccggaggagaaggcacagtccacctcatccccctaggccaagttcccttgaggagctgggttatgagtacatggatgtggggtcagacctcagtgcctctctgggcagcacacagagttgcccactccaccctgtacccatcatgcccactgcaggcacaactccagatgaagactatgaatatatgaatcggcaacgagatggaggtggtcctgggggtgattatgcagccatgggggcctgcccagcatctgagcaagggtatgaagagatgagagcttttcaggggcctggacatcaggccccccatgtccattatgcccgcctaaaaactctacgtagcttagaggctacagactctgcctttgataaccctgattactggcatagcaggcttttccccaaggctaatgcccagagaacgtaactcctgctccctgtggcactcagggagcatttaatggcagctagtgcctttagagggtaccgtcttctccctattccctctctctcccaggtcccagccccttttccccagtcccagacaattccattcaatctttggaggcttttaaacattttgacacaaaattcttatggtatgtagccagctgtgcactttcttctctttcccaaccccaggaaaggttttccttattttgtgtgctttcccagtcccattcctcagcttcttcacaggcactcctggagatatgaaggattactctccatatcccttcctctcaggctcttgactacttggaactaggctcttatgtgtgcctttgtttcccatcagactgtcaagaagaggaaagggaggaaacctagcagaggaaagtgtaattttggtttatgactcttaaccccctagaaagacagaagcttaaaatctgtgaagaaagaggttaggagtagatattgattactatcataattcagcacttaactatgagccaggcatcatactaaacttcacctacattatctcacttagtcctttatcatccttaaaacaattctgtgacatacatattatctcattttacacaaagggaagtcgggcatggtggctcatgcctgtaatctcagcactttgggaggctgaggcagaaggattacctgaggcaaggagtttgagaccagcttagccaacatagtaagacccccatctc

SEQ ID NO: 4 - DNA, 30237 bp, Homo sapiens
  misc_feature: Human HER3 DNA sequence
tttttttattgataattcttgggtgtttctcacagagggggatttggcagggtcatgggacaatagtggagggaaggtcagcagataaacaagtgaacaaaggtctctggttttcctaggcagaggaccctgcggccttccgcagtgtttgtgtccctgattacttgagattggggagtggtgatgactcttaatgagcatgctgccttcaagcatctgtttaacaaagcacatcttgcaccgcccttaatccatttaaccctgagtggacacagcacatgtttcagagagcacagggttgggggtaaggtcacagatcaacaggatcccaaggcagaagaatttttcttagtgcagaacaaaatgaaaagtctcccatgtctacttctttctacacagacacggcaaccatccgatttctcaatcttttccccacctttcccgcctttctattccacaaagccgccattgtcatcctggcccgttctcaatgagctgttgggtacacctcccagacggggtggtggccgggcagaggtgcccctcacctcccagacggggcggctggccgggcggggggctgacccccccacctccctcccggacggggcggctggccgggcggggggctgaccccccaacctccctccccgacggggcggctggccgggcggggggctgacccccccacctccctcctggacggggcggctgatcgggcgaggggctgacccccccatctccctcccggatggggtggctgccgggcggagactctcctcacttcccagatggggtggctgccgggcggagaggctcctcacttctcagacggggcagctgccggacggaggggctcctcacttctcagacggggtggttgccaggcagagggtctcctcacttctcagacggggcggccgagcagagacgctcttcacctcccagacggggtcgctgccgggcagaggcgctcctcatatcccagatggggcggcggggcagaggcgctccccacatctcagacaatgggcggccgggcagagacgctcctcacttcctagatgtgatggcggccgggaagaggcgctcctcacttcctagatgggatggcggcagggcggagacgctcctcactttccagactgggcagccaggcagaggggctcctcacatcccagacgatgggcggccaggcagagacactcctcacttcccagacggggtggcggccgggcagaggctgcaatctcagcactttgggaggccaaggcaggcggctgggaggtgtaggttgtagtgagccgagatcacgccactgcactccagcctgggcaccattgagcactgagtgaacgagactccgtctgcaatcccggcacctcgggaggccgaggctggcggatcactcgcggttaggggctggagaccggcccggccaacacagcgaaaccccgtctccaccaaaaccagtcaggcgtggcggcgcgtgcctgcaatcgcaggcactcggcaggctgaggcaggagaatcaggcagggaggttgcagtgagccgagatggcagcagtacagtccagcttcggctccgcatgagagggagaccgtggaaagagagggagaccgtggggagagggagagggggagggggagggggagggggagggaccaatcaacagtcttataagtagatacaacagtgtataaacaaggaaaccaaggaagatttttctccttcagaactcggaccctgaataccaggttgagctggagctgagtgagtaataaaatgaaaggccctttaatgtgggggagggtaggtaggagtggagacccttaagtagtatcagcactgttgtctgatgggagtgtgaatctgaacacatgaagctccagtctcagtagaacagtaagaaatcctaagtaaggccaggcatggttcacatctgaaatcctaacaatttgggtaaactgaggtggaaggattgcagaggccaggagttcaagaccagcttgggcaacatagccagacccccacccccacccccgcatctccatatcatacaaaaataataaagaaatcctaggtaaggccagatggtaaggccaggtgtggtggctcatgcctgtaatcccagcactttgggaggccgaggtgggtggattgcccaaggtcaggagttcaagaccagcttggtcaacacagtgaaaccccgtctctactaaaaatacaaaaattagctaggtgttgtggcaggcacctgtaatctcaactactcaggaggctgaggcaggagaatagcttgaacccaagaggcagaggttgcagtgagccaagatcgagccattgcactccagcctgggcaccaaaagcgaaactcaggagaatgccttgaacccaggaggcagaggttgcagtgagctgagatcacgccattgcactccagcctgggtgacagagcgagactccatctcaaaaacaaacaaacaaataaacaaagtagccagacattttggtgcccacctgtagactcagttactagggaggctgaagtgggagaatcacctgagcctgggaagttgaggctgctgtgagccatgattgcaccactgcactccagcctgggtgacagagggagatcctgtctcaaaaaaaggaaaaaaagccaggtgtggtggctcacacctgtaatcccagcactttgggaggctgaggtgggtggatcacctgaggtcaggaattagagactagtctggccatcacagtgaaaccccatctctactaaaaatacaaaaaattagccgggcttggtggcgcacgccttgtagccccagctacttggaagcctgaggcaggagaatcacttgaactcagtagtgagctgagatcaggccactgcactccagcctgggtgacagaaacagaacaagattccgtgtcaaaaaaaaaaaagcaacagaccagaaggccatgaggtcaaacaaaacaatgttttgtttttgttttgagatggcgtctcactctgtcgcccaagctggagtgcagtgatgcaatctcagctcactgcaacctccacctcccgggttcaagcgattctcctgcctcagtctcctaagtagctgggattacaggtgcccaccatcatgcccagctaatttttgtatttttagtagagacggtgtttcactatgttgactaggctggtctcgaactcctgacctcaagtgatctgcccgcctcggcctcccaaagtgctgggattacaggcatgagccaccgcgcccagccgaatgttttgttttttaagatggaagatatcccagcactttggcaggctgaagcaggtggatcattccagcttgggcaacaagagcgaaactctgtctcaaaaaaaaaaaaaaagaaaagaaaaaaaaagagagaaagaaaccctaggtaaaagtctgagccccacctcccaatcacctgatcacctcaaccactgtcaccaggtggatgaccttggagaggtcacacacttctagttctgtaaaatggggagttatattgcctaaatcatataatttttatgttaagtgacacattctctaaggcactaagtttgaggacatactttgtaaatgaaatgatatatggaaatgtttgtatatgttaatggttttgttgttgctattattattatgactactatacacatggtctggagaaagccaacctccccaaagcggagattctccagtagagaacaggccctctaggttgcatatcaatagggagcatgtttaaggaatgttagccggtagtctttgctaggtgtgaggggtgaaatttttctttatcaaggctcaactgttttcgaagtcttcaggcttgaagttctggagaaaacaactaggctctccgggcgagatcccgaataccagtttaagggatttgaaatgcaaggccgtctgggactccactgccacggatgggcaccaggcggcgccggtcggatccgtcccgggactagcagggctttgggcagcaacccgcagggagcccgaccgcctctggccaggtccgggcagctggtgggggaggttccagaggtccacgccattcgtggacgcagtctctagtgtcctctccgcgtcccacttcactgccccatcccctttcctgcgagagcctggacttggaaggcacctgggagggtgtaagcgccttggtgtgtgcccatctgggtccccagaagagcggcgggaactgcggccgcccggacggtgcggccagactccagtgtggaaggggaggcagctgttctcccaggcggccgtggggggcagcagaggggacggcgacaggtgcgggagcccctcccggggtagaagtggaaaggcgggctccggggtctgttcccaggctggaaaccacccccgccccccatccaaatccccgggagaggcccggccggcgccgggtctggaggaggaagcggccagagacagtgcaatttcacgcggtctctgtggctcgggttcctgggctgggtggatgaattatggggtttcgagtctgggagaaactgaggtggcctggacgtgaggcaaaaaacaccctccccctcaaaaacacacagagagaaatattcacattctgagagaaaatccaccaagtgaaccaaccggctaggggagttgagtgatttggttaatgggcgaggccaactttcagggggcagggctttggagagctttccactccctcattcattacccttccctggatctgggggctttcggaatctcgacctccccttggcctatctcctgcagaaaaattagggtgagccccatcctcgatctgctccgccaagttgcgggaccgcggggcgtggcacgctcggggcaggcggtccgaggctccgcaatccctactccagcctcgcgcgggagggggcgcggccgtgactcacccccttccctctgcgttcctccctccctctctctctctctctcacacacacacacccctcccctgccatccctccccggactccggctccggctccgattgcaatttgcaacctccgctgccgtcgccgcagcagccaccaattcgccagcggttcaggtggctcttgcctcgatgtcctagcctaggggcccccgggccggacttggctgggctcccttcaccctctgcggagtcatgagggcgaacgacgctctgcaggtgctgggcttgcttttcagcctggcccggggctccgaggtgggcaactctcaggcaggtaagtggcgcgagagcaccggcgggctcggcacctgggagccggaacccagtgcgcgcagcctcggagggtatgggcacggtctcaggcggcgcggggttgtgggtgctgcccccggtttgccaggaccacctgggagaggggcggtcaggctcgggttatcggcgtggtccggccgagggcggcattccgggaccctcacgccacccttctccagagcgtcgccgaccctctaattggtctccccagaagaggctgaggccgaaacagtagttcacacttctgaggggccctgcagggaggggagcagggaacttcattctgtaaacaggaggtgcttggaggtgggggccttggcgggaagggtctcggtttgctcgccaaccccctgccccccacccgcgccgatttcagctacccctagtttcgttgttttgccgacagggcggagctacagaaggttggagggggttgttgttctctggtgttggaaaaacaggagcggcacctcctcttccgtgagtgagcctgccctggggaggtctgagattaaccagagggccaagttcaggtgacatcaggcaggaggcccaacagaggctggcgccccctttcctcagtatagcagagcttaagcaacatctctttgtcaagacccaggtcaacacaactcatatttattgagcatctactatacacaaggccctgggccaggagctgtaagggcaaggatgtccagcctctggtcttttctctccccaacctgaggatcaagagggcacctctgctactttctaagcctcctgccttggggagtccttcctgggctcagcttgtgcctcccgcccccattttgcttattgtctgacactgtctctaggaccctagacagagccccggattgctcttctccagtcctcccccgactcccatgctatctgagcccacccctttggggtgtctctgggaccgtggacacctgaggactgaagttctgtggatctcctcccctcccctcagatctcagcttggggtttggcacagccagggccccttccccagtgtgggagtggaagaaaccacctgtgcttccctcacagttgctgggcctaaatttagatcctgggatttttagatgtgaacactcccagctggtggaggggggtggtctggggactggcgtgggagggagcagtggagtgactgtataactgtcccatccagactcctgcaatcttcacccagaaacccaggagtaccagagtctggccaccctccctggggaggcaggaggcaggcatggttgggtctctttaccctttatctgggtcctgcagcctctgggcatccggccctgtctcagtccttctatagcccctttgtcctggctgtgatgggggtgggggatgttggaggggaggcctctggttgaggaggggctggagattctggctctatcccacccctagtgctctctaccaaaggagggcctgtgacactgcccctccctatgctcccggttcctgggtacagcagggatttttatgattcccttcccctgccctgctccccaagctgcctagctcctccccagaggtgttgtttgtgctcccttcctgcccaggccctttgccccctgtttgtgtaatatggactttaccctcagggtagcagggaactgggctacctctaacactgtagccttccaagcacagacacaaagtctgcacaaacacttatgggcacgtgggatagatggggccacctgaaatacttcctgcaaggaaacccaactatagattcctgagccagcaggaccaatgtgtacgtgttcgtgtgtacattgtgtgtgtatgtgtgtgcccacactactacttccctgtgcagaaagcttggctcctcccttatctggggagaagtgttggcactataacttgggaaagggggtatcctgccaggaaggggattggggtggggctgttccagaaatgactaaccttcctagtctctttcatttcaacccaaggaccctggagttcccagctcctctggaactagctctctttgctgggactagccaaccttcatggggagtgataaggagccctccaaggtcaagaagtcagactaggggtgtatgttataggagggattggggcctcaccagtctccctccctccattcccactgttgcctcccactgagctaccaccgcctcagggaagggtggctggaacaggtggtatctaccccctactccccaccccacacatggtctttccctgaaccagaggaaagagactggggtagggcttcagagtccaggacttcccatagcccgttgtccaccacatttgcaaagaagagtgaactcccaaggctgacatgccatgtacctctagtctaggctctcccctagtgtgggtgaggattgccatggtgaaaggctttttcatgaacctcttctaacaatgaaattgtgtggaggctcaatatggggcatctgctactatctctctccaggttcctctgtatttgctgaaaaatactctagggctggaaagtgatgctgaggttgctagagtgtgttgggatgggggagagagtgagaaggaaaccctgagtttaggaaggcggggaggcaactagctccttatctttcagctttaaagacaaagctcccattgaccccccctcaccccagcactgccagagctcccccctctactgaggtcacttgtctgagcccaaggcttgagggtggaggggagtgctgctgaggacggggtgtctagggacagggtggggcagcccccctcctggatagaatcgcctcattgtgggctggactgtggccccaggcactgcccccaccctctgcccccatcccaccctcagtagacacaataggggctgtgtactagtcccaaagagatatttattccaggacctagagagaggcaggatgagggtagagaagtgagtgccctagttggagggggagaggagggtaatcaaagttgcggccttttcctaacttctcttttctagggagagaaacaaattccctgtcttccttctcagttaaccccttagtacccaaaagaagcacagaggggtcccaggttgaaaaaggaaatcttttcaccttcccattcatggaatggtaaggggattctgagaagagagaaagctctcaggccactacagcttctgcctatcgcttgtgggagggttggaggcaaatgccatctgatcctgtctaatgtaactggaagagggcaaccaagggggtgatctttggggatggcagatgggctgagaatttgtgtccagccctcagccactcttccctctgctttgaacagtgtgtcctgggactctgaatggcctgagtgtgaccggcgatgctgagaaccaataccagacactgtacaagctctacgagaggtgtgaggtggtgatggggaaccttgagattgtgctcacgggacacaatgccgacctctccttcctgcaggttagtgagcccaccctccttcctcaacctgctcctctttattctcccctagaaccctccttccttcttcagggctaccttctgctggagttcacccttcctaagactcaggagttcctaagattcaaaaccgtgtatttatggggacagtggctgtcatctgggacctatggtctcactgttgtagccagggatatatagggggcagggtcaggggcaggtggtgttctgtggatagtgcaaggtcagcagggactagtgcagagagaaacctgaggaccaagaggttacctggggagatgaggaaggggccctactggtatgaggcactttgaggagaaagctgcctgtcttcactcccagaagtgacacagcagtgtgacacagtctactccctactcccaaataggaattagcaagagttaaggccaggtgcagtggctcatgcctgtaatcccagcactttgggaggccaaggaaggcagatcacttgaggtcaggagttcaagaccagcctgggcaatgtggtgaaacgctgtctctacaaaaatacaaaaattagctgggtatggtggcatgcacctgtagtcccagctacttggagggctgaggtgggaggattgcttgagcccaggagtttgacgctgcagtgagcgagattgtgccactcgtaacagagcgagaccctgtctcaccaaaaaaaaaaaaaaaggccaagctcggatcacctgaggtcaggagttcgagaccagcctgaccaacatggaaaaaccacatctctactaaaaatacaaaattagccaggtgtagtggcacatgcctgtaatcccagctacttgggaggctgcggcaggagaattgcttgaacccgggaggtggaggttgtggtgagccaagatcgcactattgtactccagcctgggcaacaagagcgtaactccgtctcaaattttaaaaaaaagaaaaaaagaaaggaaagaaagaaggaagaattaaaacagttaaagagtctttaatgcctgaaggaggagaggagattgagattattttgccctgttgtctctctcatttacataatctgctctgtcacagtggattcgagaagtgacaggctatgtcctcgtggccatgaatgaattctctactctaccattgcccaacctccgcgtggtgcgagggacccaggtctacgatgggaagtttgccatcttcgtcatgttgaactataacaccaactccagccacgctctgcgccagctccgcttgactcagctcaccggtcagttcccgatggttccttctggcctcacccctcagccagcccaagactggtacctccttgatgatgacccaagactgctcactctaagtgcctcttccaaggtgcctgtcaccttggccgctgtctaaaggtccattgctccctaagcaatagagggcccccagtagggggagctaggggcatctgctccagggaaaggaaccctgtgtccttgtggggctggagtcagagctggatctgttaaccgtttttctaatttcaaagtacagtgtaccggaggccaggcctgatggcttacacctgtaatcccagcattttgggaggccaaggagggcagatcacttgagatcaggagtttgagaccagcctggccaacatggcgaaaccctgtctctactaaaaatacaaaaaaataaaataaaataaaaaattagctggctatagtggtgcgcacctgtaatcccagctgttcatgaggctgaggcaggagactcgcttgaacctgggaggtggaggttgcagtgagctgagattgcaccactgcactctagcgtaagtgacagtgagactccgtctcaaaaaaaaaaaaaaaaaaaaaaaaaagcctgggcgcggtggctcacgcctgtaatcccagcactttgggaggccaaggcaggtggatcacaaggtcaggagatcgagaccatcctggctaacacggtgaaaccccgtctctactaaaaatacaaaaaattagccaggtgtggtggcgggcgcctgtagtccgagctactcgggaggctgaggcaggagaatggcgtgaacccgggaggcggagcttgcagtgagccaagattgcaccactgcactccagcctgggcgacagagcgagactccaaaaaaaaaaaaaaaaaaaaaaaccaaagtacagtatacctggatgtccctccttccctaggaattctacctttactctcctaaaccaaacccctatgagctggaggaatataggggttaaaaaccacctgtccatcttctgcttctccatgtcccagtcagttggaaaacatatgggcagggcttgggggagggaatgttgagtcagaaatctctccctctctcccttcccctcccccactagctaaaccggatctggacaggtgactgaggaggcaggagtttcttttggcctgactcctcatcttataaagggagtcttctctgcagcttagatttaattggacctatctgtctgcctcattctcccactcctgagtctcaggtgtccttttggatgggtggagaggtaaggaagaggcgttccgctgcggcccttaaccctgtcacttctttccctacctcagagattctgtcagggggtgtttatattgagaagaacgataagctttgtcacatggacacaattgactggagggacatcgtgagggaccgagatgctgagatagtggtgaaggacaatggcagaagctgtaagtggccgtgatcaagattgctccccagtcccaccaaaccagagtgactcccttctttccatcatccttacattcctgatctgaacccgcctccccagtgaacaaacacctcaggtccctgactcagcagcccaccagggcagaccattccagtctcctggaatctaaaccacagaggaggtgtttcaagaaaaggagcaggccgagcatggtggctcatgcctataatcctggcactttgggaagccaaggtgggaggatcgcttgagcccaggagtccaagaccagcctgagtaacatagcaaaaaatctacaaaaaattaaaaaaatcagacaggcgtagtggctcgcacctgtaatcccggctactcaggaggctgaggcagaggattgcttgagcccaggaggctgcagctgcagtgagctgtgattgtgccattgtactctagcctgggcaacagagtgagaccctgtctcaaaaaaaaaaaaaaaaaatccctgagtactaagcagggaagccagatctttagactccacatctgttactcgttccactagaatatactcccatttccttggagcccaccttcccctgaccattcacatgcatatattcctgcatatattcagtttgcccaggaggaactaagttcctgggttgggactaggactaaggttggcatttgccccagtccctccccttcagctgcccagtgggtggtgtggaggcgtggccgcgccccttgttgacaggtccacttgagcccagccctgctctccaagggcagggagggacacagccctggctttttgcttcccgggattgaggtgcctgtgtactgacatcataccccgttgattaaaacaagcctttcttagccctgatggccccttgtgttgccttccttcccaaccaggtcccccctgtcatgaggtttgcaaggggcgatgctggggtcctggatcagaagactgccagacatgtgggtttgaaattccctccaaaaacttcactcatacgctttcatatcccttcctccccaagcctgggtcaacactgtgggggaggcatgagcagtggcctcagaattcagtcctaggagccctaacagccatgctttctctccttccatagtgaccaagaccatctgtgctcctcagtgtaatggtcactgctttgggcccaaccccaaccagtgctgccatgatgagtgtgccgggggctgctcaggccctcaggacacagactgctttgtatgtaccctttccattgcctgggttctgaaattgggatgtggcctttgaggaggaggtaggggtacacacgtaacataaatctgatgagcctccttttttcccaggcctgccggcacttcaatgacagtggagcctgtgtacctcgctgtccacagcctcttgtctacaacaagctaactttccagctggaacccaatccccacaccaagtatcagtatggaggagtttgtgtagccagctgtccccgtaagtgtctgaggggaaggaacaatgatcaacaatagtagatccaagattttagacaaaattgtggaagggaaaaagaatccagttggtgataaatagggagattggtgaatggttatgatcatctaaccactccagtgagtgacccttacgtccagtcctcccatgacttcagctatcacccttacttctgctccttgtagcaacaaatagtgaagagacttttgaatctatagggcagcacttaagggatctagggtggcagatggggacaaatccagtgcagagctggagggagcctaggcccagagcaagggttccattggtagctggtgatgttcctccctcatctctaatggtgtcctcctcctcttccctagataactttgtggtggatcaaacatcctgtgtcagggcctgtcctcctgacaagatggaagtagataaaaatgggctcaagatgtgtgagccttgtgggggactatgtcccaaaggtgggtaggagatggtaagaagttgtaaagagacagcctttcctctgagcctgcgcagaccacccccactgaacctctcttacatttgcagcctgtgagggaacaggctctgggagccgcttccagactgtggactcgagcaacattgatggatttgtgaactgcaccaagatcctgggcaacctggactttctgatcaccggcctcaatgggttagagatcctgccttccctccttagaccccagcccacgcacccctcacagttcatttcattggccaaaactttcctatgtggagctgactaggaatcaaagtcataaaattctagcctgttacaaaggacctgaaagaatgcttaacacatcctccatccaggccttcggtcccctcaggaacatctttgagcaattcaatatcgccctgccaaggaacaagggacaggaacaacatatcctccttcttaaagttttcttttttattcttttttcttttttgagatagggtcttgctctgtcacctaggctggagtgcagtggcgtgatctcgactcactgtagcctcgacctcctgggctcaagtgatcccaagtagctgggactataggcacacaccatcatacttgactaatttttttgtatttttttgtagagacagggtcttgctatgttgcccaggctgatctcgaactcctgtgctcaagcaatcctcccatcttggcctcccaaagtgctagggatcacagcacccaacctccttcttaaagttttgtaaaagttcttccttagatttggataaaaatctgtctccaggctgggcccggtggctcatgcctataatcccagcactttgggaggccgaggtgggcggattacgaggtcagatcgagaccatcctggctaacatggtgaaatgccatctctactaaaaacacaaaaattagctgggtgtggtggtgcacatgcctgtaatcccagctactcaggaggctgaggcacgagaatcacttgaacccaggaggcggaaattgcagtgaaccgagattacaccaccgcactccagcctggcgacagagcgagactctttctcaaaaaaaaaaaagaaaagaaaagaaaattctgtctccccatgacttttagctgttttcactcattctgctccttggagcaaaaagaacaaagggactttctagtctataggacagcatttaaaatgtgtgtgtgtgtgtaaaaaaaacccactatgaccacctgtttttttttttcctttaattttttattttgacataattttagatctacactaaagttgcaagaatggtataaaattccccatatactttttttttttttttaagacaaagtctcactctgttccccaggctagagtgcagtggtgcaatcttggctcactgcaacctccgcctcctctgcctcccgggttcaagcaattctcctgcctcagtctcctgagtagctggaattataggtgtgtgccatcatgcccggctaatttttgtatttttagtagagacagggtttcaccatgtaggccaggctggtctcgaactcctgacctcaagtgatccacccgccccagcttcccaaagtgttgggattacaagtgtgagccaccgcgtctggccccccatacactcttttacccagatcctccaaatgttaacataccacatatggccgggcacagtggctcatgcctataatcccagcactttgggaggctgaggcaggtggatcactaggtgtggatcacgaggtcaagagattgagaccatcctggccaacatggtgaaacctcatctctactaaaaatacaaaaattagctgggcgtggtggtgtgcgcctgtagtcccagttactcaggaggctgaggcaggagaatagcttgaacctggcaggcagaggttgcagtgagccgagatcgcggcactgcactccagcctggtgaaagagcgagactctgtcccccgccaaaaaaaataccacatacgctttatcacttctctctctctctgtctctctctacacacacacacacacacacaaaacacatgctattgtttttctggaccacgtgagggtaaattttcacacatggttctttcttacccctgttatatttcagcgtatattccttaaaaataatattttcttacataaccacagcatagttgtttgaatcggaaaattaacattaacacaaaatattatctaagctacagaccttattcagatttcactaattgtcctcctaaggtttgggatcatacattacattcagttatcgtggcacttcaatctcctttataacagctcctcaggttttgtttatctttcatgatattcttgatgagtatagattaggtaatgggcagcatgttcttcagtttggattagtttgatgtgtcctcatgattagattcaagtttttgtagtttttttttgagacaaggtctggctctattgcccaggctggaatacagtggcatgatctcagctcactgcaacctctgcctcccgtgctcaagcgagcacctcagccccctgagtagctgggattacaggtgcatgccaccatgcttggctaattttatatatatatatatatatatatatatataaataatatatataaatataaatatatatatataaataatatatataaatatatataaacataaatattatatactatttatatatttatatatgtgtgtgtatatatatatatatttttttttttttttgagacggagtctcgctcttgttgcccaggctggagtgcagtggcgtgatctcagctcacgcaacctccacctctcaggttcaagccattctctatagagacagggttgcaccatgttgtccaggatggtctcgaactcatgagctcaagtgatcctcctgtctcagcctcccaaagtgctgggattataggcatgcgccgctgccaggctggagtttgataagaacaccacagaggctgtgagctcagggcatcctattgaggatgtacgtgatgttgatttgtcccagcactcacaatgatgtctctggtcacttagttaaggtgatatctgtcaggtttttctactgtaaagttactatttttccattcacaattaatgaatgtcttgggataattgcctgaatcaattattgttatgatagttgccaaatgataattttctaattccattattccttctgcatttgtttgttggcattctactgttaggaagagtctttccagctgagcacagtggcttatgcctgtaatctcagccctttgggagccagtgggaaaattgcttgggcccaggagttcaaggttacagtgagctatgatggcactactgctctccagccagtgcactcactctgcacaacagagtgagaccctgtctcttaaaaaaaaaaaaaaaaaaaggccaggtgcagtggctcatgcctgtaattccagcactttgggaggccgaggcgggcggatcacaaggtcaggagttcaagaccagcctggccagcatggtgaaaccctgtctctactaaaaatacaaaaaattagccaggcatggtggtgtgctcctgtattcccagctacttaggaagctgaggcaggagaatcacttgaacccaggaggtggaggttgcagtgagctgagattgctgccactgtactccagcctgggcgatagagcaagactctgtctcaaaaaaaaaaaaaaaaaaaaaaaaaaaaaaaggtcttcctttcctatttactcttatggatacttattttattctagtcaatggttataatcctttacaatcattatttattttagccagcccctccaagttggctcctgtgttctttgggctgtacccttaattctttgagtcttgtcttgtttgcacaagatgctctgggcttatattatatttcccccatcccagccatttctccaaggaatgtttccttttagtggagaatgatatttagaaaccaaatgctgaggctgggtgtgctcattgccattgagttatacctttaccttattgactggtttctactgttctattcagagacccctggcacaagatccctgccctggacccagagaagctcaatgtcttccggacagtacgggagatcacaggtgagtggcagagagtttgccctttctagaagaataggtgaaccactggcataaattgcggtataactacttgagaaaatcacgtcccaagttataggggaggagccaggagaacccaagaaagaagaaggctccctgcccatatgcctctctccaacccctcaggttacctgaacatccagtcctggccgccccacatgcacaacttcagtgttttttccaatttgacaaccattggaggcagaagcctctacaagtgagtaaagggtatggaggaaatggcatcttcaggcaatgaagcctgtgtcataggcattctttagtaaaatacaaggcactgtctcatacagcagtgcctcaaaaccaaagggtttcagagtttcacgaggaaaaggcaaaaggagggggattccctctcagtggatctgactagcactgagcaaatttcttgactaacatgaatcctttgaatagttaatgttcccttagtagtctctcctctcatcctgtctccttattctcagccggggcttctcattgttgatcatgaagaacttgaatgtcacatctctgggcttccgatccctgaaggaaattagtgctgggcgtatctatataagtgccaataggcagctctgctaccaccactctttgaactggaccaaggtgcttcgggggcctacggaagagcgactagacatcaagcataatcggccgcgcagagactgcggtgagggaaagggtctgctaggtggtgagaatagggagtcagggaggagagggctgaaaggactattctgccctagacgtgggagtagggttgagggatggaaccaaggagaagggggctgttaggctggaagcagtaacgaggaagaataatgaagagagggcttgctgggagtcctcagactcctctcctaacccaccccttcctttccagtggcagagggcaaagtgtgtgacccactgtgctcctctgggggatgctggggcccaggccctggtcagtgcttgtcctgtcgaaattatagccgaggaggtgtctgtgtgacccactgcaactttctgaatgggtacagtaaggggagccagtcaaggatgggtgggggtggggccctgcaatggaactgttcaggtggcatacaataaaagtctttagacagctttctgcatgtgccttggtgggattgaggtaggagacctgtggttgtgagatcggagcatgaaggtcaggacttggaagtgacccccccctccctttattccccactacagggagcctcgagaatttgcccatgaggccgaatgcttctcctgccacccggaatgccaacccatggagggcactgccacatgcaatggctcggtatactagtagcaccaggatctccaagggagacagagaaggggcaatacttggagcatctggggaatgatatggctaaggatagcacagagaggccagataatgctagggcctgcagatagaagatcctgaatgtctgggttggtctttgctgggaggtatggaattgaccttgggatctgattcttcctgaccttctctcttccactcagggctctgatacttgtgctcaatgtgcccattttcgagatgggccccactgtgtgagcagctgcccccatggagtcctaggtgccaagggcccaatctacaagtacccagatgttcagaatgaatgtcggccctgccatgagaactgcacccaggggtcagtgatgggataataaggagagggggtcaggtggaagggtaggagcacagaactagagtgagggaagcagaaagaagagagaggctgtgattcaagaatcactcccagctggccgggcgcagtggctcacacctgtaatcccagcactgtgggaggccgaggtgggtggatcacctgaggtcgggagttcgagaccagcctgaccaatatggagaaaccctgtctctaccaaaaatttaaaattagcccggcgtggtggcgcatgcctgtaatcccagctacgcgggaggctgaggcaggagaatttcttgaacccaggaggcagaggttgcggtgagctgagattgcatcattgtactccagcctgggcaacaagagtgaaactctgtctcaaaaaaaaaaaaagaatcactcccagctgtgtagcgaaggattggagaaagggaaaatcagtaacagcacaaaattacaccacagttttgggaacctggaataacctcagttcaagggagtttcacagaagaggggcttggggagagctagtgagctggaggtggaggccatgtcttgggatcagctctgggctccaggatgggatgccacggtaagttctgaaacaagcttttatatgttaggctgttgaaattgagcctctgctgtccaagctctcatttaaggtggtgactttcttccctaggtgtaaaggaccagagcttcaagactgtttaggacaaacactggtgctgatcgggtatgatggggttggagattctggaaactggggatatttgggagttgggagagaggtggttacctggagagaagagggaggctgtcttcattctggccttttatgtatgcagtccactatcactggacacttgggactcaagaatgcaggcttctggacttcccttcctaaaattaactttcagtagtctaagactggtccagatttaggttggtcccttcagtgcttaaggatatatatgtgaatgttaatttcttgccccaggtcagcatcataccttcaacacaagtatagttgacatttgtaaggaagatgcaaacccaggataatgttgggtttctatatatcccatagcaaaacccatctgacaatggctttgacagtgatagcaggattggtagtgattttcatgatgctgggcggcacttttctctactggcgtgggcgccggattcagaataaaagggctatgaggcgatacttggaacggggtgaggtgagtacttagcttacttttgttttttcttttctttttttgcatgtcctggaagtctctttatagcttaattttgagtggtaccctgtgcacccaggggtcagtgatgggataaatgtcactcccctcctctttccccagagatttgatccctttcttcaaggaagtagtgtggtcccctagaagaacactggtcagagaaatgggaggcatgcattctagtcctgattttgccattaatttgccacatgactttgaagaagttacttatcttctctgtgcctcggtttatgcatctatacagaggaaataacatttgtccttccaggatggctgtaagggtaaagggggatgatgtatgtgaaagtgctttggaaagcacagagcactgtataaaaggtactcaaggtggtaatagtactaccaactctccctagctgtccccttccccactttgtgctcctccatcaaagggaaaacccaacccctttgattcctgatctcatgagcacaaataacttcctcagttctcagggtctgtacctcaatatgcctataatccattccaggactaacggtgcttcctcttcctgccctttcagctgtgctgcttttggcattcacctatgaggagcgggttggagtgggacatgggaatggcctttcctgagtaactccttcccatttgctcctcagagcatagagcctctggaccccagtgagaaggctaacaaagtcttggccagaatcttcaaagagacagagctaaggaagcttaaagtgcttggctcgggtgtctttggaactgtgcacaaagtgagtgacccataggaattctggagaggtggggaaggcatctagggcaaaggggtgaaagatttttgcataggattgacctagggagaatgaccttatgccaactcctgccccaaacttcccagggagtgtggatccctgagggtgaatcaatcaagattccagtctgcattaaagtcattgaggacaagagtggacggcagagttttcaagctgtgacagatgtaagtgaaggaaattctgtatgccgctaggagagaggacaatattagatacaatcatgtagaagcagggtcctgtgcttctcagcagctactatgttagccagaatgttgggggtgggggggcctgggctggctgtgcacatgctgagtgtatgtgaacctgttggtttcctagataataccttttgtgtctcttagcatatgctggccattggcagcctggaccatgcccacattgtaaggctgctgggactatgcccagggtcatctctgcagcttgtcactcaatatttgcctctgggttctctgctggatcatgtgagacaacaccggggggcactggggccacagctgctgctcaactggggagtacaaattgccaaggtgagagaagcctggaggaattctgtgataagaactgcttgtctgggggccagccaggaaaaagtgagaaggttgaagttctgagaggtgaggtccccaacccccgggctgcagactggtaccagtccatggcctgttaggaaccaggccacagagcatgtgagcggcaggcaagcgagtgaagcttcatctgtatttacagtcagtccccatcacttgcattaccgcccgagttccgcctcctgtcagatcaggggcagcattagattctcttaggagcttgacttctattgtgaactgtgcatgtgaaggatctaggttgtgcactccttatgagaatctaactaatgcctgatgatctgaggtggaaaaatttcatcccaaaaccaaccctccccttcccctggaaaaactgtcttccacaaaaccagtccctggtgccaaaaaaggttggggaccactgctgagaggtaccttcaagatttgggggaattccagatctcagtgactgattcccccaaccttaagaatactttcttcccctatacctacagggaatgtactaccttgaggaacatggtatggtgcatagaaacctggctgcccgaaacgtgctactcaagtcacccagtcaggttcaggtggcagattttggtgtggctgacctgctgcctcctgatgataagcagctgctatacagtgaggccaaggtgaggagacacaaagggtaaggaggcgggggtggagtgaagcatggggatagggagcagccagtggtctcttccagaggcaagcagatgcttcatggtaagttcaaggagagaaggctgcagatgccagatattttagttcagagggcaacaaataaaataatgatcaagaacttgggactggccgggcgcggtggctcacgcctgtaatcccaacacttcgggaggccaaggcgggtggatcacaaggtcaggagatcaagaccatcctggctagcacggtgaaaccccgtctctactaaatatacaaaaaaaaaaaaaattagccaggcgtggcggcatgcatctgtactcccagctactcgggaggctgaggcaggagaatggcgtgaacccaggaggcggagcttgcagtgggccgagatcgcaccactgcactccagtctgggcgacagagcgagactccgtctcaaaaaaaaaaaaaaaagaatttgggacttggaaatcctaagaaaatttgtggaaataaacttgtgatacctctatctttaatccgcagactccaattaagtggatggcccttgagagtatccactttgggaaatacacacaccagagtgatgtctggagctatggtcagtgcatctggatgccctctctaccatcactggccccagtttcaaatttaccttttgagaccccctcttagaatctctaagcacttcagatttttgtgttagatcaggttctgccttcccttcacttcatgcccatgtctactattttgccagtgactagtccatgtcttcctgcaacaggtgtgacagtttgggagttgatgaccttcggggcagagccctatgcagggctacgattggctgaagtaccagacctgctagagaagggggagcggttggcacagccccagatctgcacaattgatgtctacatggtgatggtcaagtgtgagttacctgctgagcccaaccattttctctttttttctttttttttcttttttttttttttttgagacagagtctcacaattgtcacccaggctggagtgcaatggtgcaatcaatcttggctcactacaacctccgcctctcgggttcaagagattctcctgcttcagcctccggagtagctgggattacaggcgcccgccacacctggataactgttacacttttagtagagatggggtttcaccatgttggccaggctggtctcaaactcctgacctcaggtgatccgcctgcctcagcttcccaaagtgctgggattacaggtgtgagccatcatgctcggcctgactgcagccattttctgacttccctctgtactcctcttatggctctattccttttttttttatggagtctcgctctgttgcccatactggagtgcagtagcgtgaccttggctcaccgtgacctccacgttccaggtttaagttcttctgtctcagcctcccagatagctgggactttaggcgtgcaccaccacgcccagctaatttttttttgtctttttagtagagatggggtttcactatgttggccaggctggtctcaaagtcccgacctcaggtgatccacccgccttggcctcccaaagtgctgggattataggtgtgagccaccgcgcccggccatggaatgtattctcttttatgtctctacctcctacatcttatctccaggttggatgattgatgagaacattcgcccaacctttaaagaactagccaatgagttcaccaggatggcccgagacccaccacggtatctggtcataaaggtgagtagggagtaggaggtgctaaggaaatttagaaaaaggaggagttggctggaaccaggattccccctaacaatcacctatcgatatagagagagagtgggcctggaatagcccctgggccagagccccatggtctgacaaacaagaagctagaggaagtagagctggagccagaactagacctagacctagacttggaagcagaggaggacaacctggcaaccaccacactgggctccgccctcagcctaccagttggaacacttaatcggccacgtggggtaagacaacttctaattacccaacactttgcaccctgagccctcacaaaccctacagatacccagattaactactcaaaggcccccatggtgaatgtagatttctcccttcatcttaaccttttccttattttttcatcctagagccagagccttttaagtccatcatctggatacatgcccatgaaccagggtaatcttggggagtcttgccaggtaagttctgttgctgagaggctgggttttaggatcagattgatacgagtagtatggaagacattagaaacctctgaggtttaatcagtgtcctgcaaaaaagaaggcagtgagggccgggcgagttggctcacacctgtaatcccagcactttgggaggccagagagagtggatcacctgaggttaggagtttgagaccagcctggccaacatggtgaaaccccgtctctacccaaaatacaaaaattagctgggtgtggtggtgcacacctgtaatcacagctactcaggaggctgagacaggagaatcgcttgaacccgggaggcagaggttgcagtgagctgagattgtaccactgcactccagcctgggtgacagagcaagaccctgtctcttaaaaaaaaaaaaaaaaggccaggtgcggtggctcacgcctgtaatcctagcactttgggaggccgaggtgggcggatcatgaggtcaggagttcgagaccagcctgaccaacatggcaaaaccctgtctgtactaaaaatacaaaaactagctgcacatgatggcaggtgcctgtaatcccagctactcgggaggctgaggcaggagaatcacttgaacagggaagcagaggctgcagtgagccaagataatgccactgcactccagcctgggcgacaagaacaagactccacctcaaaaaaaaaaaaaaaaaaaaaaaaaggcagtgaacaacccaatatccttctaaacaaatctctcttctttcctcatcatgtaaatttccttgcattattttctgtttattttcttccttaggagtctgcagtttctgggagcagtgaacggtgcccccgtccagtctctctacacccaatgccacggggatgcctggcatcagagtcatcagaggggcatgtaacaggctctgaggctgagctccaggagaaagtgtcaatgtgtaggagccggagcaggagccggagcccacggccacgcggagatagcgcctaccattcccagcgccacagtctgctgactcctgttaccccactctccccacccgggttagaggaagaggatgtcaacggttatgtcatgccagatacacacctcaaaggtgcctgactcttcctagggctttcctcaatttttcctcgaattctttccccgggctcctcttttttcttctctgatcatatgcctctctgtcctattaattttttcaaactttcccctaccctcatgaagttcttcacatacctagcctttcttctcaacccccaggtactccctcctcccgggaaggcaccctttcttcagtgggtctcagttctgtcctgggtactgaagaagaagatgaagatgaggagtatgaatacatgaaccggaggagaaggcacagtccacctcatccccctaggccaagttcccttgaggagctgggttatgagtacatggatgtggggtcagacctcagtgcctctctgggcagcacacagagttgcccactccaccctgtacccatcatgcccactgcaggcacaactccagatgaagactatgaatatatgaatcggcaacgagatggaggtggtcctgggggtgattatgcagccatgggggcctgcccagcatctgagcaagggtatgaagagatgagagcttttcaggggcctggacatcaggccccccatgtccattatgcccgcctaaaaactctacgtagcttagaggctacagactctgcctttgataaccctgattactggcatagcaggcttttccccaaggctaatgcccagagaacgtaactcctgctccctgtggcactcagggagcatttaatggcagctagtgcctttagagggtaccgtcttctccctattccctctctctcccaggtcccagccccttttccccagtcccagacaattccattcaatctttggaggcttttaaacattttgacacaaaattcttatggtatgtagccagctgtgcactttcttctctttcccaaccccaggaaaggttttccttattttgtgtgctttcccagtcccattcctcagcttcttcacaggcactcctggagatatgaaggattactctccatatcccttcctctcaggctcttgactacttggaactaggctcttatgtgtgcctttgtttcccatcagactgtcaagaagaggaaagggaggaaacctagcagaggaaagtgtaattttggtttatgactcttaaccccctagaaagacagaagcttaaaatctgtgaagaaagaggttaggagtagatattgattactatcataattcagcacttaactatgagccaggcatcatactaaacttcacctacattatctcacttagtcctttatcatccttaaaacaattctgtgacatacatattatctcattttacacaaagggaagtcgggcatggtggctcatgcctgtaatctcagcactttgggaggctgaggcagaaggattacctgaggcaaggagtttgagaccagcttagccaacatagtaagacccccatctctttaaaaaaaaaaaaaaaaaaaaaaaaaaaactttagaactgggtgcagtggctcatgcctgtaatcccagccagcactttgggaggctgagatgggaagatcacttgagcccagaattagagataagcctatggaaacatagcaagacactgtctctacaggggaaaaaaaaaaaagaaactgagccttaaagagatgaaataaattaagcagtagatccaggatgcaaaatcctcccaattcctgtgcatgtgctcttattgtaaggtgccaagaaaaactgatttaagttacagcccttgtttaaggggcactgtttcttgtttttgcactgaatcaagtctaaccccaacagccacatcctcctatacctagacatctcatctcaggaagtggtggtgggggtagtcagaaggaaaaataactggacatctttgtgtaaaccataatccacatgtgccgtaaatgatcttcactccttatccgagggcaaattcacaaggatccccaagatccacttttagaagccattctcatccagcagtgagaagcttccaggtaggacagaaaaaagatccagcttcagctgcacacctctgtccccttggatggggaactaagggaaaacgtctgttgtatcactgaagttttttgttttgtttttatacgtgtctgaataaaaatgccaaagttttttttcagcttcctgtctgtcaaatgaagacatttcgtatgttagataagagatctgctcctcagcagtggatactcacctttctgtgttctgacagtgctactctgtcccatgcagctttctctagtcctactattacttctatttctttagaacaaccatagcgcatagtccttttcattaagggttttagtaggaatctacaaggcaaccaattgggaataacaaaaagaacctacgtgctttaggacttataaaaagccctataagccctccttcagaggccaaacactgaaacctccagatgcttctgaattcattatcttagaaaagtcatcaaatctttttattttttcacggtaagaactctcaacaaacatgtctttctgaacacttcccttaggtgctccatccaggtgcctgttattggaacaataaagtcatgttacttcattaggagtccggcctctagattgcgaggcctttaaatggatgatccctccggtgtctggctgcccagttagcccccgttaccagcacccttggtcttcttccacctgtctgcccctccctgttctcccagcttcggaggacgactggaccggctgggcgggtttcgccagccgacccagggatccgaagaagggcgcacccagcctccccgacctaggtgtagacactgcccacccgctgcggctccactctactccacccctgcccgctcgactttaaacctatttccccgccgtagctccgcccctctcccctcagcccgcccctctctgttactggctctcgctcagcgttctcggtggaagtggtttttccgggagagaccacgcttcccctcaagctccccaacggctccgccttcccgccggagcctgacccttcccagagtgcccggcgattccggcgtgcgaggcccttggagggcaaggccccagggcctggcttaggagcgcgagaggcaggctgggaattgtagttcgaaggccctcgagagcggctagagtctggcggccgagaggactagttgtcccagcgtgccctgcgcctcagcccgcgcgctcgcagcttctcgctctcgcctgcctgcccgctcccttgcttgctcgcgctttcgctcgccctctcctcgaggatcgaggggactctgaccacagcctgtggctgggaagggagacagaggcggcggcggctcaggggaaacgaggctgcagtggtggtagtaggaagatgtcgggcgaggacgagcaacaggagcaaactatcgctgaggacctggtcgtgaccaagtataagatggggggcgacatcgccaacagtgagtgcggcctcgggggtcggggaatcaaggctgatagggaaaggtaacaggctggcccggaaggggctggagcggaggggtcatgcggactgagctactgaggggcccgcaccggtccgctgggcacggcgtggtgggaagacccggtgtcgcgcctgggacctgagcgggcaggcccaggctgaagtctatggaggtgcgggtcggcgaccaggatgagcgcagagaggggaccctggcaggctccgacccgaggccgtttgttaggaggcaagacgtgttttctcttgttcctatccttcattcccgattattgcttcctctgattctggcagcggcgaggccacctcgaaaaggaagcgcccagctattggcgacagaagtgcccctctgtttttgtagcgtctccggggcgtcaggagccaagccggcacgtgttgctgggtcccattgcggatggctggcccccttcttactccgctagtgtccctgacaccagcttccccaccaccagct

SEQ ID NO: 5 - protein, 11 aa, Artificial Sequence
  Synthetic peptide derived from HER3 protein sequence
NLRVVRGTQVY

SEQ ID NO: 6 - protein, 15 aa, Artificial Sequence
  Synthetic peptide derived from HER3 protein sequence
DKLCHMDTIDWRDIV

SEQ ID NO: 7 - protein, 7 aa, Artificial Sequence
  Synthetic peptide derived from HER3 protein sequence
PCHEVCK

SEQ ID NO: 8 - protein, 15 aa, Artificial Sequence
  Synthetic peptide derived from HER3 protein sequence
ICAPQCNGHCFGPNP

SEQ ID NO: 9 - protein, 7 aa, Artificial Sequence
  Synthetic peptide derived from HER3 protein sequence
NGDPWHK

SEQ ID NO: 10 - protein, 11 aa, Artificial Sequence
  Synthetic peptide derived from HER3 protein sequence
DPLCSSGGCWG

SEQ ID NO: 11 - protein, 11 aa, Artificial Sequence
  Synthetic peptide derived from HER3 protein sequence
CPHGVLGAKGP

SEQ ID NO: 12 - protein, 15 aa, Artificial Sequence
  Synthetic peptide derived from HER3 protein sequence
IAGLVVIFMMLGGTF

SEQ ID NO: 13 - protein, 11 aa, Artificial Sequence
  Synthetic peptide derived from HER3 protein sequence
LERGESIEPLD

SEQ ID NO: 14 - protein, 11 aa, Artificial Sequence
  Synthetic peptide derived from HER3 protein sequence
QLLYSEAKTPI

SEQ ID NO: 15 - protein, 15 aa, Artificial Sequence
  Synthetic peptide derived from HER3 protein sequence
ESIHFGKYTHQSDVW

SEQ ID NO: 16 - protein, 15 aa, Artificial Sequence
  Synthetic peptide derived from HER3 protein sequence
VWELMTFGAEPYAGL

SEQ ID NO: 17 - protein, 8 aa, Artificial Sequence
  Synthetic peptide derived from HER3 protein sequence
ESGPGIAP

SEQ ID NO: 18 - protein, 15 aa, Artificial Sequence
  Synthetic peptide derived from HER3 protein sequence
TLNRPRGSQSLLSPS

SEQ ID NO: 19 - protein, 15 aa, Artificial Sequence
  Synthetic peptide derived from HER3 protein sequence
EAELQEKVSMCRSRS

SEQ ID NO: 20 - protein, 11 aa, Artificial Sequence
  Synthetic peptide derived from HER3 protein sequence
EEDVNGYVMPD

SEQ ID NO: 21 - protein, 7 aa, Artificial Sequence
  Synthetic peptide derived from HER3 protein sequence
MPTAGTT

SEQ ID NO: 22 - protein, 12 aa, Artificial Sequence
  Synthetic peptide derived from HER3 protein sequence
DGGGPGGDYAAM

SEQ ID NO: 23 - DNA, 35 bp, Artificial Sequence
  Synthetic primer hHER3-F
cagggcggccgcaccatgagggcgaacgacgctct

SEQ ID NO: 24 - DNA, 37 bp, Artificial Sequence
  Synthetic primer hHER3-ECDTM-R
acaagcggccgcagttaaaaagtgccgcccagcatca

SEQ ID NO: 25 - DNA, 39 bp, Artificial Sequence
  Synthetic primer hHER3-ECD-R
acaagcggccgcatttatgtcagatgggttttgccgatc

SEQ ID NO: 26 - DNA, 33 bp, Artificial Sequence
  Synthetic primer hHER3-ECDC1C2-R
acaagcggccgcattgtcagatgggttttgccg

SEQ ID NO: 27 - protein, 9 aa, Artificial Sequence
  Synthetic peptide derived from HER3 protein sequence - HER3 Antigen 1
LAEVPDLLE

SEQ ID NO: 28 - protein, 14 aa, Artificial Sequence
  Synthetic peptide derived from HER3 protein sequence - HER3 Antigen 2
YMVMVKCWMIDENI

SEQ ID NO: 29 - protein, 12 aa, Artificial Sequence
  Synthetic peptide derived from HER3 protein sequence - HER3 Antigen 3
VMVKCWMIDENI

SEQ ID NO: 30 - protein, 9 aa, Artificial Sequence
  Synthetic peptide derived from HER3 protein sequence - HER3 antigen 4
IKVIEDKSG

SEQ ID NO: 31 - protein, 1342 aa, Homo sapiens
  MISC_FEATURE (1)..(1342): Human HER3 Protein Precursor amino acid sequence (GenBank AAA35979.1)
MRANDALQVLGLLFSLARGSEVGNSQAVCPGTLNGLSVTGDAENQYQTLYKLYERCEVVMGNLEIVLTGHNADLSFLQWIREVTGYVLVAMNEFSTLPLPNLRVVRGTQVYDGKFAIFVMLNYNTNSSHALRQLRLTQLTEILSGGVYIEKNDKLCHMDTIDWRDIVRDRDAEIVVKDNGRSCPPCHEVCKGRCWGPGSEDCQTLTKTICAPQCNGHCFGPNPNQCCHDECAGGCSGPQDTDCFACRHFNDSGACVPRCPQPLVYNKLTFQLEPNPHTKYQYGGVCVASCPHNFVVDQTSCVRACPPDKMEVDKNGLKMCEPCGGLCPKACEGTGSGSRFQTVDSSNIDGFVNCTKILGNLDFLITGLNGDPWHKIPALDPEKLNVFRTVREITGYLNIQSWPPHMHNFSVFSNLTTIGGRSLYNRGFSLLIMKNLNVTSLGFRSLKEISAGRIYISANRQLCYHHSLNWTKVLRGPTEERLDIKHNRPRRDCVAEGKVCDPLCSSGGCWGPGPGQCLSCRNYSRGGVCVTHCNFLNGEPREFAHEAECFSCHPECQPMEGTATCNGSGSDTCAQCAHFRDGPHCVSSCPHGVLGAKGPIYKYPDVQNECRPCHENCTQGCKGPELQDCLGQTLVLIGKTHLTMALTVIAGLVVIFMMLGGTFLYWRGRRIQNKRAMRRYLERGESIEPLDPSEKANKVLARIFKETELRKLKVLGSGVFGTVHKGVWIPEGESIKIPVCIKVIEDKSGRQSFQAVTDHMLAIGSLDHAHIVRLLGLCPGSSLQLVTQYLPLGSLLDHVRQHRGALGPQLLLNWGVQIAKGMYYLEEHGMVHRNLAARNVLLKSPSQVQVADFGVADLLPPDDKQLLYSEAKTPIKWMALESIHFGKYTHQSDVWSYGVTVWELMTFGAEPYAGLRLAEVPDLLEKGERLAQPQICTIDVYMVMVKCWMIDENIRPTFKELANEFTRMARDPPRYLVIKRESGPGIAPGPEPHGLTNKKLEEVELEPELDLDLDLEAEEDNLATTTLGSALSLPVGTLNRPRGSQSLLSPSSGYMPMNQGNLGESCQESAVSGSSERCPRPVSLHPMPRGCLASESSEGHVTGSEAELQEKVSMCRSRSRSRSPRPRGDSAYHSQRHSLLTPVTPLSPPGLEEEDVNGYVMPDTHLKGTPSSREGTLSSVGLSSVLGTEEEDEDEEYEYMNRRRRHSPPHPPRPSSLEELGYEYMDVGSDLSASLGSTQSCPLHPVPIMPTAGTTPDEDYEYMNRQRDGGGPGGDYAAMGACPASEQGYEEMRAFQGPGHQAPHVHYARLKTLRSLEATDSAFDNPDYWHSRLFPKANAQRT

SEQ ID NO: 32 - protein, 678 aa, Homo sapiens
  MISC_FEATURE (1)..(678): HER3 intracellular domain (amino acids 665-1342)
YWRGRRIQNKRAMRRYLERGESIEPLDPSEKANKVLARIFKETELRKLKVLGSGVFGTVHKGVWIPEGESIKIPVCIKVIEDKSGRQSFQAVTDHMLAIGSLDHAHIVRLLGLCPGSSLQLVTQYLPLGSLLDHVRQHRGALGPQLLLNWGVQIAKGMYYLEEHGMVHRNLAARNVLLKSPSQVQVADFGVADLLPPDDKQLLYSEAKTPIKWMALESIHFGKYTHQSDVWSYGVTVWELMTFGAEPYAGLRLAEVPDLLEKGERLAQPQICTIDVYMVMVKCWMIDENIRPTFKELANEFTRMARDPPRYLVIKRESGPGIAPGPEPHGLTNKKLEEVELEPELDLDLDLEAEEDNLATTTLGSALSLPVGTLNRPRGSQSLLSPSSGYMPMNQGNLGESCQESAVSGSSERCPRPVSLHPMPRGCLASESSEGHVTGSEAELQEKVSMCRSRSRSRSPRPRGDSAYHSQRHSLLTPVTPLSPPGLEEEDVNGYVMPDTHLKGTPSSREGTLSSVGLSSVLGTEEEDEDEEYEYMNRRRRHSPPHPPRPSSLEELGYEYMDVGSDLSASLGSTQSCPLHPVPIMPTAGTTPDEDYEYMNRQRDGGGPGGDYAAMGACPASEQGYEEMRAFQGPGHQAPHVHYARLKTLRSLEATDSAFDNPDYWHSRLFPKANAQRT

SEQ ID NO: 33 - protein, 214 aa, Homo sapiens
  MISC_FEATURE (1)..(214): Amino acids 741-954 of HER3
IKVIEDKSGRQSFQAVTDHMLAIGSLDHAHIVRLLGLCPGSSLQLVTQYLPLGSLLDHVRQHRGALGPQLLLNWGVQIAKGMYYLEEHGMVHRNLAARNVLLKSPSQVQVADFGVADLLPPDDKQLLYSEAKTPIKWMALESIHFGKYTHQSDVWSYGVTVWELMTFGAEPYAGLRLAEVPDLLEKGERLAQPQICTIDVYMVMVKCWMIDENI

SEQ ID NO: 34 - protein, 664 aa, Homo sapiens
  MISC_FEATURE (1)..(664): HER3 extracellular domain and transmembrane domain
MRANDALQVLGLLFSLARGSEVGNSQAVCPGTLNGLSVTGDAENQYQTLYKLYERCEVVMGNLEIVLTGHNADLSFLQWIREVTGYVLVAMNEFSTLPLPNLRVVRGTQVYDGKFAIFVMLNYNTNSSHALRQLRLTQLTEILSGGVYIEKNDKLCHMDTIDWRDIVRDRDAEIVVKDNGRSCPPCHEVCKGRCWGPGSEDCQTLTKTICAPQCNGHCFGPNPNQCCHDECAGGCSGPQDTDCFACRHFNDSGACVPRCPQPLVYNKLTFQLEPNPHTKYQYGGVCVASCPHNFVVDQTSCVRACPPDKMEVDKNGLKMCEPCGGLCPKACEGTGSGSRFQTVDSSNIDGFVNCTKILGNLDFLITGLNGDPWHKIPALDPEKLNVFRTVREITGYLNIQSWPPHMHNFSVFSNLTTIGGRSLYNRGFSLLIMKNLNVTSLGFRSLKEISAGRIYISANRQLCYHHSLNWTKVLRGPTEERLDIKHNRPRRDCVAEGKVCDPLCSSGGCWGPGPGQCLSCRNYSRGGVCVTHCNFLNGEPREFAHEAECFSCHPECQPMEGTATCNGSGSDTCAQCAHFRDGPHCVSSCPHGVLGAKGPIYKYPDVQNECRPCHENCTQGCKGPELQDCLGQTLVLIGKTHLTMALTVIAGLVVIFMMLGGTFL

SEQ ID NO: 35 - protein, 351 aa, Mus musculus
  MISC_FEATURE (1)..(351): C1C2 domains of mouse lactadherin
TEYICQCPVGYSGIHCETGCSTQLGMEGGAIADSQISASSVYMGFMGLQRWGPELARLYRTGIVNAWTASNYDSKPWIQVNLLRKMRVSGVMTQGASRAGRAEYLKTFKVAYSLDGRKFEFIQDESGGDKEFLGNLDNNSLKVNMFNPTLEAQYIKLYPVSCHRGCTLRFELLGCELHGCSEPLGLKNNTIPDSQMSASSSYKTWNLRAFGWYPHLGRLDNQGKINAWTAQSNSAKEWLQVDLGTQRQVTGIITQGARDFGHIQYVASYKVAHSDDGVQWTVYEEQGSSKVFQGNLDNNSHKKNIFEKPFMARYVRVLPVSWHNRITLRLELLGCFEQKLISEEDLNMHTG

SEQ ID NO: 36 - protein, 24 aa, Mus musculus
  MISC_FEATURE (1)..(24): Leader sequence of mouse lactadherin
MQVSRVLAALCGMLLCASGLFAAS

SEQ ID NO: 37 - protein, 327 aa, Homo sapiens
  MISC_FEATURE (1)..(327): C1C2 domains of human lactadherin
YAGNHCETKCVEPLGMENGNIANSQIAASSVRVTFLGLQHWVPELARLNRAGMVNAWTPSSNDDNPWIQVNLLRRMWVTGVVTQGASRLASHEYLKAFKVAYSLNGHEFDFIHDVNKKHKEFVGNWNKNAVHVNLFETPVEAQYVRLYPTSCHTACTLRFELLGCELNGCANPLGLKNNSIPDKQITASSSYKTWGLHLFSWNPSYARLDKQGNFNAWVAGSYGNDQWLQVDLGSSKEVTGIITQGARNFGSVQFVASYKVAYSNDSANWTEYQDPRTGSSKIFPGNWDNHSHKKNLFETPILARYVRILPVAWHNRIALRLELLGC

SEQ ID NO: 38 - protein, 8 aa, Homo sapiens
  MISC_FEATURE (1)..(8): Leader sequence of human lactadherin
YTCTCLKG

(19) United States
(12) Patent Application Publication
(10) Pub. No.: US 2021/0330773 A1
(43) Pub. Date: Oct. 28, 2021

(54) VACCINES AGAINST ANTIGENS INVOLVED IN THERAPY RESISTANCE AND METHODS OF USING SAME

(71) Applicant: Duke University, Durham, NC (US)

(72) Inventors: Herbert K. Lyerly, Durham, NC (US); Michael A. Morse, Durham, NC (US); Takuya Osada, Durham, NC (US); Timothy M. Clay, Waterloo (BE); Zachary C. Hartman, Durham, NC (US)

(73) Assignee: Duke University, Durham, NC (US)

(21) Appl. No.: 17/328,953

(22) Filed: May 24, 2021

Related U.S. Application Data
(63) Continuation-in-part of application No. 15/962,824, filed on Apr. 25, 2018.
Application No. 15/962,824 is a continuation of application No. 14/373,103, filed on Jul. 18, 2014, now Pat. No. 9,956,276.
Application No. 14/373,103 is a continuation-in-part of application No. 15/942,812, filed on Apr. 2, 2018, now abandoned.
(60) Provisional application No. 61/588,449, filed on Jan. 19, 2012.
(60) Provisional application No. 62/479,870, filed on Mar. 31, 2017.
(60) Provisional application No. 62/622,605, filed on Jan. 26, 2018.

(51) Int. Cl. A61K 39/00; A61K 31/517; C07K 16/32; A61K 39/395; A61K 45/06

(52) U.S. Cl. CPC A61K 39/001106; A61K 31/517; C07K 16/32; A61K 39/3955; A61K 45/06; A61K 2039/5256; A61K 2039/585; A61K 2039/507; C07K 2317/73; C07K 2317/734; C07K 2317/76; C07K 2317/77; A61K 2039/53; A61K 2039/505

(57) ABSTRACT

Methods of reducing the likelihood of a cancer or precancer developing resistance to a cancer therapeutic or prevention agent are provided herein. The methods include administering the cancer therapeutic or prevention agent, such as a checkpoint inhibitor and a vaccine comprising a polynucleotide encoding a polypeptide whose expression or activation is correlated with development of resistance of the cancer or precancer to the cancer therapeutic or prevention agent to a subject. The vaccine may include a polynucleotide encoding a HER3 polypeptide. Methods of using the vaccine including the polynucleotide encoding the HER3 polypeptide to treat a cancer or precancer are also provided.

### CROSS-REFERENCE TO RELATED APPLICATIONS

This patent application is a continuation-in-part of U.S. application Ser. No. 15/962,824, filed Apr. 25, 2018, which is a continuation of U.S. patent application Ser. No. 14/373,103, filed Jul. 18, 2014, now issued as U.S. Pat. No. 9,956,276 on May 1, 2018, which is a national stage filing under 35 U.S.C. 371 of International Application No. PCT/US2013/022396, filed Jan. 21, 2013, which claims the benefit of priority of U.S. Provisional Patent Application No. 61/588,449, filed Jan. 19, 2012, both of which are incorporated herein by reference in their entirety.

This patent application is also a continuation-in-part of U.S. application Ser. No. 15/942,812, filed Apr. 2, 2018, which claims the benefit of priority of U.S. Provisional Patent Application No. 62/479,870, filed on Mar. 31, 2017 and U.S. Provisional Patent Application No. 62/622,605, filed on Jan. 26, 2018, the contents of which are incorporated herein by reference in their entireties.

### STATEMENT REGARDING FEDERALLY SPONSORED RESEARCH

This invention was made with United States government support awarded by the National Cancer Institute grant numbers P50 CA89496-01, P50 CA068438 and R01 CA95447 and by Department of Defense grant numbers BC050221 and W81XWH-12-1-0574. The government has certain rights in this invention.

### SEQUENCE LISTING

This application is being filed electronically via EFS-Web and includes an electronically submitted Sequence Listing in .txt format. The .txt file contains a sequence listing entitled “155554_00609_ST25.txt” created on May 24, 2021 and is 99,846 bytes in size. The Sequence Listing contained in this .txt file is part of the specification and is hereby incorporated by reference herein in its entirety.

### INTRODUCTION

This application relates to a cancer vaccine, specifically a vaccine against antigens that are expressed in response to resistance to therapeutic intervention to cancer (or pre-cancers), with a proof of principle antigen, HER3, as an example. Methods of using the vaccines and methods of developing vaccines capable of blocking the development of resistance to cancer therapies are also provided.

Cancer vaccines target antigens expressed by tumors, but application of these vaccines has not been as effective as once hoped due to induction of immune tolerance by chronic overexpression of the targeted protein in the absence of co-stimulatory molecules and the induction of an immunomodulatory environment. Preventative cancer vaccines may be more promising, but cancers are highly variable, with multiple genetic changes, but few truly universal changes. Thus, it is difficult to predict what antigens will be overexpressed on any specific cancer or whether an individual should be vaccinated and if so, with what antigens. In contrast, a strategy is proposed here in which vaccination against the antigen(s) that will predictably be overexpressed in response to a therapy, but prior to that antigen's over-expression by the cancer cells is used to induce a robust anti-cancer immune response.

The human epidermal growth factor receptor (HER) family, consisting of HER1 (also known as EGFR), HER2, HER3 and HER4, drives the progression of many epithelial malignancies. EGFR and HER2 have been extensively studied as mediators of poor prognosis and are credentialed therapeutic targets of both small molecule inhibitors and monoclonal antibody therapy. In contrast, HER3, overexpressed in breast, lung, gastric, head and neck, and ovarian cancers and melanoma, is associated with poor prognosis, but has not been a credentialed therapeutic target because it lacks catalytic kinase activity and is not transforming by itself. However, HER3 is thought to function as a signaling substrate for other HER proteins with which it heterodimerizes (13, 14). Not only are these HER3 heterodimers potent oncogenic signaling drivers, but also they have been described as a cause of therapeutic resistance to anti-EGFR, anti-HER2 and hormonal therapies. Therefore, HER3 is an attractive therapeutic target. Although the lack of a catalytic kinase domain limits direct inhibition with small molecule tyrosine kinase inhibitors (TKIs), HER3 may be targeted with antibodies that either block binding of its ligand neuregulin-1 (NRG-1) (also called heregulin) or cause internalization of HER3, inhibiting downstream signaling. Additionally, the anti-HER2 monoclonal antibody pertuzumab disrupts neuregulin-induced HER2-HER3 dimerization and signaling; however, it is less effective at disrupting the elevated basal state of ligand-independent HER2-HER3 interaction and signaling in HER2-overexpressing tumor cells. There, however, remains a need in the art for therapeutic alternatives to monoclonal antibodies that may target the HER3 protein.

### SUMMARY

Provided herein is a mechanism of revolutionizing cancer therapy or prevention by preventing the development of resistance to cancer therapeutic or cancer prevention agents, including checkpoint inhibitors, by identifying which antigens are likely to be expressed in a cancer or precancer in response to treatment with a cancer therapeutic or prevention agent and thus which antigens may be targeted with a vaccine in patients. Also provided is a vaccine targeting a specific antigen involved in a resistance mechanism, namely HER3, and methods of using the vaccine. In one aspect, the vaccine includes a polynucleotide encoding a HER3 polypeptide. For example, a HER3 polypeptide of SEQ ID NO: 1 or 2 may be included in a vaccine. A polynucleotide encoding the HER3 polypeptide may include a polypeptide having at least 60%, 70%, 80%, 85%, 90%, 92%, 95%, 96%, 97%, 98%, or 99%, or 100% sequence identity to SEQ ID NO: 1 (Human HER3 Protein amino acid sequence), SEQ ID NO: 2 (Human HER3 Protein Precursor amino acid sequence), or any one of SEQ ID NOS: 5-22 or 27-34 (HER3 Antigenic Epitopes).

In another aspect, compositions including any one of the vaccine vectors described herein and a checkpoint inhibitor are provided.

In a further aspect, pharmaceutical compositions are provided. The pharmaceutical compositions may include a pharmaceutically-acceptable carrier and any one of the vaccine vectors described herein or any one of the combination compositions described herein.

In another aspect, methods of treating a cancer or precancer or reducing the likelihood of the cancer or precancer to develop resistance to a cancer therapeutic or prevention agent by administering the vaccine provided herein to a subject with cancer or precancer are provided. The vaccine may be administered before, concurrently with or after administration of the cancer therapeutic or prevention agent.

In yet another aspect, methods of reducing the likelihood of a cancer or precancer developing resistance to a cancer therapeutic or prevention agent by administering the cancer therapeutic or prevention agent and a vaccine to the subject are provided. The vaccine includes a polynucleotide encoding a polypeptide whose expression or activation correlates with development of resistance of the cancer or precancer to the cancer therapeutic or prevention agent. Co-administration of the cancer therapeutic or prevention agent and the vaccine inhibits the generation of resistance to the cancer therapeutic or prevention agent and increases the therapeutic potential of the cancer therapeutic agent and the prevention potential of the cancer prevention agent.

In another aspect, the methods may include administering a therapeutically effective amount of any one of the vaccine vectors described herein to the subject having the cancer or precancer, and administering a therapeutically effective amount of a checkpoint inhibitor. Optionally, each of these methods may further include administering a therapeutically effective amount of an additional cancer therapeutic or prevention agent to the subject.

### BRIEF DESCRIPTION OF THE DRAWINGS

FIG. 1 is a set of figures showing HER3 specific T cell and B cell responses to Ad-HER3 in vivo. FIG. 1A is a graph showing the number of IFN-γ secreting splenocytes by ELISPOT after 6-8 week old BALB/c mice were immunized once with 2.6×10¹⁰ Ad-HER3 or Ad-GFP via bilateral subcutaneous footpad injections. Two weeks following the vaccination mice were euthanized and splenocytes collected for analysis in an Interferon-gamma ELISPOT assay. Splenocytes from Ad-HER3 vaccinated and not or Ad-GFP vaccinated (control) mice recognized HER3 intracellular domain (ICD) and extracellular domain (ECD) peptide libraries and the mixture of both libraries (Mix) in interferon-gamma ELISPOT assays. The mean from 5 mice per group is shown with error bars denoting standard deviation. CT−; splenocytes alone. CT+; Splenocytes plus PMA (50 ng/mL) and Ionomycin (1 ng/mL) as a control for the assay. FIG. 1B is a set of FACS analysis histograms of peripheral blood serum from the mice was tested for the presence of antibodies capable of binding to tumor cell-expressed HER3. Flow cytometric analysis was used and histograms denote binding of HER3-vaccine induced antibodies (HER3-VIA) in serum to human breast cancer cell line BT474. FIG. 1C is a graph showing the mean fluorescence intensity which was calculated for the binding of HER3-VIA against a panel of human breast cancer cell lines with dilutions of the serum. FIG. 1D shows the results of epitope mapping of HER3-VIA using spotted 15mer peptide arrays and revealed recognition of 18 different epitopes.

FIG. 2 is a set of figures showing that HER3-VIA mediate multiple mechanisms of action on human breast tumor cell lines in vitro. FIG. 2A is a set of graphs showing that HER-3 VIA mediate complement dependent cytotoxicity (CDC) against HER3-expressing (BT474, T47D, MDA-MB-468, BT474M1) human breast cancer cell lines but not against the HER3-negative cell line (MDA-MB-231). Black bars, HER3-VIA; white bars, GFP-VIA; grey bars, Trastuzumab. Trastuzumab does not mediate CDC. FIG. 2B is a graph showing that HER-3 VIA mediate antiproliferative activity against HER3-expressing (BT474, T47D, MDA-MB-468, BT474M1) human breast cancer cell lines but not against the HER3-negative cell line (MDA-MB-231) in a 72 hour assay. The antiproliferative effect implied receptor modulation and FIG. 2C is a set of photographs showing that binding of HER3-VIA results in rapid internalization of endogenous HER3 receptor expressed on the surface of human breast cancer cell lines.

FIG. 3 is a set of figures showing the in vivo effects of HER3-VIA on BT474M1 human breast tumor xenografts. FIG. 3A is a cartoon showing the experiment schema. HER3-VIA or control GFP-VIA were transferred via tail vein injections. FIG. 3B is a graph showing that HER3-VIA retarded the growth of established BT474M1 breast cancers (p<0.005 at *) FIG. 3C is a set of photographs showing immunohistochemistry analysis of HER3 protein expression in excised tumors and revealed a dramatic loss of HER3 protein in the HER3-VIA-treated mice compared to GFP-VIA treated mice. GFP-VIA-treated mouse tumors retained HER3 protein levels seen in tumors from mice “treated” with saline. FIG. 3D is a set of photographs of Western blot analysis of excised tumors for expression of the indicated proteins.

FIG. 4 is a set of figures showing the in vivo effects of HER3-VIA in lapatinib-refractory rBT474 SCID tumor xenografts. FIG. 4A is a graph showing that passive transfer of HER3-VIA retarded the growth of established lapatinib-refractory BT474 tumors in SCID mice demonstrating that anti-HER3 immunity can treat therapy resistant tumors (p<0.025 at *). FIG. 4B is a set of photographs showing Western blot analysis of excised tumors to perform pathway analysis. FIG. 4C is a set of photographs showing immunohistochemical analysis of excised tumors and revealed no significant change in HER3 levels compared to controls.

FIG. 5 is a schematic representation of the primer binding sites on the human Her3 full length cDNA.

FIG. 6 is a graph showing that Ad-HER3 vaccine inhibits JC-HER3 tumor growth. Balb/c mice were vaccinated twice (day-18, day-4) via footpad injection with Ad-GFP or Ad-hHER3 vectors (2.6×10¹⁰ particles/mouse). Four days after boosting, at day 0, each mouse was implanted with 1,000,000 JC-HER3 mouse mammary tumor cells expressing human HER3. Tumor volume was measured, once it became palpable, every 3 days using calipers and is reported.

FIG. 7 is a graph showing Ad-hHER3 vaccine induced HER3 specific T cell response. Splenocytes (500,000 cells/well) from vaccinated Balb/c mice in FIG. 6 (x-axis) were collected at day 28 and stimulated with HER3 peptide mix (hHER3 peptides) (1 μg/mL was used; JPT, Acton, Mass.) or HIV peptide mix (BD Bioscience) as a negative control (Negative CT) and analyzed in a interferon-gamma ELISpot assay.

FIG. 8 is a set of photographs showing that Ad-hHER3 vaccination causes degradation of HER3 on JC-hHER3 tumor. Tumors were isolated from vaccinated and control Balb/c mice (as indicated on figure) and immediately flash frozen. Tissue extracts were prepared by homogenization in RIPA buffer. Equal amounts of protein from each sample were used to visualize the indicated molecules by immunoblotting.

FIG. 9 is a set of FACS histograms showing that Ad-hHER3 vaccination decreases HER3 expression on JC-hHER3 tumor cells. JC-HER3 tumors were collected from vaccinated and control Balb/c mice (as indicated on figure) at day 28 and pooled by group. The tissues were minced and digested with an enzymatic cocktail (Hyaluronalse, DNAse, and Collagenase) overnight. After 3 days culture, the cells were harvested and HER3 expression determined by flow cytometry using PE-anti-hHER3 antibody.

FIGS. 10A-10D show combined JC-HER3 tumor growth and mouse survival data following treatment with Ad[E1-E2b-]HER3 vaccine. FIG. 10A is a graph showing the antitumor effect after JC-HER3 tumor cells were implanted in HER3-transgenic F1 hybrid mice (5×10⁵ cells/mouse) and mice were immunized on days 3 and 10 with Ad-HER3-FL (SEQ ID NO: 2), Ad[E1-E2b-]GFP (2.6×10E10 vp/injection) or saline. The longitudinal mixed effects model with the maximum likelihood variance estimation method was used to model tumor volume over time. Mean±SE is shown. (Ad-HER3 FL and Ad-GFP: 15 mice / group, saline: 10 mice) *p<0.001 FIG. 10B is a graph showing the effect of Ad[E1-E2b-]HER3-FL vaccine on mouse survival. JC-HER3 tumor cells were implanted in HER3-transgenic F1 hybrid mice and immunized as above in FIG. 10A. Mice were considered censored at the time the tumor volume reached humane endpoint and were euthanized. The Kaplan-Meier method was used to estimate overall survival and treatments were compared using a two-sided log-rank test. FIG. 10C is a blot showing the effect of Ad-HER3 vaccine on HER3 expression by JC-HER3 tumors. When tumor volume reached humane endpoint, mice were sacrificed and tumor tissues were collected. Western blot was performed with anti-hHER3 antibody (Santa Cruz), followed by HRP-conjugated anti-mouse IgG (Cell Signaling) and chemiluminescent development. FIG. 10D is a set of plots showing the effect of Ad-HER3 vaccine on HER3 expression by flow cytometry. JC-HER3 tumors were collected and digested after a vaccine prevention model experiment and pooled by group. hHER3 expression was determined by FACS using PE-anti-hHER3 antibody. Open histograms show HER3 expression, and grey filled histograms show the staining with PE-conjugated isotype control.

FIGS. 11A-11E show analysis of tumor-infiltrating T cells in comparison with splenocytes and lymph node cells. HER3-transgenic mice bearing JC-HER3 tumor and immunized with either Ad-HER3-FL or Ad-GFP were euthanized, and tumors, spleen and lymph nodes were collected from each mouse. Tumors were digested and tumor cells were stained with viability dye and anti-CD3, CD4, CD8, PD-1 and PD-L1 antibodies and analyzed by flow cytometry. FIG. 11A is a graph showing CD3+ T cells as a percentage of total cells in the tumor digest. Percentage of T cells from the tumor of each mouse. Bars show the mean. FIG. 11B is a set of graphs showing CD4 and CD8 T cell population in tumors, spleen, and lymph nodes. Bars represent mean+/−SD percentages of CD4+ and CD8+ cells in CD3+ T cell population for each site. *p<0.05. FIG. 11C is a set of graphs showing CD25+FOXP3 cells in tumor, spleens, and lymph nodes. Bars represent mean+/−SD percentages of CD25+FOXP3+cells in CD4+T cell population for each site. Student's T test: * p=0.026 and ** p=0.008. FIG. 11D is a set of graphs showing PD-1 expression by T cells in tumors, spleens, lymph nodes and tumors. CD4+ and CD8+ T cells from each site were analyzed for their expression of PD-1 by flow cytometry. Bars represent mean+/−SD for n=3 mice. FIG. 11E is a graph showing PD-L1 expression by tumor cells after Ad-HER3-FL or Ad-GFP vaccination. Expression of PD-L1 by tumor cells was analyzed for each mouse treated with Ad-HER3-FL or Ad-GFP vaccine and shown as percentage. Bars show the mean.

FIG. 12 shows the antitumor effect of Ad-HER3-FL vaccine and PD-1/PD-L1 blockade in HER3 transgenic mice bearing JC-HER3 tumors. Tumor growth inhibition in a prevention model. HER3-transgenic mice were vaccinated with Ad-HER3-FL (2.6×10¹⁰ vp/mouse) on days -11 and -4 and then implanted with JC-HER3 cells (0.5×10⁶ cells/mouse) in the flank on day 0. Mice received intraperitoneal injections of anti-PD-1 or anti-PD-L1 antibody (200 μg/injection) on days 3, 6, 10, 13, 17, and 20. Mean±2SE is shown. *p<0.05, **p<0.01, ***p<0.001

FIGS. 13A-13B show enhanced T cell infiltration into JC-HER3 tumors in mice treated with Ad-HER3-FL vaccine and PD-1/PD-L1 blockade. JC-HER3 tumors from mice immunized with HER3 or control vaccine and treated with/without PD-1/PD-L1 blockade were analyzed for CD3+ T cell infiltration by immunohistochemistry. FIG. 13A is a set of photographs showing increased CD3+ T cell infiltration with Ad-HER3-FL and anti-PD-1 therapy. High power fields were selected randomly at magnification of ×200, and 10 fields that did not include necrotic area were evaluated. Representative high power fields of tumor sections for each group are shown. FIG. 13B is a graph showing the highest CD3+ T cell infiltration was obtained with a combination of Ad-HER3-FL and anti-PD-1 therapy. Two independent observers counted the number of CD3+ T cells in the fields, and the average of 10 fields for each group were shown. Error Bar: SD. *p<0.05, **p<0.01, ***p<0.0001.

FIGS. 14A-14B show immune responses induced by combination of Ad-HER3-FL vaccine and PD-1/PD-L1 blockade. FIG. 14A is a graph showing HER3-specific Cellular Immune Response. HER3-transgenic mice were immunized with Ad-HER3-FL or Ad-GFP and tumor was implanted followed by anti-PD-1 or anti-PD-L1 antibody therapy. At day 25, when mice were euthanized, an IFN-gamma ELISPOT assay was performed with splenocytes from individual mice (n=3 mice per group). HER3 ECD, HER3 ICD and ECD+ ICD peptide pool were used as stimulating antigens. Bars represent the number of spots (representing IFN-gamma secreting T cells)+/−SD. P-value: *p<0.05, **p<0.01, ***p<0.001. FIG. 14B is a graph showing the anti-HER3 Humoral Immune Response. When mice were euthanized on day 25, blood was collected from individual mice (n=3 mice per group), and a cell-based ELISA was performed using the serum. Sera from immunized mice were applied at serial dilutions of 1:50 to 1:6400. nIR-conjugated secondary antibody was added at 1:2000 dilution. nIR signals were detected by the LI-COR Odyssey imager at 700 nm channel. The average of difference of nIR signals between the 4T1-HER3 wells and 4T1 wells are shown.

FIG. 15 shows tumor growth inhibition improved with sequential Ad-HER3 vaccination followed by immune checkpoint blockade. HER3 transgenic mice were implanted with JC-HER3 cells (0.5×10⁶ cells/mouse) in the flank on day 0, then vaccinated with Ad-HER3-FL or control Ad-GFP (2.6×10¹⁰ vp/mouse) on days 3 and 10. Mice received intraperitoneal injection of anti-PD-L1 antibody and/or anti-CTLA4 antibody or control IgG (200 μg/injection) twice a week (on days 3, 7, 10, 14, 17 and 21). Mean±2SE is shown. *p<0.005, **p<0.001.

FIGS. 16A-16B show immune responses induced by combination of Ad-HER3-FL vaccine and either PD-1/PD-L1 blockade or CTLA4 blockade. FIG. 16A is a graph showing anti-HER3 Cellular Immune Response: IFN-gamma ELISPOT assay was performed using splenocytes collected at the euthanasia of mice. HER3 ECD (SEQ ID NO: 34), HER3 ICD (SEQ ID NO: 32) or mixture of ECD and ICD peptide pool (SEQ ID NOs: 5-22 and 27-34) were used as stimulating antigens. HIV peptide pool was used as negative control. Numbers of spots in medium alone (no stimulating antigen) were subtracted and shown. Error bars: SD. *p<0.05, **p<0.01, ***p<0.005. FIG. 16B is a graph showing anti-HER3 Humoral Immune Response: Cell-base ELISA for anti-HER3 antibody was performed using mouse serum collected at the euthanasia of mice. Titrated mouse sera were added to 4T1 cell-coated 96-well plate or 4T1-HER3 cell-coated 96-well plate. After incubation, nIR-conjugated anti-mouse IgG antibody was added. nIR signals were detected by the LI-COR Odyssey imager at 700 nm channel. The average of difference of nIR signals between the 4T1-HER3 wells and 4T1 wells are shown.

FIGS. 17A-17D show the effect of Ad-HER3-FL vaccine and checkpoint inhibitors on T cell subpopulations in Spleens and Tumors of vaccinated mice. FIG. 17A is a graph showing tumor infiltrating lymphocytes (TIL) were isolated from tumor tissues. Percentages of CD25+ Foxp3+ in CD4 T cells in TILs. FIG. 17B is a graph showing the spleens T cell numbers after spleens were harvested and analyzed by flow cytometry assay. Percentages of CD25+ Foxp3+ in CD4 T cells in splenocytes. FIG. 17C is a graph showing the CD8/Treg ratio in Tumor infiltrating lymphocytes (TIL). FIG. 17D is a graph showing the CD8/Treg ratio in splenocytes. *p<0.05, **p<0.01, ***p<0.001.

FIGS. 18A-18B show anti-HER3 immune response induced by Ad-HER3-FL vaccination in human HER3-transgenic mice. FIG. 18A is a graph showing the cellular immune response. Human HER3-transgenic mice were vaccinated at 0 and 14 days with Ad-HER3-FL, control Ad-GFP (2.6×10¹⁰ vp/vaccination), or saline. The mice were sacrificed on day 21 and splenocytes were harvested. IFN-g ELISPOT assays were performed with splenocytes using peptide pools derived from HER3-ECD, HER3-ICD, or HIV (negative control) as stimulating antigens. The number of spots indicating T cells secreting IFN-g in response to the respective peptide pools is reported. Average values of 4 mice from each group are shown. P-value: *p<0.0001. FIG. 18B is a graph showing the humoral immune response. 4T1 and 4T1-HER3 cells seeded into 96 well flat-bottomed plates the day prior were incubated for 1 h on ice with serum (at serial dilutions of 1:50 to 1:6400) from 4 mice vaccinated as in FIG. 18A and collected at the time of sacrifice. The cells were then fixed with 1% formaldehyde and HRP-labeled Goat anti-mouse IgG (1:2000) was added. After 1 h incubation, TMB was added for 5 min for color development and H₂SO₄ was added to stop the reaction. The average differences of OD450 values ([value for 4T1-HER3]-[value for 4T1]) are shown.

### DETAILED DESCRIPTION

As a novel alternative to vaccines targeting well established tumor antigens, we hypothesized that the antigen-specific immune non-responsiveness to conventional tumor-associated antigens may be avoided by targeting tumor antigens that are induced after exposure to a cancer therapeutic or prevention agent as a mechanism of developing therapeutic resistance. Although there may be many potential antigens overexpressed in response to a cancer therapeutic or prevention agent, those antigens that are likely critical components of specific therapeutic resistance mechanisms would be attractive targets, as immunologic ablation of clones expressing such antigens should eliminate the clinical recurrence of therapy resistant tumor cells. One such antigen thought to be essential to therapeutic resistance is a member of the HER family of receptor tyrosine kinases (RTKs), and to endocrine therapies, HER3.

HER3, although lacking catalytic kinase activity, is thought to function as a signaling substrate for other HER proteins with which it heterodimerizes. Although not transforming by itself, HER3 has tumor promoting functions in some cancers, including a role as a co-receptor for amplified HER2 with which it is synergistically co-transforming and rate-limiting for transformed growth. Treatment of HER2-amplified breast cancers with HER2-targeting tyrosine kinase inhibitors (TKIs) leads to an increase in HER3 expression and downstream signaling that results in therapeutic resistance.

The pivotal role of HER3 as a hub for HER family signaling has made it an attractive therapeutic target, but its' lack of kinase activity prevents small molecule HER3 specific TKIs from being generated. Nonetheless, HER3 may be targeted with antibodies which have diverse functional consequences depending on their binding site. For example, the anti-HER2 monoclonal antibody pertuzumab disrupts neuregulin-induced HER2-HER3 dimerization and signaling; however, it is less effective at disrupting the elevated basal state of ligand-independent HER2-HER3 interaction and signaling in HER2-overexpressing tumor cells. Other HER3-specific antibodies under development bind to, and cause internalization of, HER3, inhibiting downstream signaling. As an alternative to monoclonal antibodies, we have recently demonstrated that polyclonal antibodies induced by vaccination against receptors such as HER2 can mediate profound receptor internalization and degradation, providing a therapeutic effect in vitro and in vivo (Ren et al., Breast cancer Research 2012 14: R89).

Therefore, we generated a recombinant adenoviral vector expressing human HER3 (Ad-HER3) and demonstrated that it elicited HER3 specific B and T cell immune responses as shown in the Examples. Furthermore, we demonstrated that HER3 specific antibodies recognized multiple HER3 epitopes, bound to tumor membrane expressed HER3, mediated complement dependent lysis and altered downstream signaling mediated by receptor heterodimers involving HER3. In addition, we found that HER3 specific polyclonal antisera had specific activity in mediating HER3 internalization and degradation. Finally, we demonstrated that HER3 specific polyclonal antisera was well tolerated when transferred to tumor bearing animals, yet retarded tumor growth in vivo, including retarding the growth of HER2 therapy-resistant tumors. These data suggest that Ad-HER3 is an effective vaccine which should be tested for therapeutic efficacy in clinical trials targeting cancers that overexpress HER3 in response to a targeted therapy. The general application of this vaccination strategy can be applied to other antigens expressed in HER therapy resistant tumors, as well as antigens induced by other resistance mechanisms, and represents a new conceptual framework for cancer immunotherapy.

As described in the appended examples, generation of resistance to cancer therapeutic or prevention agents is a common problem in the treatment of cancer or precancer and in several cases the mechanism of resistance to the therapeutic agent is known. Resistance is often the result of changes in gene expression (over-expression or blocked expression of a protein), change in the gene by mutation, or altered sequences by altered splicing or translocation or altered activation of a protein in the cells (over-activation or blocked activation of a protein).

In those cases where over-expression or over-activation of a protein, or a new sequence in the protein is responsible for increasing the resistance of the cancer or precancer cells to the therapeutic or prevention agent, we report a method for reducing the likelihood that the cancer or precancer will develop resistance to the cancer therapeutic or prevention agent. As used herein, resistance to a cancer therapeutic or prevention agent indicates that the cancer therapeutic or prevention agent is not as effective at inhibiting the growth of, or killing, cancer or precancer cells in response to the cancer therapeutic or prevention agent. The method may even block the development of resistance to the cancer therapeutic or prevention agent or may reverse resistance to the cancer therapeutic or prevention agent after it has developed. The methods include administering the cancer therapeutic or prevention agent and administering a vaccine to the subject in need of treatment for a cancer. The vaccine comprises a polynucleotide encoding a polypeptide whose expression or activation is correlated with or results in development of resistance of the cancer or precancer to the cancer therapeutic or prevention agent.

The present inventors hypothesized that activation of T cells by a vaccine against a tumor antigen would lead to increased tumor infiltration of antigen-specific T cells and the anti-tumor activity of these T cells would be enhanced by checkpoint blockade.

In order to activate immune responses against HER3, the present inventors, in the non-limiting Examples, generated a recombinant adenoviral vector expressing full length human HER3 (SEQ ID NO: 2; Ad-HER3-FL) and demonstrated that it elicited HER3-specific humoral and cellular immune responses in HER3-transgenic mice, thus breaking tolerance. They also developed breast cancer models expressing HER3 and surprisingly demonstrated that delayed tumor progression with preventive and therapeutic vaccination was associated with an accumulation of PD-1 expressing-tumor infiltrating lymphocytes (TIL). A combination of the Ad-HER3 vaccine with either anti-PD-1 or anti-PD-L1 antibodies suppressed or eliminated HER3-expressing breast cancer more effectively than either alone when used in preventive models, but had only a modest anti-tumor effect in therapeutic models. A combination of anti-CTLA4 and Ad-HER3 vaccine demonstrated a greater anti-tumor effect in the therapeutic model.

Expression of human epidermal growth factor family member 3 (HER3), a critical heterodimerization partner with EGFR and HER2, promotes more aggressive biology in breast and other epithelial malignancies. As such, inhibiting HER3 could have broad applicability to the treatment of EGFR- and HER2-driven tumors. Although lack of a functional kinase domain limits use of receptor tyrosine kinase inhibitors, HER3 contains antigenic targets for T cells and antibodies. Using novel human HER3 transgenic mouse models of breast cancer, the present inventors demonstrate that immunization with recombinant adenoviral vectors encoding full length human HER3 (Ad-HER3-FL) induces HER3-specific T cells and antibodies, alters the T cell infiltrate in tumors, and influences responses to immune checkpoint inhibitions. Both preventative and therapeutic Ad-HER3-FL immunization delayed tumor growth, but were associated with both intratumoral PD-1 expressing CD8+ T cells and regulatory CD4+ T cell infiltrates. Immune checkpoint inhibition with either anti-PD-1, anti-PD-L1 antibodies increased intratumoral CD8+ T cell infiltration and eliminated tumor following preventive vaccination with Ad-HER3-FL vaccine. The combination of dual PD-1/PD-L1 and CTLA4 blockade slowed the growth of tumor in response to Ad-HER3-FL in the therapeutic model. The present inventors conclude that HER3 -targeting vaccines activate HER3-specific T cells and induce anti-HER3 specific antibodies, which alters the intratumoral T cell infiltrate and responses to immune checkpoint inhibition.

### Abbreviations

The following abbreviations are used throughout this specification:

Ad Adenovirus

CTLA4 Cytotoxic T-Lymphocyte-Associated Protein 4

ECD Extracellular domain

EGFR Epidermal Growth Factor Receptor

ELISA Enzyme-Linked Immunosorbent Assay

ELISPOT Enzyme-Linked ImmunoSpot

FL Full length

HER3 Human Epidermal Growth Factor Receptor 3

HER2 Human Epidermal Growth Factor Receptor 2

ICD Intracellular domain

IHC Immunohistochemistry

OS Overall survival

PD-1 Programmed Death Receptor 1

PD-L 1 Programmed Death Receptor Ligand 1

TIL Tumor infiltrating lymphocytes

As used herein, the terms “protein” or “polypeptide” or “peptide” may be used interchangeably to refer to a polymer of amino acids. A “polypeptide” as contemplated herein typically comprises a polymer of naturally occurring amino acids (e.g., alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine). The proteins contemplated herein may be further modified in vitro or in vivo to include non-amino acid moieties.

The HER3 polypeptides disclosed herein may include “variant” HER3 polypeptides. As used herein the term “wild-type” is a term of the art understood by skilled persons and means the typical form of an organism, strain, gene or characteristic as it occurs in nature as distinguished from variant forms. As used herein, a “variant, “mutant,” or “derivative” refers to a polypeptide molecule having an amino acid sequence that differs from a reference protein or polypeptide molecule. A variant or mutant may have one or more insertions, deletions, or substitutions of an amino acid residue relative to a reference molecule. A variant or mutant may include a fragment of a reference molecule. For example, a HER3 variant molecule may have one or more insertions, deletions, or substitution of at least one amino acid residue relative to the HER3 “wild-type” polypeptide sequence of a particular organism. The polypeptide sequences of the “wild-type” HER3 polypeptides from, for example, humans are presented as SEQ ID NOS: 1-22 and 27-34. The full length HER3 polypeptide is presented as SEQ ID NO: 1 or 2. These sequences may be used as reference sequences.

The vaccine may be administered before, during or after treatment with the cancer therapeutic or prevention agent or may be administered simultaneously with the cancer therapeutic or prevention agent. The administration of the vaccine and the cancer therapeutic or prevention agent to the subject reduces the likelihood that the subject's cancer or precancer will develop resistance to the therapeutic or prevention agent as compared to a control subject with a similar cancer or precancer not administered the vaccine or as compared to the general likelihood of a population of subjects having the cancer or precancer. In some embodiments, the cancer or precancer in individuals administered both the vaccine and the therapeutic or prevention agent does not develop resistance to the cancer therapeutic or prevention agent and is treated. Alternatively, the growth of the cancer or precancer may be inhibited or the growth rate reduced. The administration of the vaccine and cancer therapeutic or prevention agent may also reverse resistance to the cancer therapeutic or prevention agent if the cancer or precancer is already resistant to the cancer therapeutic or prevention agent. In some embodiments, administration of the vaccine is sufficient to treat the cancer or inhibit the growth or kill the cancer. In other embodiments, the vaccine must be administered in conjunction with the cancer therapeutic or prevention agent or prior to development of resistance to the cancer therapeutic or prevention agent by the cancer.

### Vaccine Vectors

The vaccine may include a polynucleotide encoding a HER3 polypeptide. The mature HER3 protein sequence is provided in SEQ ID NO: 1 and the complete HER3 protein precursor sequence is provided in SEQ ID NO: 2. Polynucleotide sequences for HER3 are provided in SEQ ID NO:3 (mRNA) and SEQ ID NO: 4 (DNA). The vaccine may comprise full-length HER3 or portions thereof. For example, the vaccine may comprise only the extracellular domain or the extracellular domain plus the ransmembrane domain or other portions of the HER3 polypeptide. Suitably the vaccine is capable of eliciting an immune response to HER3 in a subject administered the vaccine. The immune response may be a B cell or T cell response. Suitably the immune response includes an antibody response directed to HER3. The immune response may be a polyclonal antibody response in which multiple epitopes of HER3 are recognized by antibodies.

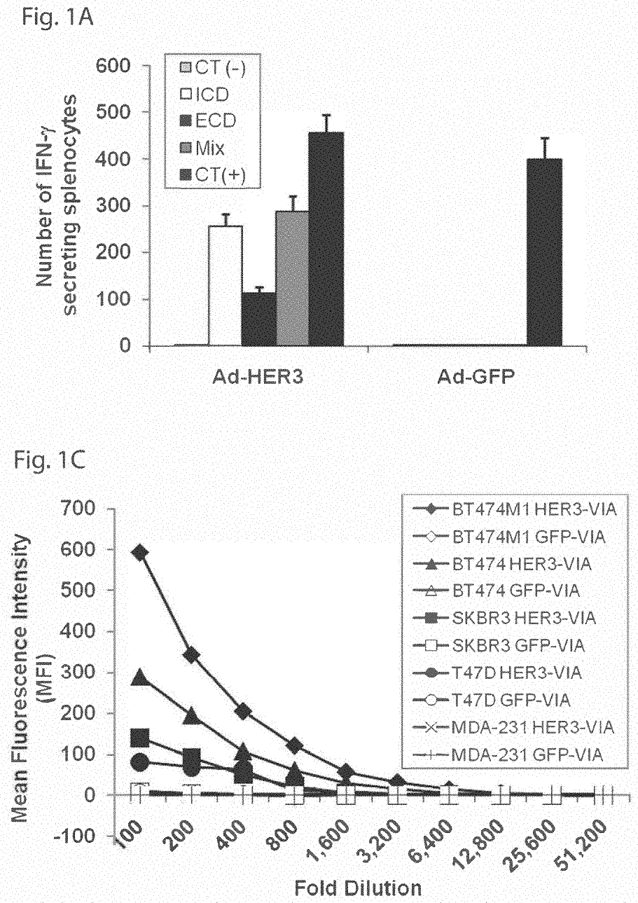

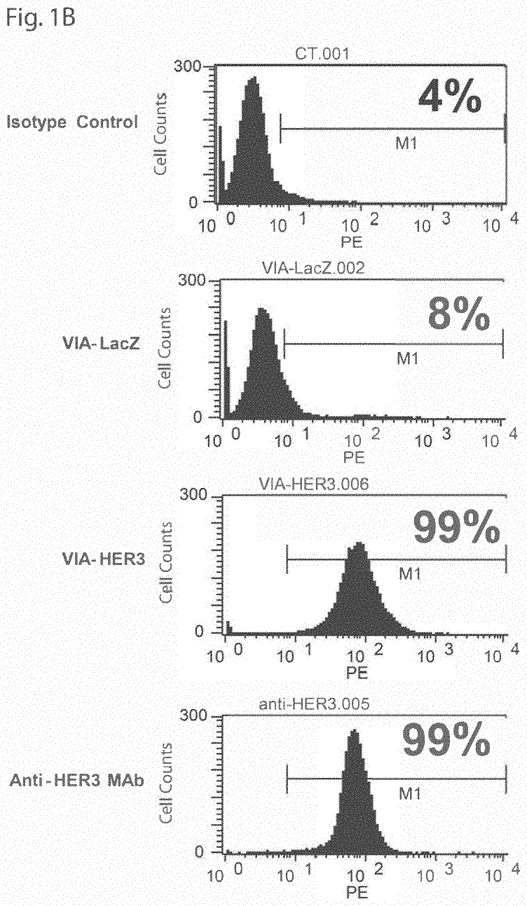

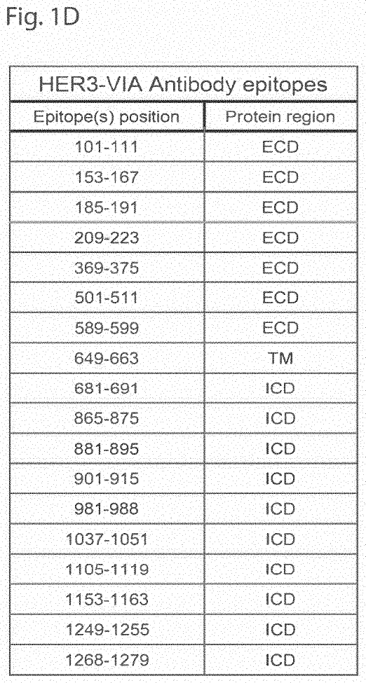

As reported in the examples, in a mouse model a HER3 vaccine was able to generate a robust polyclonal antibody response to HER3 and several epitopes were identified. See FIG. 1D. The epitopes identified in FIG. 1D include the polypeptides identified in SEQ ID NOs: 5-22, which represents portions of SEQ ID NO:2. The epitopes of SEQ ID NO: 27-34 may also be used. The HER3 polypeptide may include a polypeptide having at least 60%, 70%, 80%, 85%, 90%, 92%, 95%, 96%, 97%, 98%, or 99%, or 100% sequence identity to any one of SEQ ID NO: 1-22 or 27-34. In some embodiments the polynucleotides encoding these antigens are used in a vaccine formulation. It is expected that some of these epitopes may be immunogenic in humans as well. Those of skill in the art will appreciate that a vaccine including polynucleotides encoding only portions of full-length HER3, i.e. antigenic epitopes, may be used in the vaccines described herein.

The HER3 polypeptides provided herein may be full-length polypeptides (as in SEQ ID NOS: 1 or 2) or may be fragments of the full-length polypeptide (e.g., SEQ ID NO: 5-22 or 27-34). The HER3 polypeptides may be encompassed in a fragment of full-length HER3. For example, the HER3 polypeptides are all within the intracellular domain of HER3 which is presented as SEQ ID NO: 32. As used herein, a “fragment” is a portion of an amino acid sequence which is identical in sequence to but shorter in length than a reference sequence. A fragment may comprise or consist of up to the entire length of the reference sequence (e.g., SEQ ID NOS: 1-22, 27-34), minus at least one amino acid residue. In some embodiments, a fragment of the HER3 polypeptides may comprise or consist of at least 5, 6, 7, 8, 9, or more amino acids thereof. Preferably, a fragment of a HER3 antigenic polypeptide includes the amino acid residues responsible for eliciting an immune response such as a T cell response in a subject.

The vaccine may include a vaccine vector. The vaccine vector may be a bacterial, yeast, viral or liposomal vaccine vector. The vaccine may be a DNA or polynucleotide based vaccine as well and not include a vaccine vector. The vaccine vector may be an adenovirus or adeno-associated virus. In the Examples an adenovirus was used as the vaccine vector. The vaccine vector may contain the HER3 polynucleotide or portions thereof. The vaccine vector may contain the HER3 polypeptide or portions thereof. The vaccine vector may express the HER3 polypeptide or portions thereof. HER3 polypeptide or portions thereof may be expressed on the surface or interior of the vaccine vector. HER3 polynucleotide or portions thereof may be carried within the vaccine vector and the HER3 polypeptide or portions thereof may be expressed only after vaccination. HER3 polypeptides or portions thereof may be expressed as a fusion protein or in conjunction with adjuvants or other immunostimulatory molecules to further enhance the immune response to the polypeptide.

The vaccine vectors may include a promoter operably connected to the polynucleotide encoding any one of the HER3 polypeptides described herein. The vectors may include an origin of replication suitable to allow maintenance of the polynucleotide within a prokaryotic or eukaryotic host cell or within a viral nucleic acid. The vector may be viral vectors including, without limitation, an adenovirus, adeno-associated virus, fowlpox, vaccinia, viral equine encephalitis virus, or venezuelan equine encephalitis virus. In some embodiments, the vector is a DNA-based plasmid vector or DNA vaccine vector.

In some embodiments, the vaccine vector may include an adenovirus serotype 5 vector with E2b, E1, and E3 genes deleted.

The vaccine vector may also be mini-circle DNA (mcDNA) vectors. Mini-circle DNA vectors are episomal DNA vectors that are produced as circular expression cassettes devoid of any bacterial plasmid DNA backbone. See, e.g. System Biosciences, Mountain View Calif., MN501A-1. Their smaller molecular size enables more efficient transfections and offers sustained expression over a period of weeks as compared to standard plasmid vectors that only work for a few days. The minicircle constructs can be derived from a plasmid with a bacterial origin of replication and optionally antibiotic resistance genes flanked by att sites to allow for recombination and exclusion of the DNA between the att sites and formation of the minicircle DNA.

As used herein, a “heterologous promoter” refers to any promoter not naturally associated with a polynucleotide to which it is operably connected. Promoters useful in the practice of the present invention include, without limitation, constitutive, inducible, temporally-regulated, developmentally regulated, chemically regulated, physically regulated (e.g., light regulated or temperature-regulated), tissue-preferred, and tissue-specific promoters. Promoters may include pol I, pol II, or pol III promoters. In mammalian cells, typical promoters include, without limitation, promoters for Rous sarcoma virus (RSV), human immunodeficiency virus (HIV-1), cytomegalovirus (CMV), SV40 virus, and the like as well as the translational elongation factor EF-1α promoter or ubiquitin promoter. Those of skill in the art are familiar with a wide variety of additional promoters for use in various cell types.

Suitably the polynucleotide encodes the full-length HER3 antigenic polypeptide, however, polynucleotides encoding partial, fragment, mutant, variant, or derivative HER3 antigenic polypeptide are also provided. In some embodiments, the polynucleotides may be codon-optimized for expression in a particular cell.

The polynucleotide encoding any of the HER3 polypeptides described herein may also be fused in frame to a second polynucleotide encoding fusion partners such as fusion polynucleotides or polypeptides which provide additional functionality to the antigenic cargo. For example, the second polynucleotide may encode a polypeptide that would target the HER3 polypeptide to the exosome, or would enhance presentation of the HER3 polypeptide, or would stimulate immune responses to the HER3 polypeptide. In some embodiments, the vaccine vectors described herein include a polynucleotide encoding any of the HER3 polypeptides described herein that is fused in frame to a second polynucleotide encoding a lactadherin polypeptide or portions thereof. Lactadherin is a protein that is trafficked to exosomes though its C1C2 domain, a lipid binding domain. The lactadherin polypeptide may include SEQ ID NOS: 35-38 or a homolog thereof.

### Combination Compositions

In another aspect, compositions including any one of the vaccine vectors described herein and a checkpoint inhibitor or a polynucleotide encoding a checkpoint inhibitor are provided.

As used herein, a “checkpoint inhibitor” is an agent, such as antibody or small molecule, which blocks the immune checkpoint pathways in immune cells that are responsible for maintaining self-tolerance and modulating the degree of an immune response. Exemplary checkpoint inhibitors include, without limitation, antibodies or other agents targeting programmed cell death protein 1 (PD1, also known as CD279), programmed cell death 1 ligand 1 (PD-L1, also known as CD274), PD-L2, cytotoxic T-lymphocyte antigen 4 (CTLA4, also known as CD152), A2AR, CD27, CD28, CD40, CD80, CD86, CD122, CD137, OX40, GITR, ICOS, TIM-3, LAG3, B7-H3, B7-H4, BTLA, IDO, KIR, or VISTA. Suitable anti-PD1 antibodies include, without limitation, lambrolizumab (Merck MK-3475), nivolumab (Bristol-Myers Squibb BMS-936558), AMP-224 (Merck), and pidilizumab (CureTech CT-011). Suitable anti-PD-L1 antibodies include, without limitation, MDX-1105 (Medarex), MEDI4736 (Medimmune) MPDL3280A (Genentech/Roche) and BMS-936559 (Bristol-Myers Squibb). Exemplary anti-CTLA4 antibodies include, without limitation, ipilimumab (Bristol-Myers Squibb) and tremelimumab (Pfizer).

In some embodiments, the checkpoint inhibitor may be selected from the group consisting of an anti-PD-1 agent, an anti-PDL1 agent, and an anti-CTLA-4 agent.

In some embodiments, the checkpoint inhibitor may be the form of a polynucleotide encoding a checkpoint inhibitor. For example, with regards to antibody-based checkpoint inhibitors, the checkpoint inhibitor may be in the form of a DNA polynucleotide that is included in any one of the vaccine vectors disclosed herein or may be a DNA polynucleotide that is included in a different expression vector or plasmid. Alternatively, the checkpoint inhibitor may be in the form of a RNA polynucleotide such as, without limitation, an mRNA.

The combination compositions described herein may also include two checkpoint inhibitors, wherein one checkpoint inhibitor comprises an anti-PD-1 agent or an anti-PDL1 agent and the other checkpoint inhibitor comprises an anti-CTLA-4 agent.

The combination compositions may further include a cancer therapeutic or prevention agent. As used herein, a “cancer therapeutic or prevention agent” may be any agent capable of treating the cancer or inhibiting growth of cancer cells. Suitable agents include those which target HER2, HER1/EGFR, estrogen receptor or IGF1R. The cancer therapeutic or prevention agent may be trastuzumab, lapatinib, pertuzumab or another HER2 targeting therapeutic agent or it may be an EGFR targeting therapeutic agent such as cetuximab or erlotanib, or it may be an antiestrogen, or an agent that prevents estrogen synthesis such as an aromatase inhibitor.

### Pharmaceutical Compositions

In a further aspect, pharmaceutical compositions are provided. The pharmaceutical compositions may include a pharmaceutically-acceptable carrier and any one of the vaccine vectors described herein or any one of the combination compositions described herein.

The pharmaceutical compositions may include a pharmaceutical carrier, excipient, or diluent, which are nontoxic to the cell or subject being exposed thereto at the dosages and concentrations employed. Often a pharmaceutical diluent is in an aqueous pH buffered solution. Examples of pharmaceutical carriers include buffers such as phosphate, citrate, and other organic acids; antioxidants including ascorbic acid; low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™ brand surfactant, polyethylene glycol (PEG), and PLURONICS™ surfactant.

The pharmaceutical compositions may include adjuvants to increase immunogenicity of the composition. In some embodiments, these pharmaceutical compositions comprise one or more of a mineral adjuvant, gel-based adjuvant, tensoactive agent, bacterial product, oil emulsion, particulated adjuvant, fusion protein, and lipopeptide. Mineral salt adjuvants include aluminum adjuvants, salts of calcium (e.g. calcium phosphate), iron and zirconium. Gel-based adjuvants include aluminum gel-based adjuvants and acemannan. Tensoactive agents include Quil A, saponin derived from an aqueous extract from the bark of Quillaja saponaria; saponins, tensoactive glycosides containing a hydrophobic nucleus of triterpenoid structure with carbohydrate chains linked to the nucleus, and QS-21. Bacterial products include cell wall peptidoglycan or lipopolysaccharide of Gram-negative bacteria (e.g. from Mycobacterium spp., Corynebacterium parvum, C. granulosum, Bordetella pertussis and Neisseria meningitidis), N-acetyl muramyl-L-alanyl-D-isoglutamine (MDP), different compounds derived from MDP (e.g. threonyl-MDP), lipopolysaccharides (LPS) (e.g. from the cell wall of Gram-negative bacteria), trehalose dimycolate (TDM), cholera toxin or other bacterial toxins, and DNA containing CpG motifs. Oil emulsions include FIA, Montanide, Adjuvant 65, Lipovant, the montanide family of oil-based adjuvants, and various liposomes. Among particulated and polymeric systems, poly (DL-lactide-coglycolide) microspheres have been extensively studied and find use herein. Notably, several of the delivery particles noted above may also act as adjuvants.

In some embodiments, the pharmaceutical compositions further include cytokines (e.g. IFN-γ, granulocyte-macrophage colony stimulating factor (GM-CSF) IL-2, or IL-12) or immunostimulatory molecules such as FasL, CD40 ligand or a toll-like receptor agonist, or carbohydrate adjuvants (e.g. inulin-derived adjuvants, such as, gamma inulin, algammulin, and polysaccharides based on glucose and mannose, such as glucans, dextrans, lentinans, glucomannans and galactomannans). In some embodiments, adjuvant formulations are useful in the present invention and include alum salts in combination with other adjuvants such as Lipid A, algammulin, immunostimulatory complexes (ISCOMS), which are virus like particles of 30-40 nm and dodecahedric structure, composed of Quil A, lipids, and cholesterol.

In some embodiments, the additional adjuvants are described in Jennings et al. Adjuvants and Delivery Systems for Viral Vaccines-Mechanisms and Potential. In: Brown F, Haaheim L R, (eds). Modulation of the Immune Response to Vaccine Antigens. Dev. Biol. Stand, Vol. 92. Basel: Karger 1998; 19-28 and/or Sayers et al. J Biomed Biotechnol. 2012; 2012: 831486, and/or Petrovsky and Aguilar, Immunology and Cell Biology (2004) 82, 488-496.

In some embodiments, the adjuvant is an aluminum gel or salt, such as aluminum hydroxide, aluminum phosphate, and potassium aluminum sulfate, AS04 (which is composed of aluminum salt and MPL), and ALHYDROGEL. In some embodiments, the aluminum gel or salt is a formulation or mixture with any of the additional adjuvants described herein.

In some embodiments, pharmaceutical compositions include oil-in-water emulsion formulations, saponin adjuvants, ovalbumin, Freunds Adjuvant, cytokines, and/or chitosans. Illustrative compositions comprise one or more of the following.

(1) ovalbumin (e.g. ENDOFIT);

(2) oil-in-water emulsion formulations, with or without other specific immunostimulating agents, such as: (a) MF59 (PCT Publ. No. WO 90/14837), which may contain 5% Squalene, 0.5% Tween 80, and 0.5% Span 85 (optionally containing various amounts of MTP-PE) formulated into submicron particles, (b) SAF, containing 10% Squalane, 0.4% Tween 80, 5% pluronic-blocked polymer L121, and thr-MDP either microfluidized into a submicron emulsion or vortexed to generate a larger particle size emulsion, (c) RIBI adjuvant system (RAS), (RIBI IMMUNOCHEM, Hamilton, Mo.) containing 2% Squalene, 0.2% Tween 80, and, optionally, one or more bacterial cell wall components from the group of monophosphorylipid A (MPL), trehalose dimycolate (TDM), and cell wall skeleton (CWS), including MPL+ CWS (DETOX™); and (d) ADDAVAX (Invitrogen);

(3) saponin adjuvants, such as STIMULON (Cambridge Bioscience, Worcester, Mass.);

(4) Complete Freunds Adjuvant (CFA) and Incomplete Freunds Adjuvant (IFA);

(5) cytokines, such as interleukins (by way of non-limiting example, IL-1, IL-2, IL-4, IL-5, IL-6, IL-7, IL-12, etc.), interferons (e.g., gamma interferon), macrophage colony stimulating factor (M-CSF), tumor necrosis factor (TNF), etc;

(6) chitosans and other derivatives of chitin or poly-N-acetyl-D-glucosamine in which the greater proportion of the N-acetyl groups have been removed through hydrolysis; and

(7) other substances that act as immunostimulating agents to enhance the effectiveness of the composition, e.g., monophosphoryl lipid A.

In other embodiments, adjuvants include a flagellin-based agent, an aluminium salt or gel, a pattern recognition receptors (PRR) agonist, CpG ODNs and imidazoquinolines. In some embodiments, adjuvants include a TLR agonist (e.g. TLR1, and/or TLR2, and/or TLR3, and/or TLR4, and/or TLR5, and/or TLR6, and/or TLR7, and/or TLR8, and/or TLR9, and/or TLR10, and/or TLR11, and/or TLR12, and/or TLR13), a nucleotide-binding oligomerization domain (NOD) agonist, a stimulator of interferon genes (STING) ligand, or related agent.

### Methods of Treatment

Methods of treating a cancer or precancer, or of reducing the likelihood of the cancer or precancer developing resistance to a cancer therapeutic or prevention agent, are also provided. The methods include administering the vaccine as described above to a subject having cancer or precancer. The subject may be any mammal, suitably a human, domesticated animal such as a dog or cat, or a mouse or rat. A cancer therapeutic or prevention agent may be administered concurrently with, before or after administration of the vaccine.

The cancer therapeutic or prevention agents may be any agent capable of treating the cancer or inhibiting growth of cancer cells or increasing the immune response to the cancer. Suitable agents include those which target HER2, HER1/EGFR, estrogen receptor or IGF1R. The therapeutic agent may be trastuzumab, lapatinib, pertuzumab or another HER2 targeting therapeutic agent or it may be an EGFR targeting therapeutic agent such as cetuximab or erlotanib, or it may be an antiestrogen, or an agent that prevents estrogen synthesis such as an aromatase inhibitor. In particular, the Examples demonstrate that a HER3 vaccine can treat a HER2 positive cancer when used in combination with a therapeutic agent targeting HER2. Cancer cells often develop resistance to HER2 targeting therapeutic agents. Addition of vaccination with a HER3 vaccine or passively transferred polyclonal antibodies specific for HER3 resulted in blocking resistance, inhibited cancer cell growth and resulted in treatment of the cancer.

In a still further aspect, methods of treating a cancer or precancer, or of reducing the likelihood of the cancer or precancer developing resistance to a cancer therapeutic or prevention agent in a subject are provided. The methods may include administering a therapeutically effective amount of any one of the combination compositions described herein to the subject having the cancer or precancer. Alternatively, the methods may include administering a therapeutically effective amount of any one of the vaccine vectors described herein to the subject having the cancer or precancer, and administering a therapeutically effective amount of a checkpoint inhibitor or a polynucleotide encoding a checkpoint inhibitor. Optionally, each of these methods may further include administering a therapeutically effective amount of the cancer therapeutic or prevention agent to the subject.

In some embodiments of the present methods, two checkpoint inhibitors may be administered wherein one checkpoint inhibitor comprises an anti-PD-1 agent or an anti-PDL1 agent and the other checkpoint inhibitor comprises an anti-CTLA-4 agent.

In some embodiments, the administration of the vaccine vector and the checkpoint inhibitor results in decreased tumor growth rate or decreased tumor size after administration as compared to administration of either the vaccine vector or checkpoint inhibitor alone.

Suitably the vaccinated subject develops an immune response to HER3 in response to administration of the vaccine. The immune response may be an antibody or T cell immune response. For example the immune response may include antibody-dependent cellular cytotoxicity, polyclonal antibody response, complement dependent cellular cytotoxicity, cellular cytotoxicity, disruption of ligand binding, disruption of dimerization, mimicking ligand binding causing internalization of HER3, or degradation of HER3. The immune response may comprise an antibody response directed to at least one of SEQ ID NOs: 5-22. As shown in the Examples, transfer of HER3 specific antibodies was sufficient to treat the cancer and inhibit the development of resistance to the therapeutic agent.

Reduction of the development of resistance can be measured in several ways. The resistance of the vaccinated subject may be compared to a similar subject that was not vaccinated as in the Examples. Alternatively, the reduction may be measured based on statistics generated regarding the likelihood of an individual being treated with the therapeutic agent to develop resistance versus that of individuals treated with the therapeutic agent and vaccinated with HER3. The reduction in the likelihood of resistance of the cancer may also be measured by measuring the level of HER3 expression on the surface of cancer cells. HER3 expression is reduced on cancer cells after effective administration of the vaccine. The effectiveness of the vaccine in treating the cancer or reducing the likelihood of resistance can be measured by tracking the growth of the tumor or the growth rate of the tumor or cancer cells. A decrease in tumor size or in the rate of tumor growth is indicative of treatment of the cancer.

The cancer may be selected from any cancer capable of developing resistance to a therapeutic agent by increasing expression or activation of a protein by the cancer cells. In particular the cancer may be any cancer capable of developing resistance to a therapeutic agent which targets a HER family tyrosine kinase, suitably HER2 or EGFR or the estrogen receptor, suitably anti-estrogens. The cancer may develop resistance by increasing the expression of HER3, which although not a kinase, will dimerize with another HER family kinase and allow for signaling to occur.

Exemplary cancers in accordance with the present invention include, without limitation, primary and metastatic breast, ovarian, liver, pancreatic, prostate, bladder, lung, osteosarcoma, pancreatic, gastric, esophageal, colon, skin cancers (basal and squamous carcinoma; melanoma), testicular, colorectal, urothelial, renal cell, hepatocellular, leukemia, lymphoma, multiple myeloma, head and neck, and central nervous system cancers or pre-cancers. In some embodiments, the cancer may be HER2 positive. The cancer may be selected from any cancer capable of developing resistance to a therapeutic agent by increasing expression or activation of a protein by the cancer cells. In particular the cancer may be any cancer capable of developing resistance to a therapeutic agent which targets a HER family tyrosine kinase, suitably HER2 or EGFR or the estrogen receptor, suitably anti-estrogens. The cancer may develop resistance by increasing the expression of HER3, which although not a kinase, will dimerize with another HER family kinase and allow for signaling to occur.

The resistance may be due to a single or multiple changes, and the vaccine can target one or more of these changes, and/or include multiple antigens likely found in resistance cells, but not necessarily in all resistance cells. Thus the HER3 vaccine vectors provided herein may be administered in combination with other therapeutic agents including those targeting a HER family kinase such a s HER2 or EGFR such as a tyrosine kinase inhibitor or may be combined with a checkpoint inhibitor or may be combined with both a HER targeting agent and a checkpoint inhibitor. The vaccines need not be administered at the same time as the other agents. The HER3 vaccine vectors may be administered before, at the same time or after the other agents. In addition to the HER3 vaccine vectors provided herein, other vaccine vectors may also be used such as those in published applications WO 2016/007499; WO 2016/007504; and WO 2017/120576. Each of these vaccine vectors may be combined with at least one checkpoint inhibitor.

Treating cancer includes, but is not limited to, reducing the number of cancer cells or the size of a tumor in the subject, reducing progression of a cancer to a more aggressive form (i.e. maintaining the cancer in a form that is susceptible to a therapeutic agent), reducing proliferation of cancer cells or reducing the speed of tumor growth, killing of cancer cells, reducing metastasis of cancer cells or reducing the likelihood of recurrence of a cancer in a subject. Treating a subject as used herein refers to any type of treatment that imparts a benefit to a subject afflicted with cancer or at risk of developing cancer or facing a cancer recurrence. Treatment includes improvement in the condition of the subject (e.g., in one or more symptoms), delay in the progression of the disease, delay in the onset of symptoms or slowing the progression of symptoms, etc.

Co-administration, or administration of more than one composition (i.e. a vaccine and a therapeutic agent) to a subject, indicates that the compositions may be administered in any order, at the same time or as part of a unitary composition. The two compositions may be administered such that one is administered before the other with a difference in administration time of 1 hour, 2 hours, 4 hours, 8 hours, 12 hours, 16 hours, 20 hours, 1 day, 2 days, 4 days, 7 days, 2 weeks, 4 weeks or more.

In some embodiments, the vaccine vector is administered prior to or simultaneously with the checkpoint inhibitor.

In some embodiments, the vaccine vector is administered prior to the administration of the optional cancer therapeutic or prevention agent.

An effective amount or a therapeutically effective amount as used herein means the amount of a composition that, when administered to a subject for treating a state, disorder or condition is sufficient to effect a treatment (as defined above). The therapeutically effective amount will vary depending on the compound, formulation or composition, the disease and its severity and the age, weight, physical condition and responsiveness of the subject to be treated.

The compositions (i.e. the vaccines and the therapeutic agents) described herein may be administered by any means known to those skilled in the art, including, but not limited to, oral, topical, intranasal, intraperitoneal, parenteral, intravenous, intramuscular, subcutaneous, intrathecal, transcutaneous, nasopharyngeal, or transmucosal absorption. Thus the compositions may be formulated as an ingestable, injectable, topical or suppository formulation. The compositions may also be delivered with in a liposomal or time-release vehicle. Administration of the compositions to a subject in accordance with the invention appears to exhibit beneficial effects in a dose-dependent manner. Thus, within broad limits, administration of larger quantities of the compositions is expected to achieve increased beneficial biological effects than administration of a smaller amount. Moreover, efficacy is also contemplated at dosages below the level at which toxicity is seen.

It will be appreciated that the specific dosage administered in any given case will be adjusted in accordance with the composition or compositions being administered, the disease to be treated or inhibited, the condition of the subject, and other relevant medical factors that may modify the activity of the compositions or the response of the subject, as is well known by those skilled in the art. For example, the specific dose for a particular subject depends on age, body weight, general state of health, diet, the timing and mode of administration, the rate of excretion, medicaments used in combination and the severity of the particular disorder to which the therapy is applied. Dosages for a given patient can be determined using conventional considerations, e.g., by customary comparison of the differential activities of the compositions described herein and of a known agent, such as by means of an appropriate conventional pharmacological or prophylactic protocol.

The maximal dosage for a subject is the highest dosage that does not cause undesirable or intolerable side effects. The number of variables in regard to an individual prophylactic or treatment regimen is large, and a considerable range of doses is expected. The route of administration will also impact the dosage requirements. It is anticipated that dosages of the compositions will reduce the growth of the cancer at least 10%, 20%, 30%, 40%, 50%, 60%, 70%, 80%, 90%, 100% or more as compared to no treatment or treatment with only the therapeutic agent. It is specifically contemplated that pharmaceutical preparations and compositions may palliate, block further growth or alleviate symptoms associated with the cancer without providing a cure, or, in some embodiments, may be used to cure the cancer and rid the subject of the disease.

The effective dosage amounts described herein refer to total amounts administered, that is, if more than one composition is administered, the effective dosage amounts correspond to the total amount administered. The compositions can be administered as a single dose or as divided doses. For example, the composition may be administered two or more times separated by 4 hours, 6 hours, 8 hours, 12 hours, a day, two days, three days, four days, one week, two weeks, or by three or more weeks.

The vaccine vector may be administered one time or more than one time to the subject to effectively boost the immune response against HER3. If the vaccine is provided as a vaccine vector, the vaccine vector may be administered based on the number of particles delivered to the subject (i.e. plaque forming units or colony forming units). The subject may be administered 10¹², 10¹¹, 10¹⁰, 10⁹, 10⁸, 10⁷ or 10⁶ particles.

The present disclosure is not limited to the specific details of construction, arrangement of components, or method steps set forth herein. The compositions and methods disclosed herein are capable of being made, practiced, used, carried out and/or formed in various ways that will be apparent to one of skill in the art in light of the disclosure that follows. The phraseology and terminology used herein is for the purpose of description only and should not be regarded as limiting to the scope of the claims. Ordinal indicators, such as first, second, and third, as used in the description and the claims to refer to various structures or method steps, are not meant to be construed to indicate any specific structures or steps, or any particular order or configuration to such structures or steps. All methods described herein can be performed in any suitable order unless otherwise indicated herein or otherwise clearly contradicted by context. The use of any and all examples, or exemplary language (e.g., “such as”) provided herein, is intended merely to facilitate the disclosure and does not imply any limitation on the scope of the disclosure unless otherwise claimed. No language in the specification, and no structures shown in the drawings, should be construed as indicating that any non-claimed element is essential to the practice of the disclosed subject matter. The use herein of the terms “including,” “comprising,” or “having,” and variations thereof, is meant to encompass the elements listed thereafter and equivalents thereof, as well as additional elements. Embodiments recited as “including,” “comprising,” or “having” certain elements are also contemplated as “consisting essentially of” and “consisting of” those certain elements.

Recitation of ranges of values herein are merely intended to serve as a shorthand method of referring individually to each separate value falling within the range, unless otherwise indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure. Use of the word “about” to describe a particular recited amount or range of amounts is meant to indicate that values very near to the recited amount are included in that amount, such as values that could or naturally would be accounted for due to manufacturing tolerances, instrument and human error in forming measurements, and the like. All percentages referring to amounts are by weight unless indicated otherwise.

No admission is made that any reference, including any non-patent or patent document cited in this specification, constitutes prior art. In particular, it will be understood that, unless otherwise stated, reference to any document herein does not constitute an admission that any of these documents forms part of the common general knowledge in the art in the United States or in any other country. Any discussion of the references states what their authors assert, and the applicant reserves the right to challenge the accuracy and pertinence of any of the documents cited herein. All references cited herein are fully incorporated by reference, unless explicitly indicated otherwise. The present disclosure shall control in the event there are any disparities between any definitions and/or description found in the cited references. The following examples are meant only to be illustrative and are not meant as limitations on the scope of the invention or of the appended claims.

### EXAMPLES

### Example 1

### Materials and Methods

### Cell Lines and Cell Culture Reagents.

The human breast cancer cell lines BT474, MCF-7, MDA-MB-231, MDA-MB-468, SKBR3. and T47D were obtained from the ATCC and grown in recommended media. The BT474M1 human breast tumor cell line was a gift from Dr. Mien-Chie Hung at The University of Texas M. D. Anderson Cancer Center and was grown in DMEM/F12 with 10% FBS. Laptinib-resistant BT474 (rBT474) were generated as previously described. Xia et al. A model of acquired autoresistance to a potent ErbB2 tyrosine kinase inhibitor and a therapeutic strategy to prevent its onset in breast cancer. Proc Natl Acad Sci U S A 2006;103:7795-800. Trastuzumab (Herceptin™, Genentech, San Francisco, Calif.) was purchased from the Duke Pharmacy.

### Adenovirus Vector Preparation.

The human HER3 cDNA was excised from a pCMVSport6-HER3-HsIMAGE6147464 plasmid (cDNA clone MGC:88033/IMAGE:6147464) from the ATCC (Manassas, Va.), and construction of first-generation [E1-, E3-] Ad vectors containing human full length HER3 under control of human CMV promoter/enhancer elements was performed using the pAdEasy system (Agilent technologies, Santa Clara, Calif.) as previously described. Morse et al. Synergism from combined immunologic and pharmacologic inhibition of HER2 in vivo. Int J Cancer 2010;126:2893-903; Amalfitano et al. Production and characterization of improved adenovirus vectors with the E1, E2b, and E3 genes deleted. J Virol 1998;72:926-33; Hartman et al. An adenoviral vaccine encoding full-length inactivated human Her2 exhibits potent immunogenicty and enhanced therapeutic efficacy without oncogenicity. Clin Cancer Res 2010;16:1466-77; and He et al. A simplified system for generating recombinant adenoviruses. Proc Natl Acad Sci U S A 1998;95;2509-14.

### Mice.

BALB/c and NOD.CB17-Prkdc^{scid}/J mice were purchased from Jackson Labs (Bar Harbor, Me.). All work was conducted in accordance with Duke IACUC-approved protocols. Induction of VIA: BALB/c mice were vaccinated on day 0 and day 14 via.

footpad injection with Ad-GFP, or Ad-HER3 vectors (2.6×10¹⁰ particles/mouse). Fourteen days after the second vaccination, mice were euthanized and sera were collected and stored at −80° C.

### MTT Assay to Detect Cell Proliferation.

The effect of VIA-HER3 on the proliferation of human breast cancer cell lines was measured as previously described. Morse et al. Synergism from combined immunologic and pharmacologic inhibition of HER2 in vivo. Int J Cancer 2010;126:2893-903. Briefly, 5000 cells per well in a 96-well plate were cultured with HER3-VIA. (1:33 dilution) or control serum GFP-VIA (1:33 dilution) or Trastuzumab 20 μg/ml for 3 days and proliferation was assessed by MTT assay.

### Western Blotting to Analyze Pathway Inhibition.

Tumors were isolated from euthanized mice and immediately flash frozen. Tissue extracts were prepared by homogenization in RIPA buffer as previously described by Morse et al. 2010. Equal amounts of proteins (50 ug) were resolved by 4-15% gradient SDS PAGE After transfer membranes were probed with specific antibodies recognizing target proteins: pTyr (Sigma), ErbB2, ErbB3, Akt, pAkt473, Erk 1/2, pErk1/2, (Cell Signaling, Beverly, Mass.) survivin, and actin (Sigma, St. Louis, Mo.), 4EBP-1, p4EBP-1, s6, ps6 (Santa Cruz Biotech., Santa Cruz, Calif.) and IRDye 800 conjugated anti-rabbit or mouse IgG or Alexa Fluor 680 anti-rabbit IgG and were visualized using the Odyssey Infrared Imaging System (LI-COR, Lincoln, Nebr.).

### ELISPOT Analysis.

IFN-gamma ELISPOT assays (Mabtech, Cincinnati, Ohio) performed as previously described by Morse et al, 2010. HER3 peptide mix (1 mcg/mL was used; Jerini Peptide Technologies, Berlin, Germany), HIVgag peptide mix (BD Bioscience), or a mixture of PMA (50 ng/mL) and Ionomycin (1 ng/mL) were used. Six replicate wells for each condition were scored using the KS ELISPOT Reader with the KS ELISPOT 4.9 Software (Carl Zeiss, München-Hallbergmoos, Germany), reporting responses as the mean of the replicate 6 wells.

### Analysis of Anti-HERS Antibody Binding by Flow Cytometry.

We have adapted a methodology reported by Piechocki et al. to measure anti-HER3 vaccine induced antibodies in vaccinated mouse serum by flow cytometry. Hartman et al. An adenoviral vaccine encoding full-length inactivated human Her2 exhibits potent immunogenicty and enhanced therapeutic efficacy without oncogenicity.

Clin Cancer Res 2010;16:1466-77 and Piechockiet al. Quantitative measurement of anti-ErbB-2 antibody by flow cytometry and ELISA. J Immunol Methods 2002;259:33-42. Briefly, 3×10⁵ human breast cancer cells were incubated with diluted (1:100 to 1:51,200) mouse serum antibodies (HER3-VIA or GFP-VIA) for 1 h at 4° C. and then washed with 1% BSA-PBS. The cells were further stained with PE-conjugated anti-mouse IgG (Dako, Cat #R0480) for 30 minutes at 4° C., and washed again. Samples were analyzed on a BD LSRII flow cytometer (Becton Dickenson, San Jose, Calif.) and mean fluorescence intensity (MFI) reported.

### Complement Dependent Cytotoxicity Assay.

We performed complement dependent cytotoxicity assays using our previously published protocol in Morse et al. 2010. Briefly, target cells were incubated with rabbit serum (1:100) as a source of complement and the HER3-VIA or GFP-VIA in sera from mice immunized as above diluted (1:100), or Trastuzumab (20 mcg/ml) at 37° C. for 2 hrs. After incubation, cytotoxicity was measured using the CytoTox 96 Nonradioactive Cytotoxicity Assay (Promega; per manufacturer's instructions) to measure LDH release in the culture media as evidence of cytotoxicity.

### Assessment of HER3 Internalization

Human HER3+ breast cancer cells (SKBR3 and BT474M1) were incubated with 1:100 HER3-VIA or GFP-VIA at 37° C. for 60 minutes. After washing, fixation with 4% PFA, and permeabilization with permeabilizing solution 2 (Becton Dickenson), nonspecific binding was blocked with 2.5% Goat Serum at 37° C. for 30 min. Cells were incubated with 1:100 Red™-conjugated anti-mouse IgG (H+L) (Jackson ImmunoResearch Laboratories Inc. West Grove, Pa.) in a dark chamber for 1 hour at room temperature and washed with PBS. Slides were mounted in VectaShield containing DAPI (Vector Laboratories, Burlingame, Calif.) and images acquired using a Zeiss Axio Observer widefield fluorescence microscope (Carl Zeiss, Munchen-Hallbergmoos, Germany).

### Treatment of Established HER3+ BT474M1 Human Tumor Xenografts by Passive Transfer of Vaccine Induced Antibodies.

Eight to 10 week old NOD.CB17-Prkdc^{scid}/J mice (Jackson Labs., Bar Harbor, Me.) were implanted in the back with 17 Beta-Estradiol pellets (0.72 mg 60 day continuous release pellets; Innovative Research of American, Sarasota, Fla.) two days prior to tumor implantation. Five million BT474M1 tumor cells in 50% Matrigel were injected into the mammary fat pad. Tumors were allowed to develop for 14 days and then mice were randomized to receive iv injection of either GFP-VIA or HER3-VIA (5 mice per group). 100-150 microliters of VIA was injected at 2-3 day intervals for a total of 10 administrations. Tumor growth was measured in two dimensions using calipers and tumor volume determined using the formula volume=½[(width)×(length)].

Treatment of established HER3+ lapatinib-resistant rBT474 human tumor xenografts by passive transfer of vaccine induced antibodies.

Eight to 10 week old NOD.CB17-Prkdc^{scid}/J mice (Jackson Labs., Bar Harbor, Me.) were implanted in the mammary fat pad with 1 million lapatinib-resistant rBT474 tumor cells in 50% Matrigel. Tumors were allowed to develop for two months and then mice were randomized to receive iv injection of either GFP-VIA or HER2-VIA (5 mice per group). 100-150 microliters of VIA was injected at 2-3 day intervals for a total of 10 administrations. Tumor growth was measured as described above.

### Statistical Analyses.

Tumor volume measurements for in vivo models were analyzed under a cubic root transformation to stabilize the variance as in Morse et al. 2010. Welch t-tests were used to assess differences between mice injected with HER3-VIA or control GFP-VIA. Analyses were performed using R version 2.10.1. For all tests, statistical significance was set at p<0.05.

### Results

### Ad-HER3 Elicits Anti-HER3 T Cell and Antibody Responses In Vivo

We developed a recombinant E1-, E3- adenovirus serotype 5 vector (Ad-HER3) expressing full length human HER3 (Ad-HER3). Wild type BALB/c mice were vaccinated with Ad-HER3, splenocytes from vaccinated mice were harvested and demonstrated by ELISPOT to specifically recognize HER3 using an overlapping human HER3 peptide mix as a source of antigens, whereas splenocytes from mice receiving control Ad-GFP vaccine or saline showed no reactivity to the HER3 peptide mix (FIG. 1A). To detect HER3-specific antibodies capable of detecting membrane associated HER3, binding of vaccine induced antibodies (VIA) in mouse serum was tested using a series of human HER3 expressing breast tumor cells lines, including the high HER3 expressing BT474M1, BT474, SKBR3 and T47D and the low to negatively expressing MDA-231 tumor cell line (FIGS. 1B and 1C). The serum of mice vaccinated with the Ad-HERS had binding titers of >1:800, whereas the serum of mice receiving the control Ad-LacZ vaccine showed only background levels of binding. Thus, HERS-VIA are able to bind to endogenous HER3 expressed on human breast cancer lines.

To confirm that multiple HER3 epitopes were recognized, we demonstrated VIA binding to a series of HER3 peptides. The HER3-VIA recognized at least 18 epitopes in both the intracellular and extracellular domain, demonstrating that the antibody responses are polyclonal (FIG. 1D and SEQ ID NOs: 5-22), It should be noted that peptide arrays do not recapitulate conformationally correct protein structure, so they often underestimate the true number of epitopes recognized.

### HER3 Specific Antibodies Induced by Vaccination (HER3-VIA) Mediate Complement Dependent Lysis of HER3+ Breast Tumor Cell Lines In Vitro

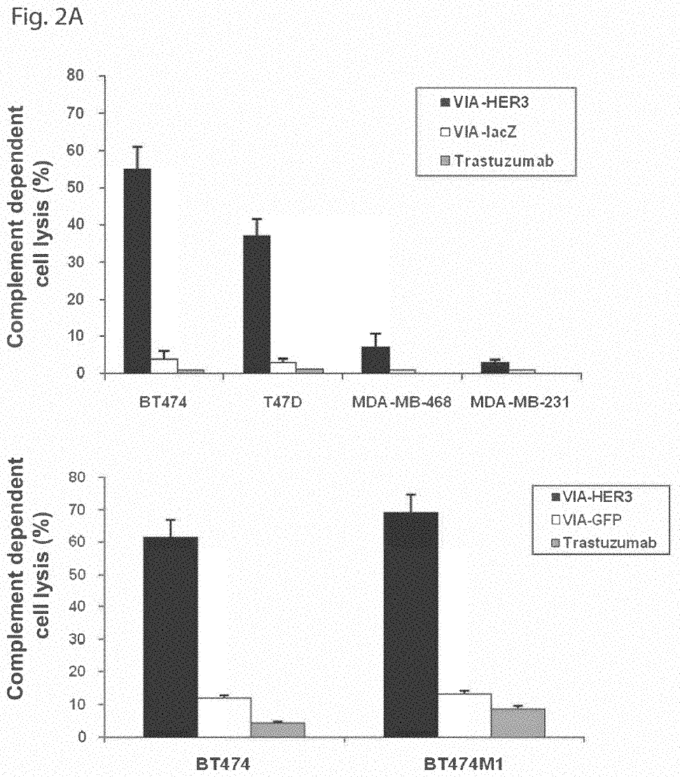

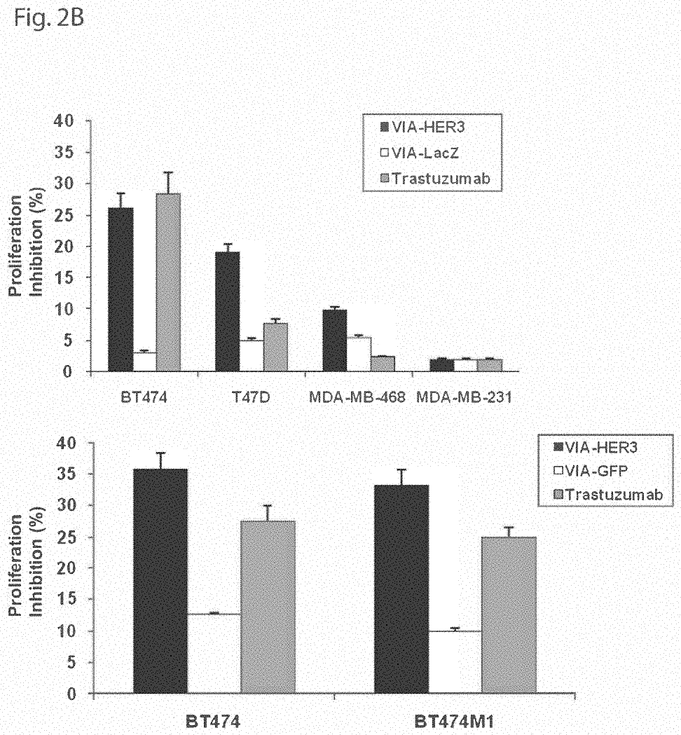

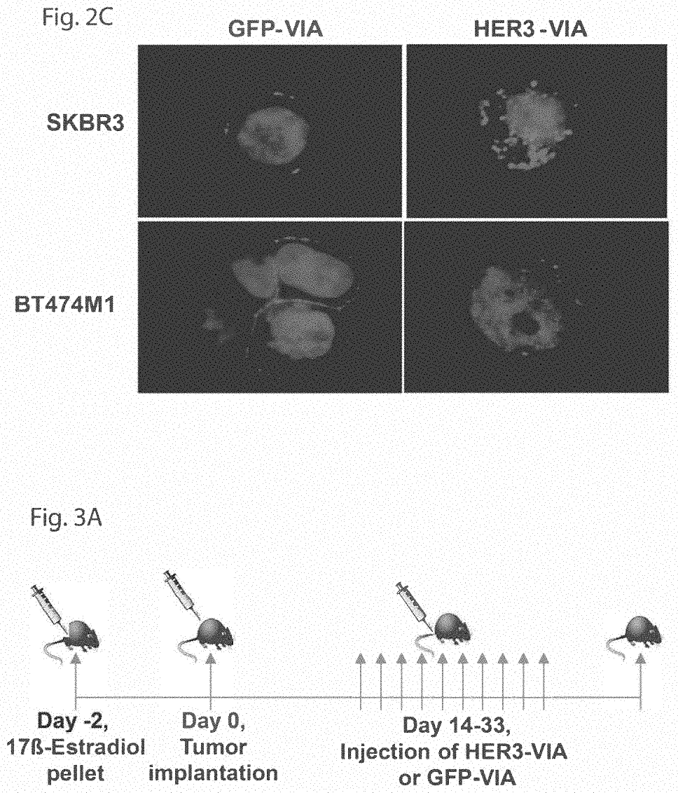

Direct antibody-mediated tumor cell killing is a powerful potential mechanism of action of antibodies induced by vaccination. We evaluated the capacity HER3-VIA to mediate complement-dependent cytotoxicity (CDC). HERS-VIA exhibited strong CDC against HERS-expressing human breast tumor cells but not the HERS negative MDA-231 cell line, while control CEP-VIA showed no effect (FIG. 2A). Trastuzumab is known not to mediate CDC and this was confirmed in our assays.

### Anti-Proliferative Effects of HER3 VIA In Vitro

Although immunization with Ad-HER3 was able to efficiently induce humoral immunity in vivo and mediate complement dependent tumor cell cytotoxicity, we also wished to determine whether these antibodies could inhibit tumor cell proliferation, We found that when HER3-expressing human breast cancer cells were cultured with HER3-VIA from the sera of Ad-HER3 vaccinated mice, their proliferation was significantly inhibited compared with cells cultured with control GFT-VIA (FIG. 2B). Of interest, despite the much high levels of HER2 expressed on these tumor cells, compared to HERS, the inhibition of tumor cell proliferation mediated by HER3-VIA was similar to the effects of the clinically effective monoclonal antibody trastuzumab.

### Loss of HER3 Expression on Tumor Cell Lines Mediated by HER3-VIA In Vitro

Growth factor receptor internalization, degradation, and down regulation has been proposed as a mechanism for the inhibition of tumor growth mediated by monoclonal antibodies. To ascertain whether receptor down regulation was caused by HER3-VIA as a result of receptor internalization, we visualized cell membrane associated HER3 receptor on SKBR3 and BT474M1 tumor cells. When exposed to serum containing HER3-VIA or GFP-VIA, dramatic internalization and aggregation of the receptor was observed within 1 hr after exposure to HER3-VIA, but not with exposure to control GFP-VIA (FIG. 2C).

Inhibition of Tumor Growth by HER3 VIA In Vivo is Associated with Loss of HER3 Expression and Anti-Signaling Effects

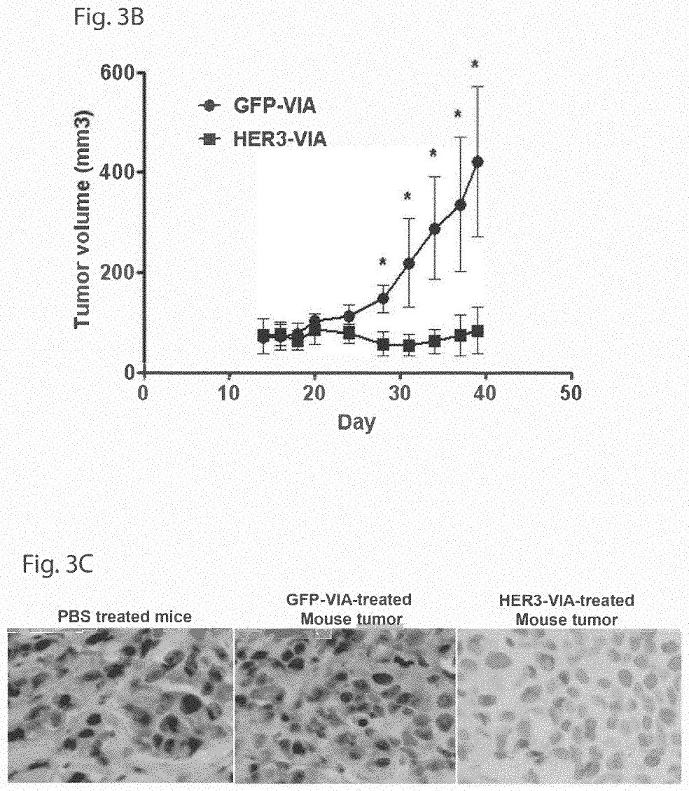

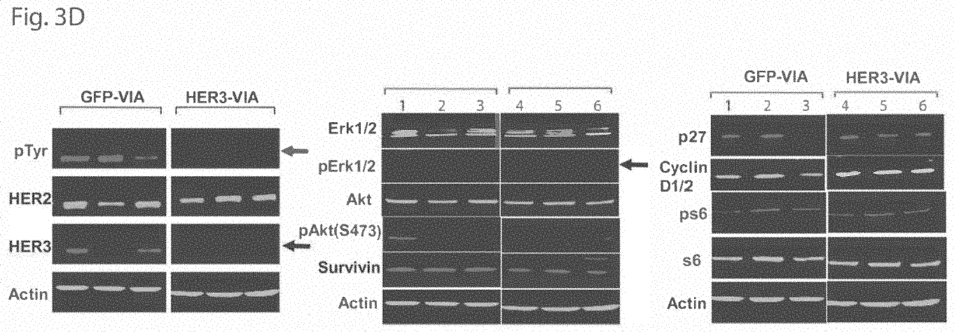

After finding that HER3 specific antibodies could inhibit HER3+ tumor cell proliferation in vitro, we sought to demonstrate the effects of HER3 VIA in vivo. At this time, there are no murine breast tumors dependent on human HER3 for growth, and attempts to establish 4T1 tumors expressing HER3 have been unsuccessful. Consequently, we employed a human xenograft model using the BT474M1 cell line that expresses both HER2 and HER3, with adoptive transfer of antibodies to demonstrate the in vivo activity of HER3-VIA. The study design is illustrated in FIG. 3A. We found that passive immunotherapy with HER3-VIA retarded the growth of established HER3+ BT474M1 human tumor xenografts in vivo (p<0.005 after Day 28) when compared to the control GFP-VIA treated mice (FIG. 3B). At the termination of the study tumor size was compared and was significantly reduced in the HER3-VIA-treated mice (p=0.005).

In addition to demonstrating anti-tumor effects in vivo, we also wanted to document the anti-HER3 signaling effects of HER3 VIA in vivo. Analysis of excised tumors allowed us to determine HER3 expression following treatment in vivo. We found that mice treated with HER3-VIA showed decreased levels of HER3 in their residual tumor by immunohistochemistry (FIG. 3C), consistent with antigen downregulation as the basis of immunologic escape. We also examined the impact of treatment with HER3-VIA on downstream effectors of HER3 signaling, and found a reduction of pHER (pTyr), HER3, and pErk1/2, compared to tumors treated with GFP-VIA (FIG. 3D).

### Inhibition of Therapy-Resistant Tumor Growth by HER3 VIA In Vivo

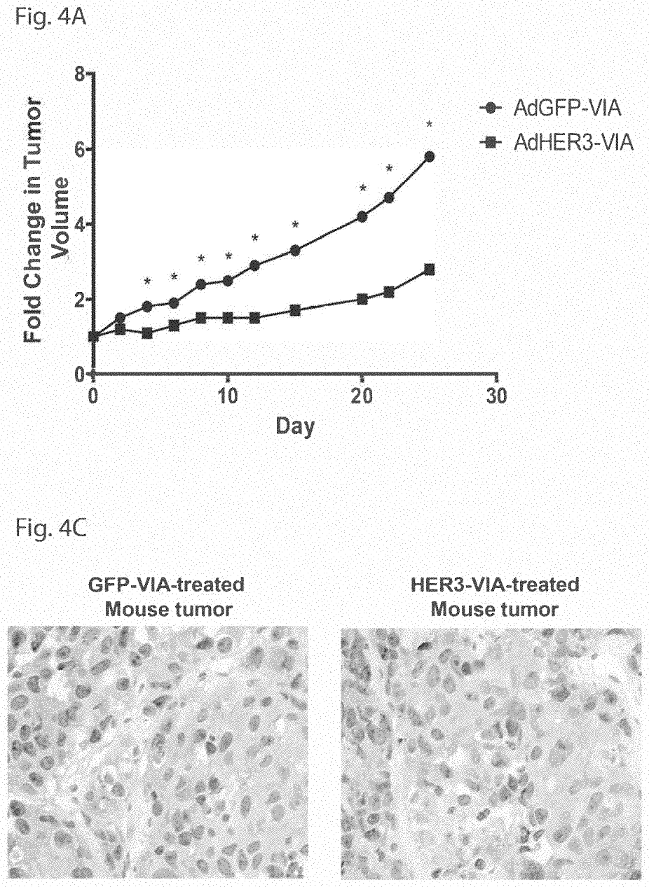

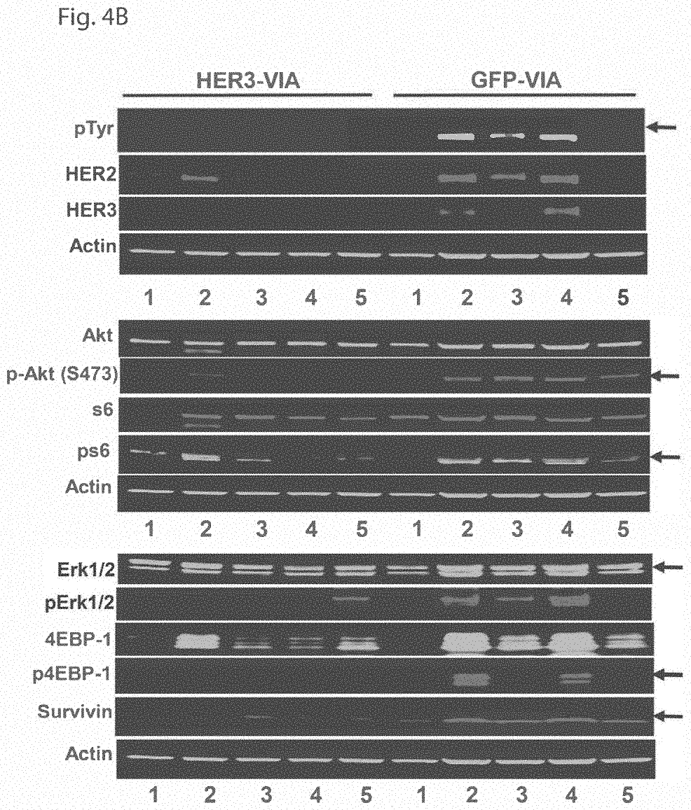

While the antitumor efficacy against established HER3+ BT474M1 tumors was encouraging, we know that a major unmet need for breast cancer patients is for therapies to overcome therapeutic resistance to HER2 targeted therapies. For example, therapeutic resistance to trastuzumab, can be overcome by treatment with a small molecule inhibitor of HER2, lapatinib, but patients whose tumors initially respond ultimately experience therapeutic resistance and disease progression. Of interest is the persistent overexpression of HER2 in the tumors from these patients, and the emerging recognition that signaling from the HER2/HER3 heterodimer, and other heterodimers involving HER3, was a significant resistance mechanism. Consequently, we tested the effects of HER3-VIA in a model of lapatinib resistance derived from the rBT474 cell line that we have previously reported. Xia et al. A model of acquired autoresistance to a potent ErbB2 tyrosine kinase inhibitor and a therapeutic strategy to prevent its onset in breast cancer. Proc Natl Acad Sci USA 2006;103:7795-800. This rBT474 cell line expresses HER2 and HER3 at similar levels to the BT474M1 tumor line. We demonstrate that the HER3-VIA was effective at retarding the growth of established tumors (FIG. 4) (p 0.025 for all time points from Day 4 to Day 25), confirming the therapeutic potential of an Ad-HER3 vaccine for patients who have experienced disease progression on lapatinib.

Inhibition of Lapatinib-Resistant Tumor Growth by HER3 VIA In Vivo is Associated with Loss of HER3 Expression and Broader Anti-Signaling Effects than Lapatinib-Sensitive Tumors

Tumors excised from the mice at the termination of the study described above, were examined for signaling pathway modulation. Whole tumor lysates from 5 mice per group were studied, since we expected some mouse-to-mouse variation and wanted to capture the spectrum of responses (FIG. 4B). Total HER2 and HER3 levels are decreased in the HER3-VIA treated tumors, suggesting receptor degradation may be occurring. pTyr is also consequently reduced, indicating decreased HER2:HER3 signaling. pAkt473(5473) and pS6 are also decreased for the HER3-VIA treated tumors, as are pErk1/2, p4EBP1, and survivin relative to the control GFP-VIA treated tumors. In contrast to the data in the lapatinib-sensitive BT474M1 tumors, immunohistochemistry analysis of excised rBT474 tumors did not show a marked decrease in HER3 in tumors treated with HER3-VIA compared to GFP-VIA controls (FIG. 4C), suggesting that HER3 degradation was more modest and anti-proliferative effects mediated through the HER3 heterodimers were therefore more prominent.

### Generate Ad5(E2b-)HER3 and Ad5(E2b-)HER3 C1C2 Constructs (Y1, Q1-2)

Adenoviral vectors expressing HER3 using the Ad5(E2b-) platform have been constructed and have been used to generate virus. We now wanted to assess whether other HER3 expressing adenovirus vectors would have similar effects. We have modified the adenovirus construction methods to facilitate the construction of (E1-, E2b-, E3-) Ad5 vector.

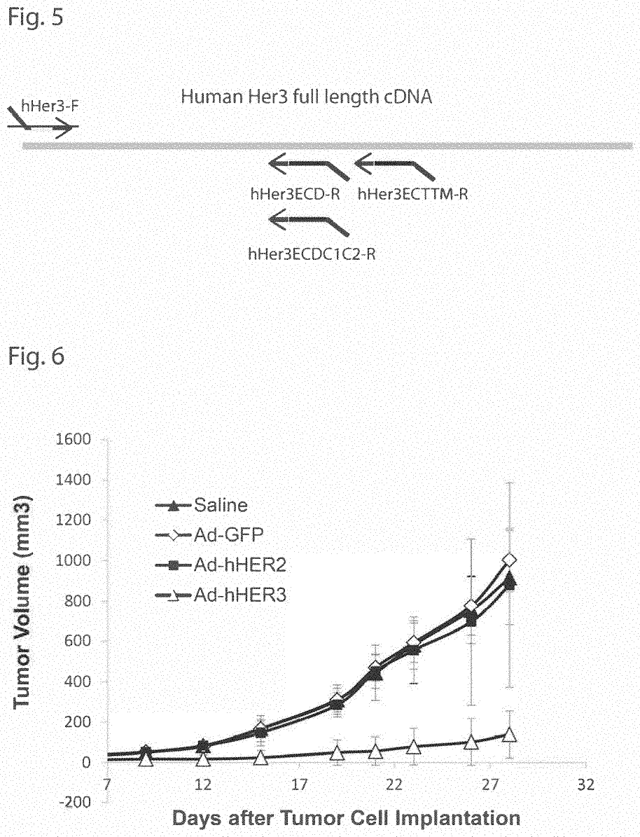

The human HER3 full length cDNA was obtained from OriGene (Rockville, Md.). The truncated HER3 extracellular domain (ECD) and HER3 ECD plus transmembrane (TM) sequence were created using HER3 full length as templates in a PCR reaction using primers (see Table 1 below) and FIG. 5.

TABLE 1
Primers used in construction of truncated
Ad5-human HER3

| Primer | Sequence (SEQ ID NO:) |
|---|---|
| hHER3-F | 5′-cagggcggccgcaccatgagggcgaacgac |
| | gctct-3′ (SEQ ID NO: 23) |
| hHER3-ECDTM-R | 5′-acaagcggccgcagttaaaaagtgccgccc |
| | agcatca-3′ (SEQ ID NO: 24) |
| hHER3-ECD-R | 5′-acaagcggccgcatttatgtcagatgggtt |
| | ttgccgatc-3′ (SEQ ID NO: 25) |
| hHER3-ECDC1C2-R | 5′-acaagcggccgcattgtcagatgggttttg |
| | ccg-3′ (SEQ ID NO: 26) |

Briefly, full length HER3 cDNA and the PCR product are cut by restrict enzyme Not I and subcloned into Not I digested pShuttle-CMV or pShuttleCMV-C1C2 plasmid. Confirmation of correct insert of the full length and truncated DNA within pShuttle-CMV or pShuttle-CMV-C1C2 was confirmed by DNA sequencing. The pShuttle-CMV-her3-FL (full-length), pShuttle-Her3ECD, pShuttle-Her3ECDTM and pShuttle-Her3ECDC1C2 were then linearized using digestion with Pme I, recombined into linearized (E1-, E2b-, E3-) serotype 5 pAd construct in BJ 5183 bacterial recombination-based system (Stratagene), and propagated in XL10-Gold Ultracompetent cells (Stratagene). Complementing C7 cell (which express E1 and E2b) were used to produce high titers of these replication-deficient Ad5 vectors, and cesium chloride density gradient was done to purify the Ad5-vectors. All Ad vectors stocks were evaluated for replication-competent adenovirus via PCR-based replication-competent adenovirus assay.

The next generation human HER3 (E1-,E2b- , E3-) Adenovirus vectors are as follows:
1. Ad5 (E2b-)HER3 FL; express human HER3 full length.
2. Ad5 (E2b-)HER3ECDTM; express human HER3 ECD and trans-membrane domain
3. Ad5 (E2b-)HER3ECD; express human HER3 ECD
4. Ad5 (E2b-)HER3ECDC1C2; express human HER3 ECD and C1C2 domain

The ability of each vector to induce a HER3 specific immune responses will be tested, but was expected based on the earlier results and epitopes identified above. Human HER3 specific immune responses to the vectors will be measured in Balb/c mice and in human HER3 transgenic mice.

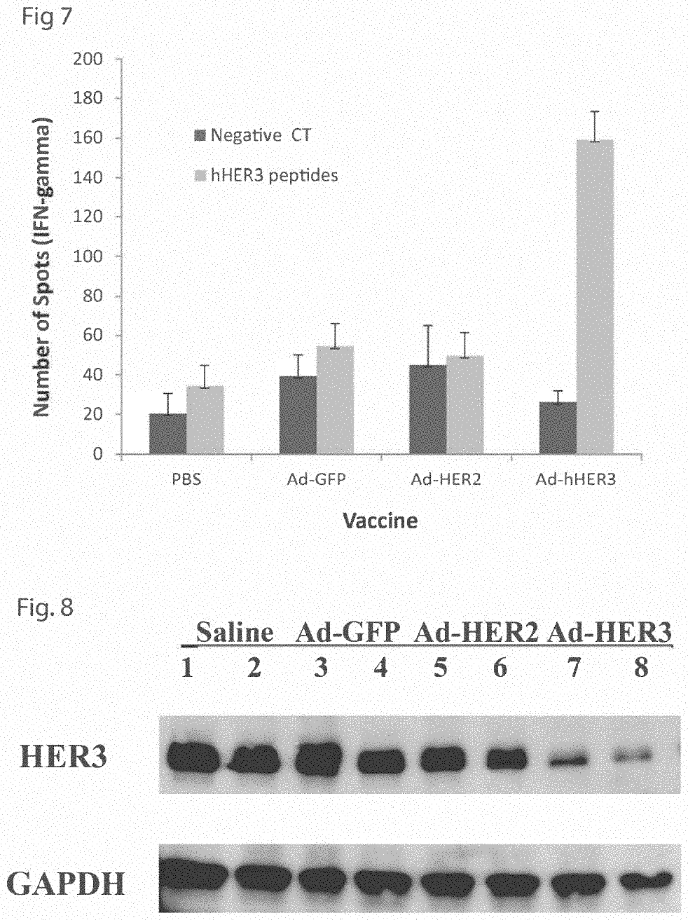

To determine the preventive effect of HER3 vaccination, we have established a HER3 prevention model using JC-HER3 mouse mammary tumor cells in Balb/c mice. As shown in FIG. 6, only vaccination with the HER3 encoding vector prevented growth of the hHER3 expressing tumors in vivo. We next sought to demonstrate development of HER3 specific immune response by ELISPOT. Results are shown in FIG. 7.

Due to the induction of HER3 specific immune responses, we sought evidence whether those tumors that did grow in the HER3 vaccinated mice expressed HER3. In other words, we sought evidence of loss of HER3 in those tumors capable of growth in the vaccinated mice. As shown in FIG. 8, immunization with Ad-hHER3 led to a reduction of HER3 expression in the tumors that did develop. Of interest, immunization with Ad-GFP or Ad-hHER2 did not change HER3 expression.

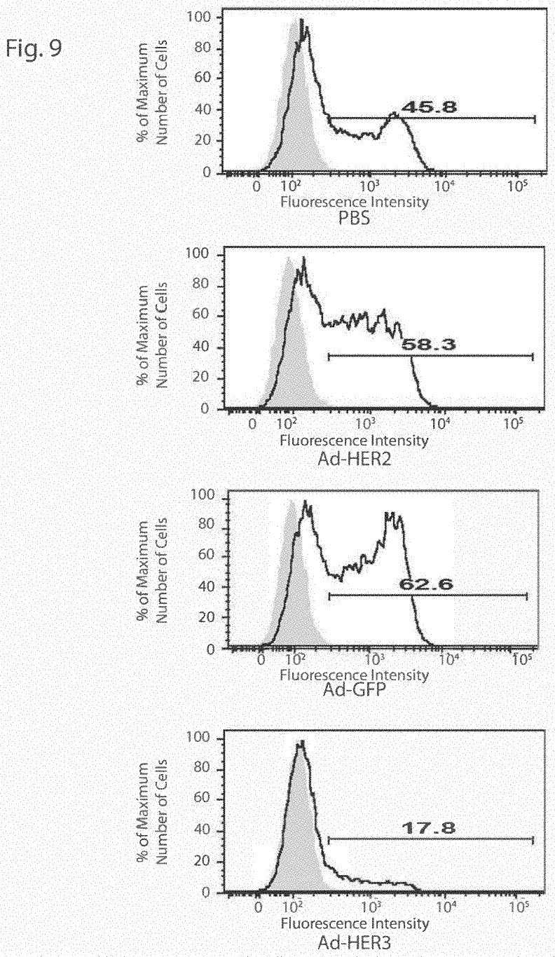

We then tested for surface HER3 expression in the tumors that grew in the HER3 vaccinated mice. As demonstrated in FIG. 9, the surface expression of HER3 was dramatically reduced in the tumors that did grow in the HER3 vaccinated mice.

In summary, we created a HER3 vaccine by generating a recombinant adenovirus encoding human HER3 (Ad-HER3). The Ad-HER3 was highly effective in eliciting significant HER3 specific T-cell and polyclonal antibody responses in mouse models, with the vaccine induced antibodies (VIA) binding multiple HER3 epitopes as well as tumor-expressed HER3 and mediating complement dependent lysis. In addition, the HER3-VIA caused HER3 internalization and degradation, significantly inhibited signaling mediated by receptor heterdimers involving HER3, and retarded tumor growth in vitro and in vivo. Critically, we also showed that the HER3-VIA retarded the growth of human breast cancer refractory to HER2 small molecule inhibitors (lapatinib) in SCID xenografts, providing a compelling argument for the Ad-HER3 vaccine to be tested in patients whose cancer has progressed on HER2 targeted therapy, and in combination with HER2 targeted therapy.

It is interesting to note that the lapatinib-resistant rBT474 clone is much more sensitive to HER3-VIA in vivo than the lapatinib-sensitive BT474M1 clone yet they express equivalent levels of HER3 on the cell surface, which may be a result of increased reliance on HER3 as a driver of tumor growth in the lapatinib resistant BT474 cells. In fact, treatment of the lapatinib resistant BT474 cells leads to decreased HER3, pHER3 and pERK1/2 as expected, but also decreased HER2, pAkt(S473), pS6, p4EPB1, and survivin expression. In contrast, treatment of the lapatinib sensitive BT474 cells with HER3 VIA decreases only HER3, pHER3 and pErk1/2, suggesting that HER3 VIA will have more profound biologic and clinical effects in lapatinib refractory tumors. The lapatinib-resistant BT474 cells also continue to express HER3 protein after treatment with HER3-VIA in vivo, suggesting that antigen loss is not an escape mechanism for lapatinib resistant tumors because HER3 is critical to the tumor survival. Thus, persistent expression of HER3 because of it' role in lapatinib resistance, ensures that tumors will remain targets for vaccine induced T cell and antibody response.

The decrease in the inhibitor of apoptosis protein survivin suggests that a mechanism of resistance to tumor cell killing is also being diminished. We observed similar effects on the expression of survivin in the mouse 4T1-HER2 tumor model which is relatively resistant to trastuzumab, but relatively sensitive to lapatinib. When the 4T1-HER2 expressing tumors were treated with lapatinib or HER2 VIA alone, we observed no change in survivin expression, but when these tumors were treated with a combination of lapatinib and HER2-VIA we observed a decrease in survivin expression, implying that complete HER2 signaling blockade decreased survivin expression. In an analogous fashion, it suggested that complete blockade of HER2:HER3 signaling in lapatinib refractory tumors is accomplished by treatment with HER3-VIA, resulting in the decreased expression of survivin in these studies.

We believe our findings have relevance for counteracting the development of resistance to HER2 targeted therapies. Although HER3 is non-transforming alone, recent data suggests that HER3 expression or signaling is associated with drug resistance to targeted therapies directed against other HER family members. In particular, the acquired resistance to HER2 inhibitors in HER2-amplified breast cancers, trastuzumab resistance in breast cancer, with EGFR inhibitors in lung cancers, with pertuzumab resistance in ovarian cancers, and with EGFR inhibitors in head and neck cancers. The overexpression of HER2:HER3 heterodimers is also negatively correlated with survival in breast cancer. Our approach of targeting HER3 may also have advantages over other HER family targeting strategies. For example, data suggest that trastuzumab is effective against HER1:HER2 heterodimers but not HER2:HER3 heterodimers. HER3 may play a role in therapeutic resistance to other therapies including anti-estrogen therapies in ER positive breast cancers, with hormone resistance in prostate cancers, and with IGF1R inhibitors in hepatomas. Therefore, targeting HER3 may have relevance for counteracting resistance to other pathway inhibitors.

These data suggest that it may be possible to begin a “resistance prophylaxis” vaccination against overexpressed or mutated proteins that will predictably arise to mediate therapeutic resistance, such as HER3. Immunization against these proteins prior to their overexpression as a mediator of therapeutic resistance may avoid immune tolerance induced by their prolonged expression in an immunosuppressive microenvironment. The resulting pre-existing immune response would be much more effective in mediating anti-tumor responses to tumors overexpressing antigen, and/or prevent these mediators from being expressed.

### Example 2

### Vaccination Targeting Human HER3 Alters the Phenotype of Infiltrating T Cells and Responses to Immune Checkpoint Inhibition

### Results

### Adenoviral Vectors Encoding HER3 Elicit Anti-HER3 T Cell and Antibody Responses in HER3-Transgenic Mice

In order to develop a potent, clinically relevant vaccine to induce HER3 specific T and B cell responses, we modified the well-characterized first generation adenovirus serotype 5 vector Ad5[E1-E3-] by inserting the gene for full length human HER3 to generate a viral vector construct referred to subsequently as Ad[E1-]HER3. A recognized challenge with first generation adenoviral vectors is that pre-existing or induced neutralizing antibodies reduce their immunogenicity. Because we have previously demonstrated potent immunogenicity despite anti-vector neutralizing antibodies by using recombinant adenovirus serotype 5 vectors deleted of the early gene E2b in addition to the deletion of E1 and E3 genes (Ad5[E1-E2b-])(29), we generated an Ad5[E1-E2b-] vector expressing full length human HER3 (Ad-HER3-FL). To test the immunogenicity of this HER3 vaccine in the stringent setting where human HER3 is a self-antigen, we first developed a human HER3-transgenic mouse. Further, we crossed the human HER3-transgenic mice to a BALB/c background (F1 Hybrid mice; BALB/c x MMTV-neu/MMTV-hHER3) and created a new human HER3 expressing tumor model based on the BALB/c-derived JC murine breast cancer cell line (JC-HER3).

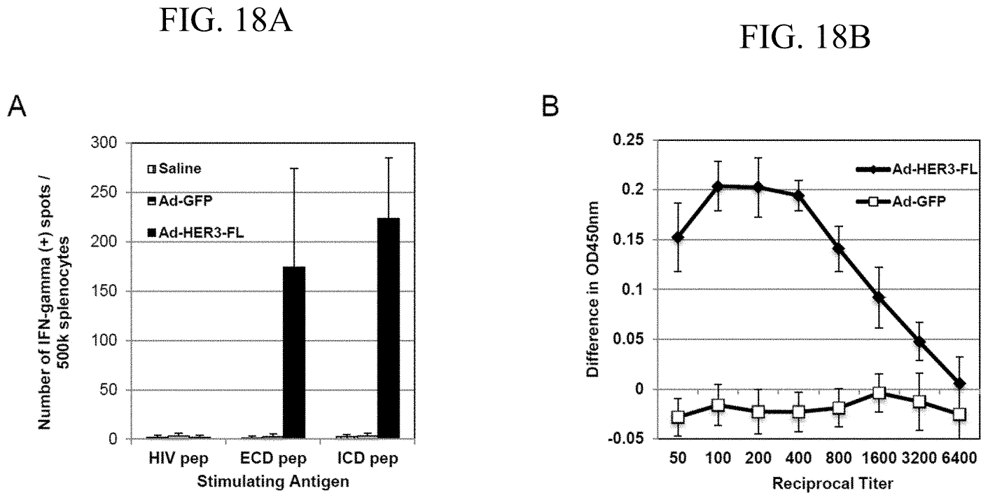

These human HER3-transgenic mice were immunized with the Ad-HER3-FL vector following which their splenocytes were analyzed for HER3-specific cellular immune responses by the IFN-gamma ELISPOT assay. FIG. 18A demonstrates an equally strong cellular response against epitopes from the HER3 extracellular domain (ECD) and intracellular domain (ICD) following Ad-HER3-FL vaccination. The vaccine also induced an anti-HER3 antibody response as measured by the binding of serum polyclonal antibodies to human HER3-transfected 4T1 murine breast cancer cells (4T1-HER3) compared with antibody binding after control Ad-GFP vaccination, FIG. 18B.

### Ad-HER3 Immunization Reduces Growth of Established HER3+ Breast Cancer

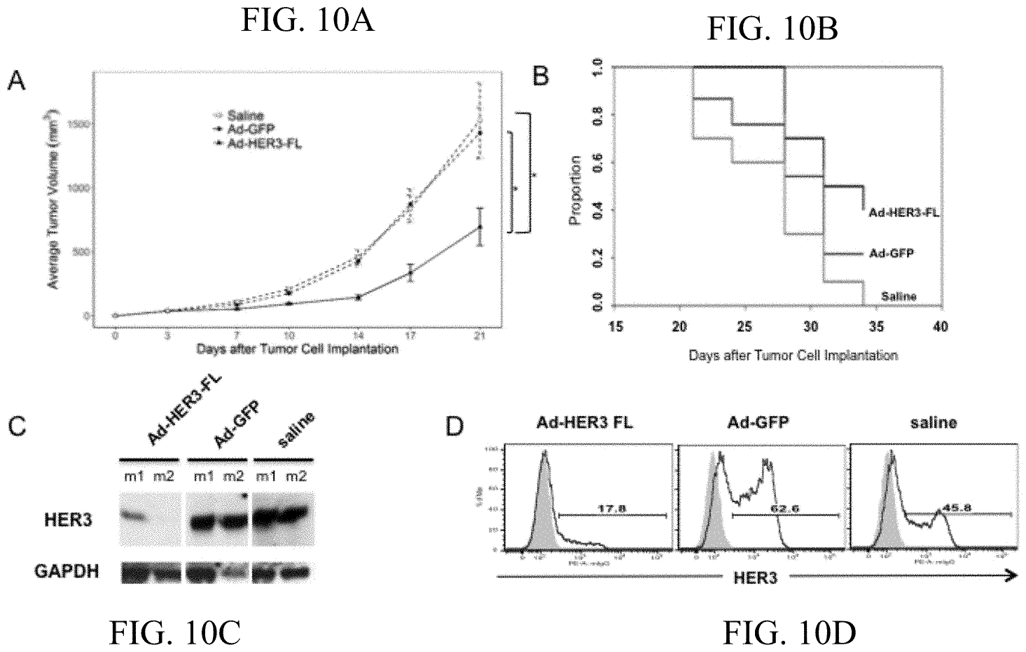

We tested the anti-tumor effects of vaccination with the Ad-HER3-FL construct in therapeutic models following JC-HER3 tumor cell implantation. We found that the Ad-HER3-FL vaccine effectively suppressed JC-HER3 tumor growth compared to the controls, specifically saline (p<0.001), and an irrelevant vaccine, Ad-GFP (p<0.001) (FIG. 10A), and this was associated with improved survival compared to saline treatment (p=0.005) (FIG. 10B) and demonstrated a trend toward improved survival when compared to the Ad-GFP vector, though we did not observe any tumor regression with Ad-HER3-FL vaccination.

In order to investigate potential sources for tumor escape from the HER3-specific immune response, we first analyzed tumor expression of HER3. In this model of HER3 immunotherapy, tumor expression of HER3 is not critical to maintaining the malignant phenotype. Therefore, one mechanism of immune escape in the presence of HER3 specific T cells and anti-HER3 antibodies would be HER3 antigen loss. We performed western blot on tumor lysates and flow cytometry on tumor cells remaining 21 days after the first vaccination. As shown in FIG. 10C, tumors from mice immunized with the Ad-HER3-FL vaccine, have down-regulation of HER3 expression, but it is not completely lost in all Ad-HER3-FL vaccinated mice. Similarly, on flow cytometric analysis, HER3 decreased but some HER3 expression persisted after Ad-HER3-FL vaccination (FIG. 10D). These data demonstrate that one mechanism of escape is antigen down regulation but it is not the only explanation.

Ad-HER3-FL Vaccination Increases T Cell Infiltration into Tumors

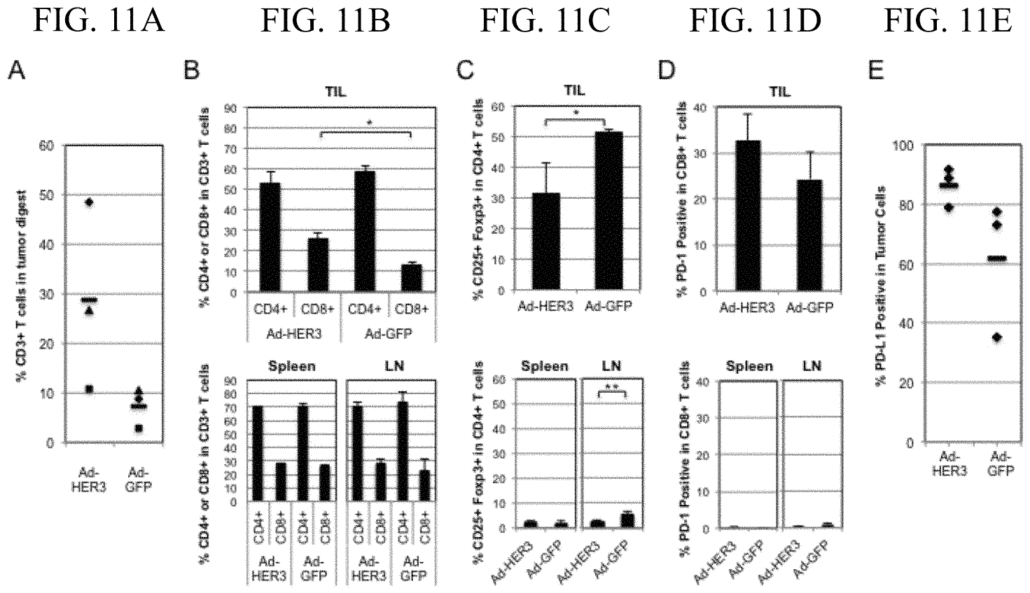

We sought to evaluate other potential explanations of tumor progression despite robust T cell responses against HER3. First we wished to determine if there was T cell infiltration of tumor by analyzing tumor infiltrating lymphocytes (TIL) in all vaccinated mice and found a greater number of CD3+ TILs in Ad-HER3-FL immunized mice compared to the Ad-GFP immunized mice (FIG. 11A). Among these TILs, there was a greater percentage of CD8+ (p<0.05) but not CD4+ TILs in the Ad-HER3-FL immunized mice. In contrast, there was no difference in the CD4+ and CD8+ T cell content within splenocytes or distant (non-tumor draining) lymph nodes in these Ad-HER3-FL vaccinated mice (FIG. 11B).

Other proposed mechanisms for immunosuppression involve the presence of regulatory T cells (Treg). We noted fewer intratumoral Tregs in the Ad-HER3-FL vaccinated mice compared to the Ad-GFP treated mice, p=0.026 (FIG. 11C), resulting in a greater intratumoral CD8+ to Treg ratio (data not shown). These data suggest that the immunosuppression did not involve activation of Tregs by the vaccine. Another well-established immunosuppressive mechanism is the presence of PD-1 on activated T cells.

Analysis of PD-1 expression on TILs, splenocytes and distant (non-tumor draining) lymph nodes after Ad-HER3-FL or Ad-GFP vaccination confirmed that PD-1 tended to be overexpressed by CD8+ TILs after Ad-HER3-FL vaccination compared to PD-1 expression by CD8+ T cells isolated from splenocytes and non-tumor draining lymph nodes in these same mice (FIG. 11D). Similarly, we noted a trend for higher tumor cell PD-L1 expression after Ad-HER3-FL vaccination compared to control, FIG. 11E. These data suggest that activated TILs induced by Ad-HER3-FL vaccination are at risk of being suppressed through the PD-1/PD-L1 signaling axis due to both tumor PD-L1 expression and their own high PD-1 expression.

Enhanced Antitumor Activity with Checkpoint Blockade Plus Ad-HER3 Vaccine

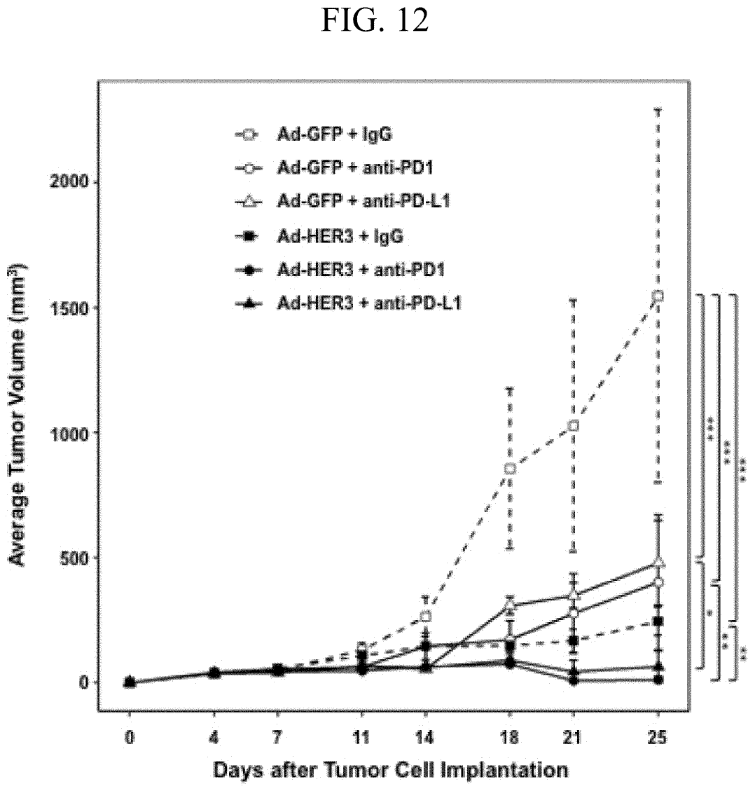

In order to study the functional consequences of PD-1 expression by intratumoral T cells, we tested whether blockade of the PD-1/PD-L1 interaction in combination with Ad-HER3-FL immunizations would have greater anti-tumor efficacy than either alone. We first evaluated this effect in a tumor prevention model. In this model, mice were first immunized with the Ad-HER3-FL vaccine, tumor was then implanted, and tumor implantation was followed by anti-PD-1 or anti-PD-L1 antibody administration. While Ad-HER3-FL alone or anti-PD-1 or anti-PD-L1 with control vector resulted in some delayed tumor growth, there was no tumor regression (FIG. 12). In contrast, vaccination with Ad-HER3-FL prior to tumor implantation followed by either anti-PD-L1 or anti-PD-1 antibodies after tumor implantation induced tumor regression (p<0.01 for the comparison of Ad-HER3-FL+IgG versus Ad-HER-FL+anti-PD1; p<0.01 for the comparison of Ad-HER3-FL+IgG versus Ad-HER3-FL+anti-PD-L1).

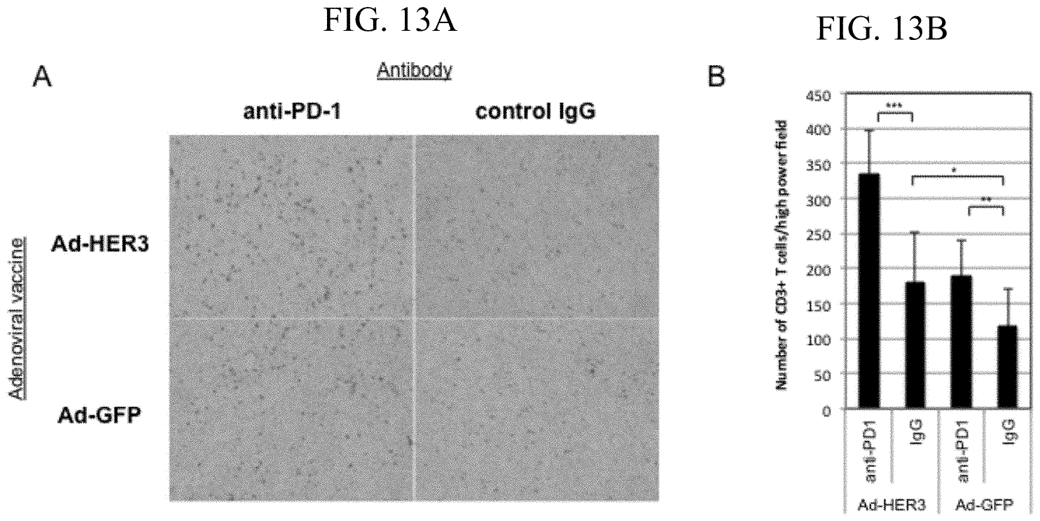

We next wanted to determine whether tumor regression was due to the modulation of the intratumoral T cell infiltrate by checkpoint blockade after vaccination in the prevention model. The addition of anti-PD-1 antibodies to Ad-HER3-FL vaccination significantly increased the number of CD3+ T cells/hpf within the tumor compared to Ad-HER3-FL vaccination alone (p<0.0001) (FIGS. 13A, 13B). Interestingly, there was an increase in the T cell infiltrate caused by anti-PD-1 antibody treatment regardless of whether the anti-PD-1 antibody was administered with either Ad-HER3-FL or Ad-GFP. However, the combination of Ad-HER3-FL plus anti-PD-1 antibody induced the greatest T cell infiltrate/hpf.

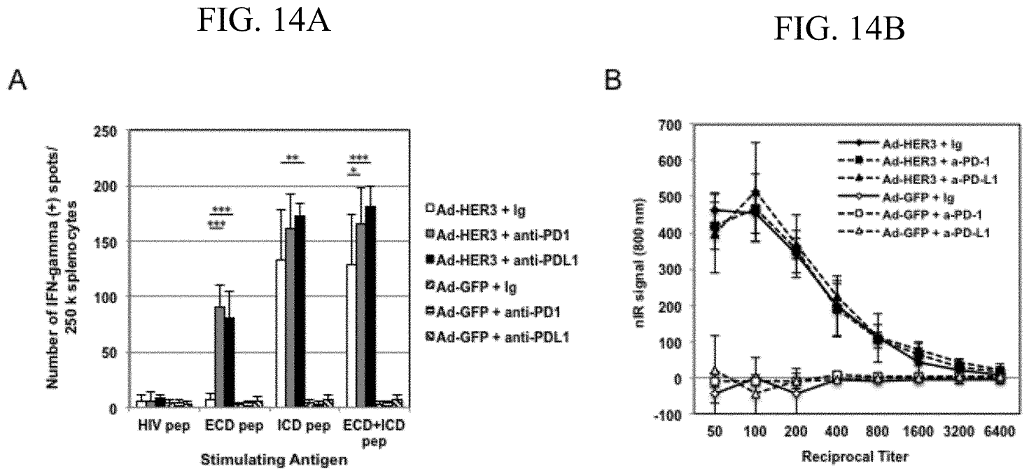

We next interrogated if anti-PD-1 treatment could augment the magnitude of both the HER3-specific T cell and anti-HER3 antibody response induced by Ad-HER3-FL alone. In the prevention model, splenocytes from mice treated with either the Ad-HER3-FL vaccine, Ad-HER3-FL vaccine+anti-PD-1 antibody, or Ad-HER3-FL vaccine+anti-PD-L1 antibody demonstrated an increased frequency of T cells specific for HER3 ECD and ICD peptides (FIG. 14A). However, neither anti-PD-1 nor anti-PD-L1 antibodies given with control vaccine affected the serum titer of anti-HER3 antibodies induced by Ad-HER3-FL-immunization (FIG. 14B). These data support a role for PD-1/PD-L1 blockade as an additional strategy to further increase antigen-specific T cell activation induced by Ad-HER3-FL vaccination.

### Combination of Checkpoint Blockade and Ad-HER3-FL has Enhanced Anti-Tumor Activity in Tumor Bearing Mice

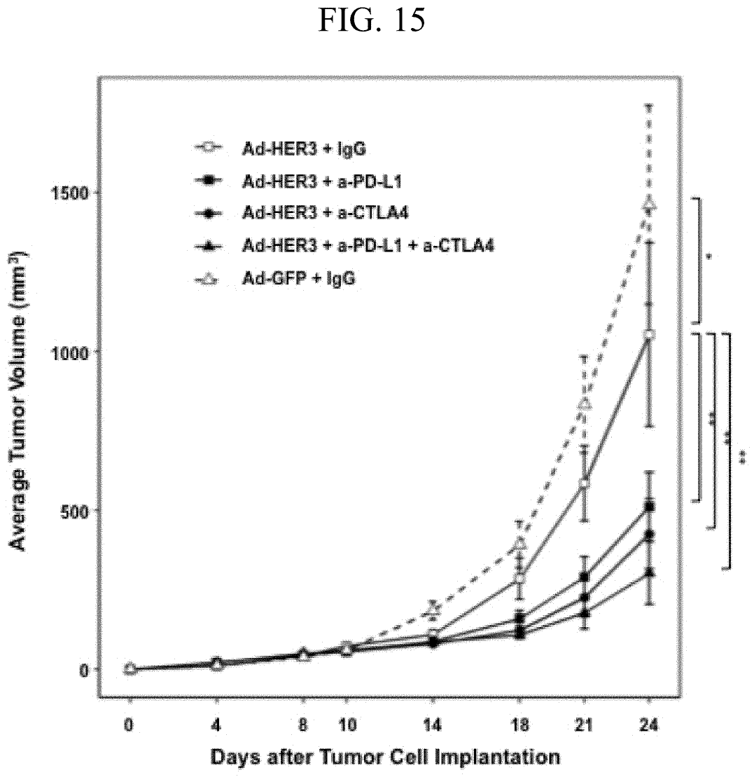

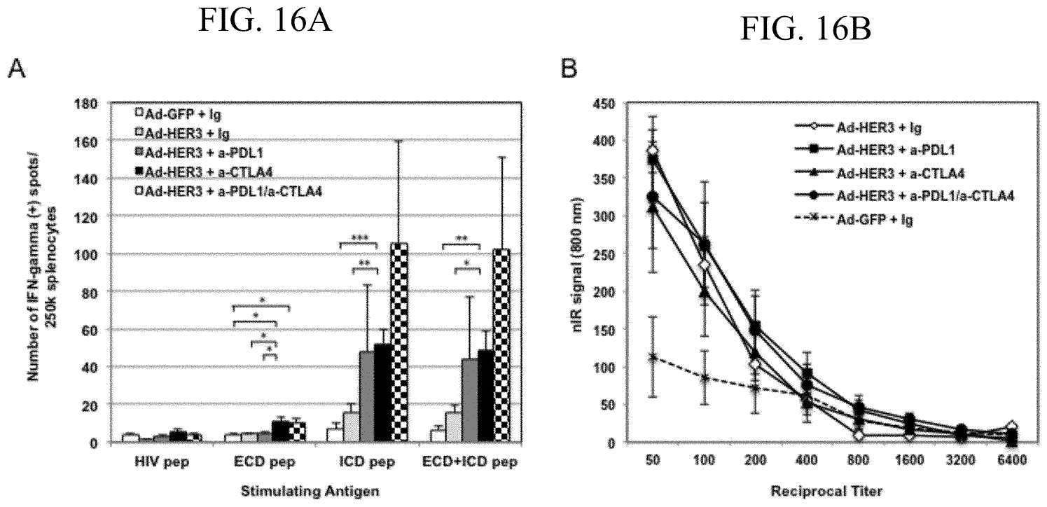

Having demonstrated that checkpoint blockade enhanced the anti-tumor activity of the Ad-HER3-FL in the less stringent prevention model, we wished to evaluate the efficacy of these antibodies in enhancing the anti-tumor activity of Ad-HER3-FL immunization in tumor-bearing mice (treatment model). We focused on anti-PD-L1 and anti-CTLA4 in these experiments. HER3 transgenic mice implanted with JC-HER3 cells were vaccinated with Ad-HER3-FL or control Ad-GFP simultaneously with anti-PD-L1, anti-CTLA4 or both. There was slowing of tumor growth by Ad-HER3-FL plus either antibody alone (p<0.001, for both comparisons) or with the combination of both antibodies (p<0.001) compared with Ad-HER3-FL alone (FIG. 15). Analysis of splenocytes from this experiment suggested that anti-PD-L1 or anti-CTLA4 or their combination plus the HER3 vaccine increased the magnitude of HER3-specific T cell response compared with vaccine alone (FIG. 16A). Furthermore, there was no apparent difference in the titer of antibodies induced with Ad-HER3 vaccine with or without the addition of the checkpoint antibodies (FIG. 16B).

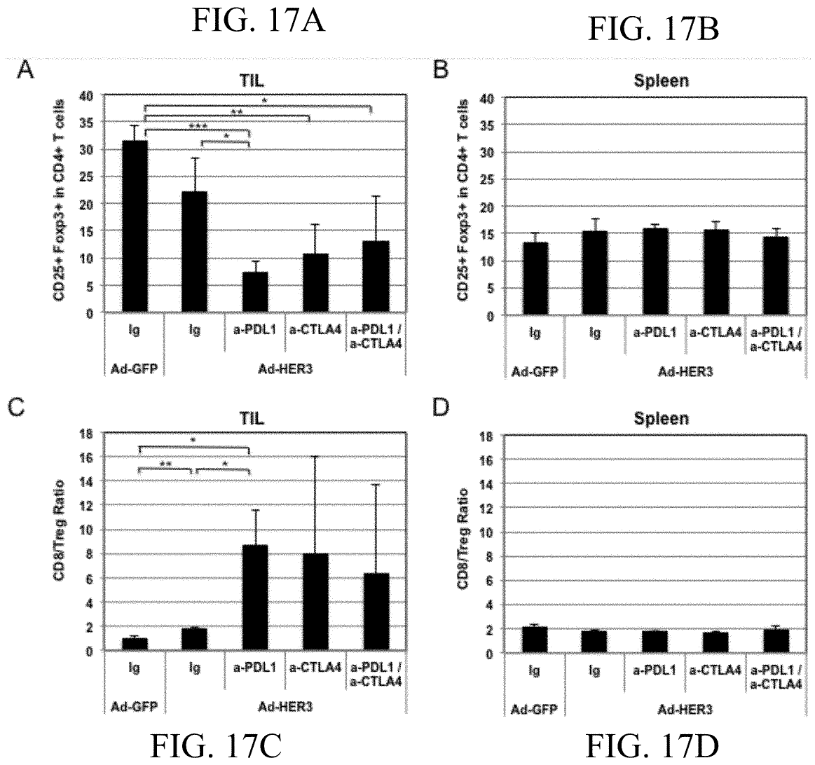

Further, each antibody and their combination when administered with the Ad-HER3-FL vaccine, decreased intratumoral Treg content (FIG. 17A) and increased CD8 to Treg ratio (FIG. 17C) in established tumors compared with Ad-HER3-FL alone. In contrast, there was no significant difference in the splenic Treg content (FIG. 17B) or CD8 to Treg ratio (FIG. 17D) when comparing the different treatment conditions, suggesting that the effect of the checkpoint antibodies occurs at the site of the tumor.

### Discussion

HER3 mediates resistance to EGFR-, HER2- and endocrine-directed therapies in breast cancer and other epithelial malignancies, but has been challenging to target. Our initial objective was to develop a vaccine capable of inducing HER3-specific immune effectors, which would have anti-tumor efficacy against resistant tumors. We chose an adenoviral backbone deleted of the E1 and E2b genes that we previously demonstrated in clinical studies to activate immune responses against the encoded transgene despite the development of anti-Ad neutralizing antibody (30). We developed a model of human HER3 expressing murine breast cancer (JC-HER3) implantable into immune competent human HER3 transgenic mice to test the adenoviral vaccines. The E1, E2b-deleted vector induced T cells with specificities against both intracellular and extracellular domains of HER3 in HER3-transgenic mice. The Ad-HER3 vaccine also demonstrated the ability to modulate the immune cell content of tumors. Specifically, Ad-HER3 vaccination resulted in an increased percentage of intratumoral CD8 T cells and a decreased percentage of intratumoral Tregs, yielding an increased CD8 to Treg ratio, a trend favorable for inducing immune mediated anti-tumor activity. This resulted in a delay in tumor growth; however, we wished to develop a strategy that led to greater tumor regression.

One strategy to enhance the antitumor activity of the vaccine was suggested by the observation that although the Ad-HER3-FL immunization caused an increase in TILs compared to control immunizations, these TILs demonstrated high expression of PD-1 compared with splenocytes or T cells from non-tumor draining lymph nodes. It has been previously suggested that T cells specific for a vaccinating antigen upregulate PD-1.(30, 31) As the PD-1/PD-L1 interaction is well established to impair T cell-mediated anti-tumor activity, we sought to enhance the anti-tumor activity of the Ad-HER3-FL vaccine by blocking the PD-1/PD-L1 interaction. Indeed, there was elimination of tumor when we immunized mice with the Ad-HER3-FL prior to tumor implantation and then delivered the anti-PD-1 or anti-PD-L1 antibody after tumor implantation. In this setting, there was sufficient time to generate a robust intratumoral antigen specific immune response which could be further enhanced by checkpoint blockade. The robust immune response generated by vaccination before tumor cell implantation may model the clinical scenario of vaccination of patients with resected tumors at high risk of recurrence. In this setting if tumor were to recur, anti-PD-1/PD-L1 blockade may lead to tumor regression because of the presence of intratumoral T cells activated by previous vaccination. This may also model the clinical scenario of tumors controlled by standard therapy, which then grow upon development of resistance due to upregulation of molecules such as HER3. In this setting, tumors that upregulate HER3 and contain infiltrates with HER3 specific T cells would be rapidly eliminated upon application of PD-1/PD-L1 blockade.

In contrast to the prevention model, vaccination therapies of established malignancies have had modest success in pre-clinical and clinical testing; as other groups have reported greater anti-tumor activity for vaccines combined with PD-1/PD-L1 blockade in murine treatment models (32-34), we wished to test the administration of PD-1/PD-L1 blockade with Ad-HER3-FL in established tumors. In the stringent treatment models, there was slowing of tumor growth with either PD-1/PD-L1 blockade. We reasoned that in treatment models, there would be little time for a T cell response following vaccination alone to achieve a frequency necessary to eradicate tumor. Therefore, we also tested the addition of anti-CTLA4 to determine if this alone or in conjunction with PD-1/PD-L1 blockade could cause rapid T cell expansion after vaccination.

In poorly immunogenic tumor models, it has been demonstrated that anti-CTLA4 therapy strongly enhances the amplitude of vaccine induced anti-tumor activity (35, 36). We observed in the treatment model that anti-CTLA4 or blockade of the PD-1/PD-L1 interaction (anti-PD-L1) and their combination plus the Ad-HEr3 vaccine similarly enhanced immune-mediated tumor control.

Our data suggest that current cancer vaccine strategies would be enhanced by checkpoint blockade. Single and dual checkpoint blockade appear to enhance anti-tumor response to the Ad-HER3 vaccine similarly. Therefore, the choice of checkpoint antibody may depend more on their indication. For example, if single agent checkpoint blockade is the standard therapy for a malignancy where HER3 would also be relevant (e.g. triple negative breast cancer), then combining the HER3 vaccine with the standard single agent checkpoint blockade antibody would be appropriate. However, where dual checkpoint is the standard, our data suggest that this leads to similar enhancement in anti-tumor activity to the HER3-FL vaccine.

Our data now warrant clinical testing of the Ad-HER3-FL vaccine with anti-PD-1/PD-L1, anti-CTLA4 therapy, or both in the setting of established malignancy and with anti-PD-1 or anti-PD-L1 antibodies in the adjuvant setting. As our pre-clinical testing has demonstrated minimal side effects from this vaccine, we anticipate that our planned first-in-human clinical trial of this vaccine will be well tolerated. A phase I study of the Ad-HER3 full-length vaccine will open shortly in order to evaluate the safety and immunogenicity of this vaccine in metastatic cancer patients with a planned expansion cohort for hormone receptor positive breast cancer. As HER3 is recognized to mediate anti-HER2 therapy resistance, we plan to open a clinical trial of the Ad-HER3 vaccine given in combination with anti-HER2 therapy in metastatic HER2+ breast cancer. Our prior studies have also revealed that in HER2+ breast cancer, activation of the HER3 signaling axis is associated with a poor outcome (37). Lastly, there is increasing evidence that single agent check point blockade is clinically active in a portion of TNBC patients (38, 39). In addition, there is evidence that HER3 expression is associated with worse DFS and OS in TNBC (40). Based on these observations, we will open a trial of concurrent Ad-HER3 vaccination and check point blockade in TNBC to assess the safety and immunogenicity of this combination therapy.

### Materials and Methods

### Adenoviral Vector Preparation

The human HER3 cDNA was excised from a pCMVSport6-HER3-HsIMAGE6147464 plasmid (cDNA clone MGC:88033/IMAGE:6147464) from the ATCC (Manassas, Va.). Construction of a first-generation [E1-, E3-] Ad vector containing human full length HER3 under control of human CMV promoter/enhancer elements was performed using the pAdEasy system (Agilent technologies, Santa Clara, Calif.) as previously described (41). The modified adenoviral vector, [E1-,E2b-] Ad, was constructed as previously described (42). This vector has multiple deletions of the early region 1 (E1) and E2b regions (DNA polymerase and pTP genes), and was engineered to express the identical human CMV promoter/enhancer-transgene cassette as utilized for the [E1⁻E3⁻] Ad-HER3 vector. Ad[E1-E2b-]-HER3 FL vector was constructed with full length of HER3 cDNA. Complementing C-7 cell lines were used to support the growth and production of high titers of these vectors, and cesium chloride double banding was performed to purify the vectors, as previously reported (43).

### Reagents and Peptides

Mixtures of HER3 peptides containing 15mer peptides, each overlapping the next by 11 amino acids, spanning extracellular domain plus transmembrane segment (ECD-TM) of HER3 protein and intracellular domain (ICD) of HER3 protein, were purchased from JPT Peptide Technologies (Berlin, Germany), and were used for the IFN-γ ELISPOT assay. An HIV peptide mix representing HIV gag protein was purchased from JPT Peptide Technologies (Berlin, Germany) and was used as a negative control. Anti-murine PD-1 (BE0146, clone J43) and anti-murine PD-L1 (BE0101, clone 10F.9G2) and anti-murine CTLA4 (BE0164, clone 9D9) monoclonal antibodies were purchased from Bio X Cell (West Lebanon, N.H.) for animal experiments. Collagenase III (cat# 4183) was purchased from Worthington Biochemical (Lakewood, N.J.), and hyaluronidase (H3884) and DNase (D5025) from Sigma-Aldrich (St. Louis, Mo.).

### Mice

Female wild-type BALB/c mice (Jackson Laboratory, Bar Harbor, Me., USA) were bred and maintained in the Duke University Medical Center pathogen-free Animal Research Facility, and used at 6 to 8 weeks of age. Human HER3-transgenic mice (MMTV-neu/MMTV-hHER3) with FVB background were a kind gift from Dr. Stan Gerson at Case Western Reserve University. FVB mice homozygous for the HER3 gene were established at Duke University and crossed with BALB/c mice to generate F 1 hybrid HER3 transgenic mice (FVB×BALB/c) for use in tumor implantation experiments. All animal studies described were approved by the Duke University Medical Center Institutional Animal Care & Use Committee and the US Army Medical Research and Materiel Command (USAMRMC) Animal Care and Use Review Office (ACURO) and performed in accordance with guidelines published by the Commission on Life Sciences of the National Research Council.

### Detecting HER3 Expression by Western Blotting

Tumor tissues were collected at the termination of animal experiments and minced and homogenized in RIPA buffer in the presence of proteinase inhibitors. After centrifugation at 13,000 rpm for 10 min at 4° C., the supernatant was pooled, filtered through a 0.22 μm filter, aliquoted and stored at −80° C. until needed. Protein concentration was determined by a BCA assay. Thirty μg of protein was applied for each lane, run on 12% Tris-HCl acrylamide gel, and transferred to polyvinylidene fluoride (PVDF) membranes. Membranes were incubated with anti-HER3 antibody (1:1000 dilution, Santa Cruz Biotechnology, Inc., Santa Cruz, Calif.) or anti-GAPDH antibody (1:1000 dilution, Santa Cruz) for 1 h, followed by incubation with horseradish peroxidase-conjugated goat anti-mouse IgG antibody (1:2000 dilution, Bio-Rad, Hercules, Calif.). The chemiluminescent substrate kit (Thermo Scientific, Rockford, Ill.) was used for the development.

### Flow Cytometry of Tumor Infiltrating Lymphocytes and Tumor HER3 Expression

Tumors were excised from mice at the termination of tumor implantation experiments, minced with surgical blades and digested with triple enzyme buffer (collagenase III, hyaluronidase, DNase) for 1.5 hours at 37° C. The cell suspension was washed 3 times with PBS and resuspended in PBS. Cells were first labeled with viability dye (Fixable Aqua Dead Cell Stain Kit, Invitrogen, Eugene, Oreg.) for 5 min, and then with PerCP/Cy5.5-anti-CD3, APC/Cy7-anti-CD8, Alexa Fluor 700-anti-CD4, FITC-anti-CD25, APC-anti-PD-1, and PE-anti-PD-L1 or PE-anti-HER3 antibody (BioLegend, San Diego, Calif.) for 30 min at 4° C. Cells were washed twice with PBS and analyzed on a LSRII machine (BD Biosciences) using FlowJo software.

### IFN-γ Enzyme-Linked Immunosorbent Spot (ELISpot) Assay

Mouse IFN-γ ELISPOT assay (Mabtech Inc., Cincinnati, Ohio) was performed according to the manufacturer's instructions. At the end of the mouse experiments, their spleens were collected and lymphocytes were harvested by mincing and passing through a 40 μm Cell Strainer. Red blood cells were lysed with red blood cell lysis buffer (Sigma). Splenocytes (500,000 cells/well) were incubated in RPMI-1640 medium (Invitrogen) supplemented with 10% horse serum, and HER3 ECD-TM peptide mix and/or HER3 ICD peptide mix (1.3 μg/ml) were used as stimulating antigens. HIV peptide mix was used as a negative control, and a mixture of PMA (50 ng/ml) and Ionomycin (1 μg/ml) was used as a positive control for the assay. Membranes were read with a high-resolution automated ELISpot reader system (Carl Zeiss, Inc., Thornwood, N.Y., USA) using the KS ELISpot version 4.2 software.

### Cell-Based ELISA

4T1 cells were transduced with HER3 gene by lentiviral vectors to express human HER3 on the cell surface (4T1-HER3 cell). 4T1 and 4T1-HER3 cells were incubated overnight at 37° C. in 96 well flat bottomed plates (3×10⁴ cells in 100 μL medium/well). Mouse sera were prepared by diluting with DMEM medium (final titrations 1:50˜1:6,400), and 50 μl of mouse sera-containing media were added to the wells and incubated for 1 hour on ice. The plates were gently washed with PBS twice, and then, cells were fixed with diluted formalin (1:10 dilution of formalin in 1% BSA in PBS) for 20 min at room temperature. After washing three times with PBS, 50 μL of 1:2000 diluted HRP-conjugated goat anti-mouse IgG was added to the wells, and incubated for 1 h at room temperature. After washing three times with PBS, TMB substrate was added to the wells (50 μl/well) and incubated for approximately 20 min. The color development was stopped by adding 50 μ1 of 1M H2504 buffer. Absorbance at 450 nm was read using a BioRad Microplate Reader (Model 680). As the alternative method for the detection of HER3-specific antibody, near infrared red (nIR) dye-conjugated anti-mouse IgG (IRDye 800CW, LI-COR Biosciences, Lincoln, Nebr.) was used as a secondary antibody, and the nIR signal was detected by a LI-COR Odyssey Imager (LI-COR) using the 800 nm channel.

### Prophylactic Anti-Tumor Model in HER3-Transgenic Mice

HER3-transgenic F1 hybrid mice were immunized by footpad injection on days -11, -4 and 14 with 2.6×10¹⁰ particles of the Ad[E1-,E2b-]-HER3-FL or Ad-GFP control in 40 μμL of saline. On day 0, mice were inoculated with 5×10⁵ JC-HER3 cells in 100 μl saline subcutaneously into the flank. Tumor dimensions were measured serially, and tumor volumes calculated using the following formula: long axis×(short axis)×0.5. For the combination treatment with immune checkpoint inhibitors, mice were vaccinated with Ad-HER3-FL or Ad-GFP on days -11, -4 and 14, and received peritoneal injection of anti-PD-1 antibody, anti-PD-L1 antibody or control IgG (200 μg/injection) twice a week (on days 3, 6, 10, 13, 17 and 20) after tumor implantation.

### Therapeutic Anti-Tumor Model in F1 Hybrid HER3 Transgenic Mice

HER3 transgenic F1 hybrid mice were inoculated with 5×10⁵ JC-HER3 cells in 100 μL saline subcutaneously into the flank on day 0. On days 3 and 10, mice were immunized via footpad injection with Ad-HER3-FL or Ad-GFP control vector (2.6×10¹⁰ particles/mouse for each injection). Tumor dimensions were measured serially and tumor volumes were calculated as described above. Mice were euthanized when the tumor size reached the humane endpoint, or by day 34. For the combined treatment with immune checkpoint inhibitors (anti-PD-1, anti-PD-L1, or anti-CTLA4 antibody), mice received peritoneal injection of the checkpoint inhibitor (200 μg/injection) twice a week after tumor implantation.

### Tissue Analysis of Tumor-Infiltrating T Cells

Tumor tissue collected at the time mice were euthanized was fixed in 10% neutral buffered formalin for a minimum of 24 hours. The tissue was then processed and embedded in paraffin. Sections with 5 μm thickness were made for hemotoxylin and eosin staining and CD3+ T cell staining. For immunohistochemistry using anti-CD3 antibody (Thermo Fisher Scientific, Waltham, Mass.), heat-induced antigen retrieval was performed using sodium citrate buffer for 20 min after deparaffinization of tissue sections. Following quenching of endogenous peroxidase activity with 3% H₂O₂, 10% normal horse serum was used to block nonspecific binding sites. Anti-CD3 antibody (1:150 dilution) was applied to the sections, which were incubated overnight at 4° C. After three washes with PBS, anti-rabbit IgG secondary antibody (ImmPRESS anti-Rabbit IgG Polymer, Vector Lab, Burlingame, Calif.) was applied for 30 min, and then color was developed using the DAB Peroxidase substrate kit (Vector Lab).

Counterstaining was performed with hematoxylin. After assessment of adequate staining by two independent observers, ten high power fields (magnification ×200; objective lens ×20, ocular ×10) of tumor tissue for each group, avoiding necrotic area, were randomly selected and photographed using an IX73 Inverted Microscope with Dual CCD Chip Monochrome/Color Camera (Olympus). CD3-positive spots were counted for each field by two observers who had no previous knowledge of treatments performed for individual groups.

### Statistical Analysis

For the ELISpot and ELISA assays, differences in IFN-γ production and antibody binding, respectively, were analyzed using the Student's t test. Tumor volume measurements for in vivo models were analyzed under a cubic root transformation to stabilize the variance. Welch t-tests were used to assess differences between mice injected with HER3-VIA or control GFP-VIA.

To compare tumor growth volumes over time, a multivariable Generalized Additive Model for Location, Scale and Shape (GAMLSS) (44) considering Group, Experiment, Time and interaction between Time and Group as covariables for Tumor Volume location and Time for Tumor Volume scale was applied. The Normal distribution was considered for the effectiveness of Ad-HER3 FL vaccine model and the Zero Adjusted Gamma distribution for the effectiveness of antibodies model. Time was modeled using penalized cubic spline (45) and the interaction between Time and Group was modeled using Varying Coefficient (46). Areas under tumor growth curve were calculated under spline interpolation (44) and adaptive quadrature for the tumor prevention model. A simple GAMLSS with Gamma distribution quantifies the relationship between mean of area under tumor growth curve and the covariable Group.

Contrasts were calculated using the Wald statistic and multiples comparisons were corrected as suggested by Holm (47). Model diagnostics was performed based on Worm-plots (48) and fitted values were compared considering 95% Bootstrap Confidence Intervals (49).

The Kaplan-Meier method was used to estimate overall survival and treatments were compared using a two-sided log-rank test. Analyses were performed using R version 2.10.1, SAS v. 9.3 (SAS Institute, Cary N.C.) and R, version 3.2.5 (50), survival plots were created using Spotfire S+ v. 8.1 (TIBCO, Palo Alto, Calif.). All tests of hypotheses will be two-sided considering a significance level of 0.05.

### References:

1. Roskoski R, Jr. The ErbB/HER family of protein-tyrosine kinases and cancer. Pharmacological research : the official journal of the Italian Pharmacological Society. 2014;79:34-74.

2. Cai Z, Zhang H, Liu J, Berezov A, Murali R, Wang Q, et al. Targeting erbB receptors. Seminars in cell & developmental biology. 2010;21(9):961-6.

3. Takikita M, Xie R, Chung J Y, Cho H, Ylaya K, Hong S M, et al. Membranous expression of Her3 is associated with a decreased survival in head and neck squamous cell carcinoma. J Transl Med. 2011;9:126.

4. Chiu C G, Masoudi H, Leung S, Voduc D K, Gilks B, Huntsman D G, et al. HER-3 overexpression is prognostic of reduced breast cancer survival: a study of 4046 patients. Annals of surgery. 2010;251(6):1107-16.

5. Hayashi M, Inokuchi M, Takagi Y, Yamada H, Kojima K, Kumagai J, et al. High expression of HER3 is associated with a decreased survival in gastric cancer. Clin Cancer Res. 2008;14(23):7843-9.

6. Giltnane J M, Moeder C B, Camp R L, Rimm D L. Quantitative multiplexed analysis of ErbB family coexpression for primary breast cancer prognosis in a large retrospective cohort. Cancer. 2009;115(11):2400-9.

7. Begnami M D, Fukuda E, Fregnani J H, Nonogaki S, Montagnini A L, da Costa WL, Jr., et al. Prognostic implications of altered human epidermal growth factor receptors (HERs) in gastric carcinomas: HER2 and HER3 are predictors of poor outcome. J Clin Oncol. 2011;29(22):3030-6.

8. Reschke M, Mihic-Probst D, van der Horst EH, Knyazev P, Wild P J, Hutterer M, et al. HER3 is a determinant for poor prognosis in melanoma. Clin Cancer Res. 2008;14(16):5188-97.

9. Lee C H, Huntsman D G, Cheang M C, Parker R L, Brown L, Hoskins P, et al. Assessment of Her-1, Her-2, And Her-3 expression and Her-2 amplification in advanced stage ovarian carcinoma. Int J Gynecol Pathol. 2005;24(2):147-52.

10. Kol A, Terwisscha van Scheltinga A G, Timmer-Bosscha H, Lamberts L E, Bensch F, de Vries E G, et al. HER3, serious partner in crime: therapeutic approaches and potential biomarkers for effect of HER3-targeting. Pharmacol Ther. 2014;143(1):1-11.

11. Sergina N V, Rausch M, Wang D, Blair J, Hann B, Shokat K M, et al. Escape from HER-family tyrosine kinase inhibitor therapy by the kinase-inactive HER3. Nature. 2007;445(7126):437-41.

12. Frogne T, Benjaminsen R V, Sonne-Hansen K, Sorensen B S, Nexo E, Laenkholm A V, et al. Activation of ErbB3, EGFR and Erk is essential for growth of human breast cancer cell lines with acquired resistance to fulvestrant. Breast Cancer Res Treat. 2009;114(2):263-75.

13. Musgrove E A, Sutherland R L. Biological determinants of endocrine resistance in breast cancer. Nat Rev Cancer. 2009;9(9):631-43.

14. Tovey S, Dunne B, Witton C J, Forsyth A, Cooke T G, Bartlett J M. Can molecular markers predict when to implement treatment with aromatase inhibitors in invasive breast cancer? Clin Cancer Res. 2005;11(13):4835-42.

15. Arpino G, Wiechmann L, Osborne C K, Schiff R. Crosstalk between the estrogen receptor and the HER tyrosine kinase receptor family: molecular mechanism and clinical implications for endocrine therapy resistance. Endocr Rev. 2008;29(2):217-33.

16. Liu B, Ordonez-Ercan D, Fan Z, Edgerton S M, Yang X, Thor A D. Downregulation of erbB3 abrogates erbB2-mediated tamoxifen resistance in breast cancer cells. Int J Cancer. 2007;120(9):1874-82.

17. Hsieh A C, Moasser M M. Targeting HER proteins in cancer therapy and the role of the non-target HER3. Br J Cancer. 2007;97(4):453-7.

18. Folgiero V, Avetrani P, Bon G, Di Carlo S E, Fabi A, Nistico C, et al. Induction of ErbB-3 expression by alpha6beta4 integrin contributes to tamoxifen resistance in ERbeta1-negative breast carcinomas. PLoS One. 2008;3(2):e1592.

19. Frogne T, Jepsen J S, Larsen S S, Fog C K, Brockdorff B L, Lykkesfeldt A E. Antiestrogen-resistant human breast cancer cells require activated protein kinase B/Akt for growth. Endocr Relat Cancer. 2005;12(3):599-614.

20. Gala K, Chandarlapaty S. Molecular pathways: HER3 targeted therapy. Clin Cancer Res. 2014;20(6):1410-6.

21. Sakai K, Yokote H, Murakami-Murofushi K, Tamura T, Saijo N, Nishio K. Pertuzumab, a novel HER dimerization inhibitor, inhibits the growth of human lung cancer cells mediated by the HER3 signaling pathway. Cancer Sci. 2007;98(9):1498-503.

22. Cai Z, Zhang G, Zhou Z, Bembas K, Drebin J A, Greene M I, et al. Differential binding patterns of monoclonal antibody 2C4 to the ErbB3-p185her2/neu and the EGFR-p185her2/neu complexes. Oncogene. 2008;27(27):3870-4.

23. Junttila T T, Akita R W, Parsons K, Fields C, Lewis Phillips G D, Friedman L S, et al. Ligand-independent HER2/HER3/PI3K complex is disrupted by trastuzumab and is effectively inhibited by the PI3K inhibitor GDC-0941. Cancer Cell. 2009;15(5):429-40.

24. Ren X R, Wei J, Lei G, Wang J, Lu J, Xia W, et al. Polyclonal HER2-specific antibodies induced by vaccination mediate receptor internalization and degradation in tumor cells. Breast cancer research : BCR. 2012;14(3):R89.

25. Stamper C C, Zhang Y, Tobin J F, Erbe D V, Ikemizu S, Davis S J, et al. Crystal structure of the B7-1/CTLA-4 complex that inhibits human immune responses. Nature. 2001;410(6828):608-11.

26. Topalian S L, Drake C G, Pardoll D M. Immune checkpoint blockade: a common denominator approach to cancer therapy. Cancer Cell. 2015;27(4):450-61.

27. Shin D S, Ribas A. The evolution of checkpoint blockade as a cancer therapy: what's here, what's next? Curr Opin Immunol. 2015;33:23-35.

28. Tumeh P C, Harview C L, Yearley J H, Shintaku I P, Taylor E J, Robert L, et al. PD-1 blockade induces responses by inhibiting adaptive immune resistance. Nature. 2014;515(7528):568-71.

29. Osada T, Yang X Y, Hartman Z C, Glass O, Hodges B L, Niedzwiecki D, et al. Optimization of vaccine responses with an E1, E2b and E3-deleted Ad5 vector circumvents pre-existing anti-vector immunity. Cancer Gene Ther. 2009;16(9):673-82.

30. Fourcade J, Sun Z, Pagliano O, Chauvin J M, Sander C, Janjic B, et al. PD-1 and Tim-3 regulate the expansion of tumor antigen-specific CD8(+ ) T cells induced by melanoma vaccines. Cancer Res. 2014;74(4):1045-55.

31. Wong R M, Scotland R R, Lau R L, Wang C, Korman A J, Kast W M, et al. Programmed death-1 blockade enhances expansion and functional capacity of human melanoma antigen-specific CTLs. International immunology. 2007;19(10):1223-34.

32. Karyampudi L, Lamichhane P, Scheid A D, Kalli K R, Shreeder B, Krempski J W, et al. Accumulation of memory precursor CD8 T cells in regressing tumors following combination therapy with vaccine and anti-PD-1 antibody. Cancer Res. 2014;74(11):2974-85.

33. Soares K C, Rucki A A, Wu A A, Olino K, Xiao Q, Chai Y, et al. PD-1/PD-L1 blockade together with vaccine therapy facilitates effector T-cell infiltration into pancreatic tumors. J Immunother. 2015;38(1):1-11.

34. Binder D C, Engels B, Arina A, Yu P, Slauch J M, Fu Y X, et al. Antigen-specific bacterial vaccine combined with anti-PD-L1 rescues dysfunctional endogenous T cells to reject long-established cancer. Cancer immunology research. 2013;1(2):123-33.

35. van Elsas A, Hurwitz A A, Allison J P. Combination immunotherapy of B16 melanoma using anti-cytotoxic T lymphocyte-associated antigen 4 (CTLA-4) and granulocyte/macrophage colony-stimulating factor (GM-CSF)-producing vaccines induces rejection of subcutaneous and metastatic tumors accompanied by autoimmune depigmentation. J Exp Med. 1999;190(3):355-66.

36. Li B, VanRoey M, Wang C, Chen T H, Korman A, Jooss K. Anti-programmed death-1 synergizes with granulocyte macrophage colony-stimulating factor--secreting tumor cell immunotherapy providing therapeutic benefit to mice with established tumors. Clin Cancer Res. 2009;15(5):1623-34.

37. Xia W, Petricoin E F, 3rd, Zhao S, Liu L, Osada T, Cheng Q, et al. An heregulin-EGFR-HER3 autocrine signaling axis can mediate acquired lapatinib resistance in HER2+ breast cancer models. Breast cancer research : BCR. 2013;15(5):R85.

38. Nanda R, Chow L Q, Dees E C, Berger R, Gupta S, Geva R, et al. Pembrolizumab in Patients With Advanced Triple-Negative Breast Cancer: Phase Ib KEYNOTE-012 Study. J Clin Oncol. 2016;34(21):2460-7.

39. Emens L A, Braiteh FS, Cassier P, DeLord J-P, Eder J P, Shen X, et al. Abstract PD1-6: Inhibition of PD-L1 by MPDL3280A leads to clinical activity in patients with metastatic triple-negative breast cancer. Cancer Res. 2015;75(9 Supplement):PD1-6-PD1-6.

40. Bae S Y, La Choi Y, Kim S, Kim M, Kim J, Jung S P, et al. HER3 status by immunohistochemistry is correlated with poor prognosis in hormone receptor-negative breast cancer patients. Breast Cancer Res Treat. 2013;139(3):741-50.

41. He T C, Zhou S, da Costa L T, Yu J, Kinzler K W, Vogelstein B. A simplified system for generating recombinant adenoviruses. Proc Natl Acad Sci U S A. 1998;95(5):2509-14.

42. Hodges B L, Serra D, Hu H, Begy C A, Chamberlain J S, Amalfitano A. Multiply deleted [E1, polymerase-, and pTP-] adenovirus vector persists despite deletion of the preterminal protein. The journal of gene medicine. 2000;2(4):250-9.

43. Amalfitano A, Hauser M A, Hu H, Serra D, Begy C R, Chamberlain J S. Production and characterization of improved adenovirus vectors with the E1, E2b, and E3 genes deleted. J Virol. 1998;72(2):926-33.

44. Rigby R A, Stasinopoulos D M. Generalized additive models for location, scale and shape. Journal of the Royal Statistical Society: Series C (Applied Statistics). 2005;54(3):507-54.

45. Eilers PHC, Marx B D. Flexible smoothing with B-splines and penalties. 1996:89-121.

46. Hastie T, Tibshirani R. Varying-Coefficient Models. Journal of the Royal Statistical Society Series B (Methodological). 1993;55(4):757-96.

47. Holm S. A Simple Sequentially Rejective Multiple Test Procedure. Scandinavian Journal of Statistics. 1979;6(2):65-70.

48. van Buuren S, Fredriks M. Worm plot: a simple diagnostic device for modelling growth reference curves. Statistics in medicine. 2001;20(8):1259-77.

49. DiCiccio T J, Efron B. Bootstrap confidence intervals. 1996:189-228.

50. Team RDC. R: A Language and Environment for Statistical Computing. the R Foundation for Statistical Computing. 2001.

We claim:

1. A vaccine comprising a polynucleotide encoding a HER3 polypeptide.

2. The vaccine of claim 1, wherein the vaccine is capable of eliciting an immune response to a HER3 polypeptide when administered to a subject.

3. The vaccine of claim 1, wherein the HER3 polypeptide comprises SEQ ID NO: 1 or SEQ ID NO: 2 or portions thereof.

4. The vaccine of claim 3, wherein the portion of the HER3 polypeptide comprises the extracellular domain of HER3.

5. The vaccine of claim 3, wherein the portions of HER3 are selected from at least one of SEQ ID NOs: 5-22 or SEQ ID NOs: 27-34.

6. The vaccine of claim 1, further comprising a vaccine vector.

7. The vaccine of claim 6, wherein the vaccine vector comprises the HER3 polypeptide.

8. The vaccine of claim 6, wherein the vaccine vector is selected from the group consisting of an adenovirus vector, adeno-associated virus (AAV), a fowlpox vector, a vaccinia vector, a VEE vector, a herpesviral vector, a polynucleotide vector, and a minicircle vector.

9. A method of treating a cancer or precancer comprising administering the vaccine of claim 1 to a subject having the cancer or precancer, wherein administration of the vaccine to the subject treats the cancer or precancer, reduces the likelihood of the cancer or precancer developing resistance to the cancer therapeutic or reverses resistance of the cancer or precancer to the cancer therapeutic.

10. The method of claim 9, wherein the cancer is HER2 positive.

11. The method of claim 9, wherein the vaccine is administered concurrently with, before or after administration of the cancer therapeutic.

12. The method of claim 11, wherein the cancer therapeutic is an agent targeting HER2, HER1, estrogen receptor, EGFR, or IGF1R.

13. The method of claim 11, wherein the cancer therapeutic is a checkpoint inhibitor.

14. The method of claim 13, wherein the checkpoint inhibitor is selected from the group consisting of an anti-PD-1 agent, an anti-PDL1 agent, and an anti-CTLA-4 agent.

15. The method of claim 9, wherein the cancer or precancer is selected from a breast, prostate, lung, ovarian, colon, rectal, pancreas, bladder, gastric, melanoma, head and neck or liver cancer or precancer.

16. The method of claim 9, wherein the subject develops an immune response to HER3.

17. The method of claim 9, wherein administration of the vaccine results in a reduction of HER3 expression on cancer or precancer cells after administration of the vaccine as compared to the level of HER3 on the cells prior to vaccination.

18. The method of claims 9, wherein administration results in decreased tumor growth rate or decreased tumor size after administration as compared to prior to administration.

19. The method of claim 9, wherein the cancer therapeutic or prevention agent is selected from trastuzumab, lapatinib, cetuximab, pertuzumab and erlotanib.

20. The method of claim 13, wherein the cancer does not develop resistance to the cancer therapeutic.
